(19) United States
(12) Patent Application Publication
(10) Pub. No.: US 2024/0269256 A1
(43) Pub. Date: Aug. 15, 2024

(54) COMPOSITIONS AND METHODS FOR ENHANCED ANTIGEN BINDING PROTEINS

(71) Applicant: HDT Bio Corp., Seattle, WA (US)

(72) Inventors: Jesse Hong-Sae Erasmus, Port Orchard, WA (US); Jacob Freeman Archer, Seattle, WA (US)

(21) Appl. No.: 18/612,850

(22) Filed: Mar. 21, 2024

Related U.S. Application Data
(63) Continuation of application No. PCT/US2022/076787, filed on Sep. 21, 2022.
(60) Provisional application No. 63/246,978, filed on Sep. 22, 2021.

(51) Int. Cl. A61K 39/125; A61K 39/00; C07K 14/005; C12N 9/12; C12N 15/88

(52) U.S. Cl. CPC A61K 39/125; C07K 14/005; C12N 9/127; C12N 15/88; C12Y 207/07048; A61K 2039/53; A61K 2039/55555; C12N 2770/32322

(57) ABSTRACT

The disclosure provides nanoparticle and compound compositions and methods of making and using the same to a nucleic acid encoding a protein, antibody, or functional fragment thereof for administration to a subject. Various nanoparticle carriers are described. In some instances, the nanoparticle component may include a hydrophobic core having an inorganic particle, and optionally a membrane having a cationic lipid.

### CROSS REFERENCE

This application is a continuation International Application No. PCT/US2022/076787, filed Sep. 21, 2022, which claims the benefit of priority to U.S. Provisional Patent Application No. 63/246,978, filed Sep. 22, 2021, the contents of which is incorporated herein by reference in its entirety.

### STATEMENT AS TO FEDERALLY SPONSORED RESEARCH

This invention was made with government support under Contract number 75N93020C00028 awarded by the National Institute of Allergy and Infectious Diseases National Institutes of Health, DHHS. The US government has certain rights in the invention.

### SEQUENCE LISTING

The instant application contains a Sequence Listing which has been submitted electronically in xml format and is hereby incorporated by reference in its entirety. Said xml copy, created on Mar. 18, 2024, is named 201953-717301—SL.xml and is 96,120 bytes in size.

### BACKGROUND

A challenge with RNA-encoded proteins and antibody therapeutics is achieving efficacious levels of the protein or antibody in vivo. Additionally, the discovery and development of antibodies that 1) are suitable for expression from RNA, and 2) potently and specifically neutralize their target, is hampered by a dependence on the isolation of antibody-producing cells from hosts that have either undergone natural exposure to the target pathogen or toxin or have been deliberately immunized with a representative protein. These two requirements present a bottleneck in discovery and development of RNA-encoded antibody therapeutics. Furthermore, in vivo production of virus-like particles (VLPs) derived from non-enveloped viruses has yet to be demonstrated and is complicated by the involvement of nonstructural viral proteases required for processing of the structural polyprotein in trans. Therefore, there is a great unmet need for enhanced nucleic acid-encoded protein and antibody therapeutics that yield a therapeutically meaningful level of protein expression as well as methods for improving the discovery of relevant antibodies, with the intended function, and suitable for expression from nucleic acids, such as RNA.

### BRIEF SUMMARY

Provided herein are compositions, wherein the compositions comprise: a nucleic acid sequence encoding for: a non-enveloped virus binding protein, wherein the non-enveloped virus binding protein comprises a heavy chain variable (V_{H}) region, wherein the non-enveloped virus binding protein specifically binds a structural protein of a non-enveloped virus; and an RNA-dependent RNA polymerase.

Provided herein are compositions, wherein the compositions comprise: an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of the sequences listed in Table 1 (SEQ ID NOS: 1-4) or Table 2 (SEQ ID NOS: 5-7).

Provided herein are compositions, wherein the compositions comprise: a nucleic acid encoding for an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of the sequences listed in Table 1 (SEQ ID NOS: 1-4) or Table 2 (SEQ ID NOS: 5-7).

Provided herein are compositions, wherein the compositions comprise: an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of SEQ ID NOS: 1-7.

Provided herein are compositions, wherein the compositions comprise: a nucleic acid encoding for an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of SEQ ID NOS: 1-7.

Provided herein are compositions, wherein the compositions comprise: a nanoparticle carrier; and a nucleic acid, wherein the nucleic acid comprises: a region encoding for an RNA-dependent RNA polymerase; a region encoding for a non-enveloped virus structural protein; and a region encoding for a virus protease, wherein the virus structural protein is a substrate for the virus protease.

Provided herein are compositions, wherein the compositions comprise: a nanoparticle; and a nucleic acid, wherein the nucleic acid comprises: a region encoding for an RNA polymerase; a region encoding for a virus structural protein, wherein the virus is a non-enveloped virus; and a region encoding for a virus protease, wherein the virus structural protein is a substrate for the virus protease.

Provided herein are suspensions, wherein the suspensions comprise a composition provided herein.

Provided herein are pharmaceutical compositions, wherein the pharmaceutical compositions comprise a composition provided herein; and a pharmaceutical excipient.

Provided herein are methods for treatment of an infection in a subject, the method comprising: administering to a subject, the composition provided herein, the suspension provided herein, or the pharmaceutical composition provided herein, thereby treating the infection in the subject. Further provided herein are methods, wherein the infection is an enterovirus infection, a coxsackievirus infection, a rhinovirus infection, a poliovirus infection, an echovirus infection, or a parechovirus infection.

Provided herein are methods for modulating an immune response in subject, the methods comprising: administering to a subject, the composition provided herein, the suspension provided herein, or the pharmaceutical composition provided herein, thereby modulating an immune in the subject.

Provided herein are methods for treatment of enterovirus infection, the methods comprising: administering to a subject the enterovirus D68 (EV-D68) binding protein as described herein.

Provided herein are methods for treatment of enterovirus infection, the methods comprising: administering to a subject: the nucleic acid as described herein.

Provided herein are methods for antibody generation, the methods comprising: administering to a mammal a composition, wherein the composition supports formation of a non-enveloped viral protein in the mammal and comprises: a carrier; and a nucleic acid, wherein the nucleic acid comprises: a region encoding for an RNA polymerase; a region encoding for a virus structural protein, wherein the virus is a non-enveloped virus; and a region encoding for a virus protease, wherein the virus structural protein is a substrate for the viral protease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1R show schematic representations of exemplary nanoparticle (NP) carriers. FIG. 1A shows an oil-in-water emulsion. FIG. 1B shows a nanostructured lipid carrier (NLC). FIG. 1C shows a nanoparticle having an inorganic nanoparticle in liquid oil. FIGS. 1D and 1M show a nanoparticle having a cationic lipid membrane, an inorganic nanoparticle, a liquid oil core and a nucleic acid. FIG. 1E shows an oil-in-water emulsion with two or more RNA or DNA molecules. FIG. 1F shows a nanostructured lipid carrier (NLC) with two or more RNA or DNA molecules. FIG. 1G shows a nanoparticle having an inorganic nanoparticle in liquid oil two or more RNA or DNA molecules. FIGS. 1H and 1N show a nanoparticle having a cationic lipid membrane, inorganic particles, a liquid oil core, and two or more RNA or DNA molecules. FIGS. 1I and 1O show a nanoparticle having a cationic lipid membrane, a liquid oil core (e.g., squalene), and a single nucleic acid molecule. FIGS. 1J and 1P show a nanoparticle having a cationic lipid membrane, a liquid oil core (e.g., squalene), and two or more RNA or DNA molecules. FIGS. 1K and 1Q show a nanoparticle having a cationic lipid membrane, a solid core (e.g., glyceryl trimyristate-dynasan), and a single nucleic acid molecule. FIGS. 1L and 1R show a nanoparticle having a cationic lipid membrane, a solid core (e.g., glyceryl trimyristate-dynasan), and two or more RNA or DNA molecules. Drawings not to scale.

FIG. 2 shows the time measurements of nanoparticle size as measured by dynamic light scattering (DLS). X axis is weeks and Y axis is nm diameter. Three time courses correspond to storage at 4, 25, and 42 degrees Celsius.

FIG. 3A shows a maximum-likelihood phylogenetic tree of contemporary EV-D68 isolates from recent outbreaks (since 2010).

FIG. 3B shows variation in protein sequence between 6 isolates of EV-D68.

FIG. 4A shows design of repRNAs encoding EV-D68 P1 followed by either an internal ribosomal entry site (IRES) or thosea asigna virus 2A (T2A) ribosomal skipping peptide, and then the 3CD protein.

FIG. 4B shows anti-VP1 Western blots of BHK cells transfected with either 25 or 250 ng of repRNA in triplicate transfections.

FIG. 4C shows densitometry analyses of Western blots.

FIG. 4D shows 80% plaque reduction neutralization test (PRNT₈₀) titers in C57BL/6 mice 14 days after the prime and boost vaccinations with 10 μg of each repRNA formulated with NP-1.

FIG. 5A shows design of repRNAs encoding two EV-D68 proteins from 6 different strains of EV-D68, each encoding the P1 followed by IRES and then the 3CD protein. Additionally, a repRNA with the 3CD protein open reading frame deleted to test the importance of 3CD in the generation of VLPs.

FIG. 5B shows anti-VP1 Western blots of BHK cells transfected with 250 ng of each repRNA described in FIG. 5A.

FIG. 5C shows 80% plaque reduction neutralization test titers in alpacas before immunization and 14 days after the prime as well as 14 days after boost immunization comprised of the 6 repRNAs described in FIGS. 5A and 5B in a multivalent vaccination formulated with NP-1.

FIG. 6A shows cell index values in each well of a 96-well ePlate seeded with rhabdomyosarcoma cells then infected with EV-D68 complexed with a unique antibody per well in order to screen a library of antibodies for EV-D68 neutralizing activity.

FIG. 6B shows the phylogenetic relationship between a selection of antibodies from FIG. 6A followed by quantitative data for each collected in FIG. 6A, including the neutralizing area under the curve (nAb AUC), binding antibody optical density (bAb OD), and the ratio of nAb to bAb. Additionally, the CDR3 amino acid sequences are shown for each antibody identified. Figure discloses SEQ ID NOS 7, 7, 7, 7, 7, 7, 7, 7, 7, 22, 22, 22, 22, 22, 22, 22-25 and 25, respectively, in order of appearance.

FIG. 6C shows the 50% inhibitory concentrations (IC₅₀) of 4 selected antibodies, identified in FIGS. 6A and 6B, and expressed and purified in E. coli.

FIGS. 7A-7F show SEAP levels in BALB/c mice injected intramuscularly with various embodiments of lipid nanoparticle formulations described herein. FIG. 7A shows SEAP levels on day 4 post-injection. FIG. 7B shows SEAP levels on day 6 post-injection. FIG. 7C shows SEAP levels on day 8 post-injection. FIG. 7D shows SEAP levels on day 4 post-injection. FIG. 7E shows SEAP levels on day 6 post-injection. FIG. 7F shows SEAP levels on day 8 post-injection. X-axis: Condition, Y-axis: Relative light units (RLU).

FIG. 8 is a bar chart with measurements of Z-average measurement and polydispersity index (PDI) on the Y-axis and group number on the X-axis for conditions 1 to 14.

FIGS. 9A-9B show dot charts showing anti-D614G IgG levels for conditions 1 to 14. FIG. 9A shows a dot chart with IgG (μg/ml) on the Y-axis, group number on the X-axis for conditions 1 to 14. Measurements were recorded at day 14 for anti-D614G (1:40 dilution) IgG responses. FIG. 9B shows a dot chart with IgG (μg/ml) on the Y-axis, group number on the X-axis for conditions 1 to 14. Measurements were recorded at day 28 for anti-D614G (1:200 dilution) IgG responses.

FIG. 10 shows a dot chart at day 28 with anti-D614G (1:200 dilution) IgG (ug/ml) measurements on the Y-axis and indications of storage conditions on the X-axis.

FIGS. 11A-11B show graphs of the percent neutralization of various clades of enterovirus by the G12 monomer and the G12-Fc dimer construct. FIG. 11A shows percent neutralization for the recombinant V_{H}H G12. FIG. 11B shows percent neutralization for V_{H}H G12 fused with the Fc domain of human IgG1. X-axis: nM, Y-axis: percent (%) neutralization.

FIGS. 12A-12B show various EV-D68 repRNA constructs and their ability to induce neutralizing antibody responses in C57BL/6 mice. FIG. 12A shows a schematic of (1) P1_{IRES}-3CD: full-length P1-IRES-3CD protease repRNA construct; (2) P1_{Δ3D}: a P1-IRES construct without the 3CD protease; (3) P1_{T2A}: a P1 construct with T2A separating the VP subunits in the P1 polyprotein; and (4) VP1_{HA2}: a VP1 construct fused to influenza HA2. FIG. 12B shows a graph of the 50% neutralization titer in serum collected from C57BL/6 mice receiving prime/boost of each construct in FIG. 12A.

FIG. 13 shows a schematic of non-human primate immunization and blood draws schedule.

FIG. 14 shows graphs of the percent neutralization for EV-D68 repRNA and CCHFV rep RNA controls.

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

### DETAILED DESCRIPTION OF THE INVENTION

Provided herein are compositions, kits, methods, and uses thereof for treatment of various conditions. Briefly, further described herein are (1) nucleic acids coding for proteins, antibodies, and RNA polymerases; (2) nanoparticle carriers systems; (3) combination compositions; (4) thermally stable, dried, and lyophilized vaccines; (5) pharmaceutical compositions; (6) dosing; (7) administration; (8) therapeutic applications; and (9) kits.

Compositions provided herein provide several advantages over preceding therapeutic formulations such as a protective nanoparticle configuration for safe and efficient nucleic acid delivery, a self-replicating RNA polymerase for the transcription of the nucleic acid. Provided herein are methods for 1) driving potent neutralizing antibody responses against conformationally-native epitopes on virus like particles (VLPs) expressed from RNA, and the discovery and isolation of antibodies raised against those immunogens. Further provided herein, are compositions that co-express the P1 and 3CD proteins of enteroviruses in vivo, which results in efficient formation of VLPs and robust neutralizing antibody responses that can be mined for the development of anti-viral therapeutics.

### Definitions

Throughout this disclosure, various embodiments can be presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

As used herein, “optional” or “optionally” means that the subsequently described circumstance may or may not occur, so that the description includes instances where the circumstance occurs and instances where it does not.

Unless specifically stated or apparent from context, as used herein, the term “about” in reference to a number or range of numbers is understood to mean the stated number and numbers +/−20% thereof, or 20% below the lower listed limit and 20% above the higher listed limit for the values listed for a range.

The term “effective amount” or “therapeutically effective amount” refers to an amount that is sufficient to achieve or at least partially achieve the desired effect.

### Nucleic Acid

Provided herein are compositions comprising a nucleic acid or a plurality of nucleic acids. Provided herein are compositions comprising a nucleic acid encoding for a protein, an antibody, or a functional fragment thereof. In some embodiments, the nucleic acid is in complex with a nanoparticle. In some embodiments, the nucleic acid is in complex with a membrane of the nanoparticle. In some embodiments, the nucleic acid is in complex with a hydrophilic surface of the nanoparticle. In some embodiments, the nucleic acid is within the nanoparticle. In some embodiments, the nucleic acid is within a hydrophobic core.

In some embodiments, nucleic acids provided herein comprise a deoxyribonucleic acid (DNA), a ribonucleic acid (RNA), a peptide nucleic acid (PNA), or a combination thereof. In some embodiments, compositions provided herein comprise one or more types of nucleic acid sequences. In some embodiments, compositions provided herein comprise two or more types of nucleic acid sequences. In some embodiments, compositions provided herein comprise at least one DNA molecule. In some embodiments, compositions provided herein comprise at least one RNA molecule. In some embodiments, compositions provided herein comprise at least one DNA molecule and at least one RNA molecule. The nucleic acid may be linear or include a secondary structure (e.g., a hair pin). In some embodiments, the nucleic acid is a polynucleotide comprising modified nucleotides or bases, and/or their analogs. A polynucleotide may comprise modified nucleotides, such as methylated nucleotides and their analogs. If present, modification to the nucleotide structure may be imparted before or after assembly of compositions provided herein. Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A (N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); m1I (1-methylinosine); m′Im (1,2′-O-dimethylinosine); m3C (3-methylcytidine); Cm (2T-O-methylcytidine); s2C (2-thiocytidine); ac4C (N4-acetylcytidine); f5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); hoSU (5-hydroxyuridine); moSU (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethylaminomethyl-2-L-Omethyluridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-0-dimethyladenosine); m62Am (N6,N6,O-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); f5Cm (5-formyl-2′-O-methylcytidine); m1Gm (1,2′-O-dimethylguanosine); m′Am (1,2-O-dimethyl adenosine) irinomethyluridine); tm5s2U (S-taurinomethyl-2-thiouridine)); imG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Any one or any combination of these modified nucleobases may be included in the self-replicating RNA of the invention. Many of these modified nucleobases and their corresponding ribonucleosides are available from commercial suppliers. If desired, the nucleic acid can contain phosphoramidate, phosphorothioate, and/or methylphosphonate linkages. The RNA sequence can be modified with respect to its codon usage, for example, to increase translation efficacy and half-life of the RNA. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methyltransferase, which catalyzes the construction of N7-monomethylated cap 0 structures). Cap structure can provide stability and translational efficacy to the RNA molecule. The 5′ cap of the RNA molecule may be further modified by a 2′-O-Methyltransferase which results in the generation of a cap 1 structure (m7Gppp [m2′-O] N), which may further increase translation efficacy. A cap 1 structure may also increase in vivo potency. If present, modification to the nucleotide structure may be imparted before or after assembly of compositions provided herein.

In some embodiments, nucleic acids provided herein are present in an amount of above 5 ng to about 1 mg. In some embodiments, nucleic acids provided herein are present in an amount of up to about 25, 50, 75, 100, 150, 175 ng. In some embodiments, nucleic acids provided herein are present in an amount of up to about 1 mg. In some embodiments, nucleic acids provided herein are present in an amount of about 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, nucleic acids provided herein are present in an amount of 0.05 μg, 0.1 μg, 0.2 μg, 0.5, μg 1 μg, 5 μg, 10 μg, 12.5 μg, 15 μg, 25 μg, 40 μg, 50 μg, 100 μg, 200 μg, 300 μg, 400 μg, 500 μg, 600 μg, 700 μg, 800 μg, 900 μg, 1 mg. In some embodiments, the nucleic acid is at least about 200, 250, 500, 750, 1000, 2000, 3000, 4000, 5000, 6000, 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is up to about 7000, 8000, 9000, 10,000, 11,000, 12,000, 13,000, 14,000, 15,000, 16,000, 17,000, 18,000, 19,000, or 20,000 nucleotides in length. In some embodiments, the nucleic acid is about 7500, 10,000, 15,000, or 20,000 nucleotides in length.

### RNA Encoding for Proteins

Provided here are compositions comprising a nucleic acid encoding for a protein or a functional fragment thereof. In some embodiments, the protein is an antigen, an antigen-binding protein, or a fragment thereof. In some embodiments, the antigen is an antigen from a microbial organism. In some embodiments, the antigen is a microbial antigen. In some embodiments, the antigen is a bacterial antigen. In some embodiments, the microbial antigen is a viral antigen. In some embodiments, the viral antigen is a surface protein or a transmembrane protein. In some embodiments, the viral antigen is a spike protein, a glycoprotein, or an envelope protein. In some embodiments, the viral antigen is expressed cytosolically by a host cell or is not secreted by a host cell. In some embodiments, more than one antigen is encoded by a single nucleic acid. In some embodiments, the viral antigen is derived from a non-enveloped virus. In some embodiments, the viral antigen is derived from an enveloped virus. In some embodiments, more than one antigen is derived from a non-enveloped virus. In some embodiments, more than one antigen is derived from an enveloped virus.

Enveloped viruses fuse the viral envelope with a host cellular membrane. Fusion of some enveloped viruses occurs within the low-pH environment of an acidic endosomal compartment. Enveloped viruses typically reach the endosomal compartment via trafficking in clathrin-coated vesicles or the caveolar route. Examples of enveloped viruses include but are not limited to coronaviruses, influenza A, hepatitis C virus, and human immunodeficiency virus (HIV).

Non-enveloped viruses do not use the host secretory system to enter or leave a host cell during infection. Instead, non-enveloped viruses enter the cytosol by directly penetrating the plasma membrane, as well as through a variety of endocytic mechanisms leading to penetration of internal membrane(s), the Golgi, and the endoplasmic reticulum of the host cell. Enteroviruses enter the host cell by receptor-mediate endocytosis. Following endocytosis, uncoating of the virion occurs in the endosome and the positive-stranded RNA along with the covalently-linked VPg protein is released into the cytoplasm. Viral RNA is translated by host ribosomes making a single polyprotein that is catalytically cleaved by enterovirus proteases 2Apro and 3Cpro. After production and accumulation of non-structural proteins, including the viral polymerase, viral RNA is then replicated using the virally-encoded RNA-dependent RNA polymerase to generate a double-stranded RNA. The negative sense RNA serves as the template to make more positive sense RNA. Newly produced RNA can be the template to produce more positive sense RNAs or serve as the genome for progeny viruses. Capsid proteins assemble and newly synthesized positive-stranded viral RNA is packaged into virion. Finally, new progeny virions are released either by non-lytic release, where virions are released in vesicles, or are released when the cell undergoes lysis (lytic release).

Provided herein are compositions comprising a nucleic acid encoding for a viral antigen derived from a non-enveloped virus. In some embodiments, the non-enveloped virus is a double-stranded DNA virus. In some embodiments, the non-enveloped virus is a single-stranded DNA virus. In some embodiments, the non-enveloped virus is a double-stranded RNA virus. In some embodiments, the non-enveloped virus is a single-stranded RNA virus.

In some embodiments, the non-enveloped virus is selected from the virus families listed below:

double-stranded DNA
viruses
Adenoviridae
Iridoviridae
Papillomaviridae
Polyomaviridae
single-stranded DNA
viruses
Anellovirus
Circoviridae
Parvoviridae
double-stranded RNA
viruses
Birnaviridae
Picobirnaviridae
Reoviridae
single-stranded RNA
viruses
Picornaviridae
Astroviridae
Caliciviridae
Hepevirus
Nodaviridae

In some embodiments, the viral antigen is derived from a Picornaviridae. In some embodiments, the viral antigen is derived from an enterovirus, a coxsackievirus, a rhinovirus, a poliovirus, an echovirus, or a parechovirus. In some embodiments, the viral antigen is derived from an enterovirus. Enteroviruses have a single open reading frame divided into the P1 and nonstructural P2-P3 polyproteins. P1 is divided into capsid proteins VP1, VP2, VP3, and VP4. P3 contains a 3CD protease which cleaves P1 into the four capsid monomers. In some embodiments, the enterovirus is an enterovirus D68 (EV-D68), an enterovirus A71 (EV-A71), a coxsackievirus A6 (CV-A6), or a coxsackievirus B3 (CV-B3). In some embodiments, the enterovirus is enterovirus D68 (EV-D68). In some embodiments, the EV-D68 belongs to clade A. In some embodiments, the EV-D68 belongs to clade B. In some embodiments, the EV-D68 belongs to clade C. In some embodiments, the EV-D68 belongs to clade D. In some embodiments, the EV-D68 is US/MO/14-18947-EV-D68. In some embodiments, compositions provided herein comprise a nucleic acid encoding for a protein, an antibody, or an antibody fragment that binds to an antigen from a Picornaviridae. In some embodiments, compositions provided herein comprise a nucleic acid encoding for a protein, an antibody, or an antibody fragment that binds to an enterovirus or an enterovirus antigen. In some embodiments, compositions provided herein comprise a nucleic acid encoding for a protein, an antibody, or an antibody fragment that binds to a VP1 capsid protein. In some embodiments, compositions provided herein comprise a nucleic acid encoding for a protein, an antibody, or an antibody fragment that binds to a VP2 capsid protein. In some embodiments, compositions provided herein comprise a nucleic acid encoding for a protein, an antibody, or an antibody fragment that binds to a VP3 capsid protein. In some embodiments, compositions provided herein comprise a nucleic acid encoding for a protein, an antibody, or an antibody fragment that binds to a VP4 capsid protein.

Further provided herein are nucleic acids encoding for a structural protein from a non-enveloped virus and a 3CD protease. In some embodiments, the nucleic acids encoding for a structural protein from a non-enveloped virus and a 3CD protease further comprise an IRES sequence. In some embodiments, the nucleic acids encoding for a structural protein from a non-enveloped virus and a 3CD protease further comprise a non-structural protein from an alphavirus.

### RNA Encoding for Antigen Binding Molecules

Provided here are compositions comprising a nucleic acid encoding for an antibody or an antibody fragment. In some embodiments, the antibody is a monoclonal antibody. Monoclonal antibodies or mAbs include intact molecules, as well as antibody fragments (such as, Fab and F(ab′)2 fragments) that are capable of specifically binding to an epitope of a protein or antigen. In some embodiments, the composition comprises nucleic acids encoding for polyclonal antibody.

In some embodiments, the antibody is a murine antibody, a humanized antibody, or a fully human antibody, or a single domain heavy chain antibody derived from camelids, sharks, eels, or other species that produce such single-domain antibodies. In some embodiments, the antibody is an immunoglobulin (Ig) molecule. Immunoglobulin (Ig) molecules and immunologically active portions of immunoglobulin molecules (i.e., molecules that contain an antigen binding site that specifically bind an antigen) are comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art. Non-limiting embodiments of which are discussed below, and include but are not limited to a variety of forms, including full length antibodies and antigen-binding portions thereof; including, for example, an immunoglobulin molecule, a monoclonal antibody, a chimeric antibody, a CDR-grafted antibody, a human antibody, a humanized antibody, a single chain antibody, a Fab, a F(ab′), a F(ab′)2, a Fv antibody, fragments produced by a Fab expression library, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies. In some embodiments, the immunoglobulin molecule is an IgG, IgE, IgM, IgD, IgA, or an IgY isotype immunoglobulin molecule. In some embodiments, the antibody or immunoglobulin molecules provided herein are a specific subclass of immunoglobulin molecule. In some embodiments, the immunoglobulin molecule is an IgG1, an IgG2, an IgG3, an IgG4, an IgGA1, or an IgGA2 subclass immunoglobulin molecule. In a full-length antibody, each heavy chain is comprised of a heavy chain variable domain (abbreviated herein as HCVR or V_{H}) and a heavy chain constant region. The heavy chain constant region is comprised of three domains: C_{H}1, C_{H}2, and C_{H}3. Each light chain is comprised of a light chain variable domain (abbreviated herein LCVR as V_{L}) and a light chain constant region. The light chain constant region is comprised of one domain, CL. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. This structure is well-known to those skilled in the art. The chains are usually linked to one another via disulfide bonds. Furthermore, in humans, the light chain may comprise a kappa chain or a lambda chain. Complementarity Determining Regions (“CDRs”), i.e., CDR1, CDR2, and CDR3) are the amino acid residues of a heavy or light chain variable domain specific for antigen binding. Each variable domain typically has three CDR regions identified as CDR1, CDR2 and CDR3. Each complementarity determining region can comprise amino acid residues from a “complementarity determining region” as defined by Kabat (i.e., about residues 24-34 (L1), 50-56 (L2) and 89-97 (L3) in the light chain variable domain and 31-35 (HI), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (LI), 50-52 (L2) and 91-96 (L3) in the light chain variable domain and 26-32 (HI), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop. The exact boundaries of these CDRs have been defined differently according to different systems. The system described by Kabat (Kabat et al, Sequences of Proteins of Immunological Interest (National Institutes of Health, Bethesda, Md. (1987) and (1991)) not only provides an unambiguous residue numbering system applicable to any variable region of an antibody, but also provides the residue boundaries defining the three CDRs. These CDRs may be referred to as Kabat CDRs. Chothia and coworkers (Chothia & Lesk, J. Mol. Biol, 196:901-917 (1987) and Chothia et al., Nature 342:877-883 (1989)) found that certain sub-portions within Kabat CDRs adopt nearly identical peptide backbone conformations, in spite of great diversity at the level of amino acid sequence. These sub-portions were designated as LI, L2 and L3 or HI, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB). 9: 133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or assay result that particular residues or groups of residues or even entire CDRs do not significantly impact antigen binding. The alignment of the CDR sequences can be conducted using publicly available software such as BLAST, Align, and the international ImMunoGeneTics information system (IMGT). Those skilled in the art can determine the appropriate parameters for alignment, but the default parameters for BLAST are specifically contemplated.

In some embodiments, nucleic acids provided herein encode for a recombinant antibody, a chimeric antibody, or a multivalent antibody. In some embodiments, the multivalent antibody is a bispecific antibody, a trispecific antibody, or a multispecific antibody. In some embodiments, the antibody or functional fragment is an antigen-binding fragment (Fab), and Fab2 a F(ab′), a F(ab∝)2, an dAb, an Fc, a Fv, a disulfide linked Fv, a scFv, a tandem scFv, a free LC, a half antibody, a single domain antibody (dAb), a diabody, or a nanobody. In some embodiments, nucleic acids provided herein encode for a single variable domain on a heavy chain (also referred to as a nanobody or a V_{H}H). In some embodiments, the nanobody comprises a heavy chain variable (V_{H}) region. In some embodiments, the nanobody comprises one CDR region. In some embodiments, the nanobody comprises CDR1, CDR2, or CDR3. In further embodiments, the heavy chain variable (V_{H}) region comprises three CDR regions. In some embodiments, the antibody, nanobody, or fragment thereof is modified with the addition of a glycosylphosphatidylinositol (GPI) anchor such as that derived from CD55, or with a human Fc domain, or a combination of Fc and GPI. In some embodiments, nucleic acids provided herein encode for a nanobody that specifically binds to a viral structural protein. In some embodiments, the viral structural protein is derived from a non-enveloped virus. Viral antigen binding molecules are discussed further below. In some embodiments, an antibody, an antibody fragment, or a nanobody provided herein is originally generated by a non-human animal (e.g., sheep, dog, rabbit, mouse, rat, non-human primate, goat, llama, alpaca, camels, and horse) against an antigen described herein and, optionally, humanized as described herein. In some embodiments, an antibody, an antibody fragment, or a nanobody provided herein is originally generated in a camelid animal. In some embodiments, an antibody, an antibody fragment, or a nanobody provided herein is originally generated in an alpaca, a camel, or a llama.

### Viral Antigen Binding Molecules

Provided herein are compositions comprising a nucleic acid encoding for a protein, antibody, antibody fragment, or nanobody that binds to a microbial antigen. In some embodiments, the microbial antigen is derived from a bacterium, a fungus, a parasite, or a virus. In some embodiments, the microbial antigen is a viral protein. In some embodiments, the viral protein is a structural protein. In some embodiments, the viral protein is a non-structural protein. In some embodiments, the structural protein is a capsid protein. In some embodiments, the protein, the antibody, the antibody fragment, or the nanobody binds to a Picornaviridae protein. In some embodiments, the protein, the antibody, the antibody fragment, or the nanobody binds to an enterovirus protein. In some embodiments, the protein, the antibody, the antibody fragment, or the nanobody binds to an enterovirus D68 (EV-D68) protein.

Exemplary amino acid sequences for EV-D68 antibodies and antibody fragments thereof are provided below in Table 1. Nucleic acids described here may encode for, when translated by cellular machinery, a protein. In some embodiments, the nucleic acid encodes for protein having a sequence of any one of SEQ ID NOS: 1 to 4. In some embodiments, the nucleic acid comprises a region encoding for a protein sequence of SEQ ID NO: 1. In some embodiments, the nucleic acid comprises a region encoding for a protein sequence of SEQ ID NO: 2. In some embodiments, the nucleic acid comprises a region encoding for a protein sequence of SEQ ID NO: 3. In some embodiments, the nucleic acid comprises a region encoding for a protein sequence of SEQ ID NO: 4. In some embodiments, the nucleic acid comprises a region encoding for one or more CDR3 loop sequences. In some embodiments, the nucleic acid comprises a region encoding for a CDR3 loop sequence provided in FIG. 63 or Table 2. In some embodiments, the nucleic acid comprises a region encoding for a CDR3 loop sequence selected from AAA_B1, AAA_H1, AAA_F9 or AAA_G12 as listed in FIG. 63. In some embodiments, the nucleic acid region has at least 80%, 85%, 90%, 95%, 99% or more sequence identity to asequence listed in Table 1 or Table 2. Percent identity can be calculated using alignment methods known in the art, for instance alignment of the sequences can be conducted using publicly available software such as BLAST, Align, ClustalW2. Those skilled in the art can determine the appropriate parameters for alignment, but the default parameters for BLAST are specifically contemplated.

TABLE 1
Anti-EV-D68 (V_{H}H) Amino Acid Sequences

| SEQ ID | | |
|---|---|---|
| NO: | VHH | SEQUENCE |
| 1 | B1 | MKYLLPTAAAGLLLLAAQPAMAGPGAAAQVQLAESGGGLAQPGGSLRLSCAASGSIF |
| | | SIDAMGWYRQAIGIQRELVAAITSGGSTNYADSVKGRFTISRGNAKNTVYLQMNSL |
| | | KPEDTAVYYCNADDETNYERYWGQGTQVTVSSAHHSEDPSARQACTSGAPVPYP |
| | | DPLEPRAA* |
| 2 | H1 | MKYLLPTAAAGLLLLAAQPAMAGPGAAAQLQLVETGGLVQAGGSLRLSCTASGRTFS |
| | | SEAMAWFRQAPGKEREFVATINWSSGTDYADSVKGRFTISRDNTKNTVTVYLQMN |
| | | SLKPEDTAVYYCAADRTGWGASGRDSYEYDLWGQGTQVTVSSEPKTPKPQPARQA |
| | | CTSGAPVPYPDPLEPRAA* |
| 3 | F9 | MKYLLPTAAAGLLLLAAQPAMAGPGAAAQLQLVESGGGLVQPGGSLRLSCAASGRVI |
| | | GINAMGWYRQAPGKQRELVARVTQAGNINYADSVKDRFTISRDKAENAVYLQMN |
| | | SLKPEDTAVYYCNGDLFDTPWGPSNDYWGQGTQVTVSSEPKTPKPQPARQACTSG |
| | | APVPYPDPLEPRAA* |
| 4 | G12 | MKYLLPTAAAGLLLLAAQPAMAGPGAAAQVQLVESGGGLVQPGGSLRLSCLASGITFT |
| | | VYRMAWYRQAPGRQRDLVAEVAPGGGTVAANSVKGRFTISRDSAKNTVDLQMND |
| | | LKPDDTAVYYCYARNLFTSGEYWGQGTQVTVSSEPKTPKPQPARQACTSGAPVPY |
| | | PDPLEPRAA* |

Signal peptide: Italicized
VHH sequence: Nonunderlined CAPS
Bold: Etag

TABLE 2
EV-D68 Binding CDR3 Loop Sequences

| SEQ | | |
|---|---|---|
| ID NO: | Reference | CDR3 Loop Sequence |
| 5 | AAA_H1 | CAADRTGWGASGRDSYEDLWGQGTQVTVS |
| 6 | AAA_F9 | CNGDLFDTPWGPSNDYGWGTQVTVS |
| 7 | AAA_G12 | CAADRTGWGASGRDSYEYDLWGQGTQVTVS |

### RNA Encoding for an RNA Polymerase

Provided herein are compositions comprising a self-replicating nucleic acid. In some embodiments, compositions provided herein comprise one or more nucleic acids. In some embodiments, compositions provided herein comprise two or more nucleic acids. In some embodiments, nucleic acids provided herein code for an RNA polymerase. In some embodiments, nucleic acids provided herein code for a viral RNA polymerase. In some embodiments, nucleic acids provided herein code for: (1) a viral RNA polymerase; and (2) a protein, antibody, or functional fragment thereof. In some embodiments, compositions provided herein comprise a first nucleic acid encoding for a viral RNA polymerase; and a second nucleic acid encoding for a protein, antibody, or functional fragment thereof.

Provided herein are compositions comprising a self-replicating RNA. A self-replicating RNA (also called a replicon) includes any genetic element, for example, a plasmid, cosmid, bacmid, phage or virus that is capable of replication largely under its own control. Self-replication provides a system for self-amplification of the nucleic acids provided herein in mammalian cells. In some embodiments, the self-replicating RNA is single stranded. In some embodiments, the self-replicating RNA is double stranded.

An RNA polymerase provided herein can include but is not limited to: an alphavirus RNA polymerase, an Eastern equine encephalitis virus (EEEV) RNA polymerase, a Western equine encephalitis virus (WEEV), Venezuelan equine encephalitis virus (VEEV), Also, Chikungunya virus (CHIKV), Semliki Forest virus (SFV), or Sindbis virus (SINV). In some embodiments, the RNA polymerase is a VEEV RNA polymerase. In some embodiments, the nucleic acid encoding for the RNA polymerase comprises at least 85% identity to the nucleic acid sequence of SEQ ID NO: 8. In some embodiments, the nucleic acid encoding for the RNA polymerase comprises at least 90% identity to the nucleic acid sequence of SEQ ID NO: 8. In some embodiments, the nucleic acid encoding for the RNA polymerase comprises at least 95% identity to the nucleic acid sequence of SEQ ID NO: 8. In some embodiments, the nucleic acid encoding for the RNA polymerase comprises at least 99% identity to the nucleic acid sequence of SEQ ID NO: 8. In some embodiments, the nucleic acid encoding for the RNA polymerase is SEQ ID NO: 8.

In some embodiments, the amino acid sequence for VEEV RNA polymerase comprises at least 85% identity to RELPVLDSAAFNVECFKKYACNNEYWETFKENPIRLTEEN VVNYITKLKGP (SEQ ID NO: 9) or TQMRELPVLDSAAFNVECFKKYACNNEYWE TFKENPIRLTE (SEQ ID NO: 10). In some embodiments, the amino acid sequence for VEEV RNA polymerase comprises at least 90% identity to SEQ ID NO: 9, SEQ ID NO: 10, or SEQ ID NO: 11. In some embodiments, the amino acid sequence for VEEV RNA polymerase comprises at least 95% identity to SEQ ID NO: 9, SEQ ID NO: 10, or SEQ ID NO: 11. In some embodiments, the amino acid sequence for VEEV RNA polymerase comprises at least 99% identity to SEQ ID NO: 9, SEQ ID NO: 10, or SEQ ID NO: 11. In some embodiments, the amino acid sequence for VEEV RNA polymerase is SEQ ID NO: 9, SEQ ID NO: 10, or SEQ ID NO: 11.

Provided herein are compositions and methods comprising replicon RNA (repRNA) encoding for one or more structural proteins from a non-enveloped virus. In some embodiments, the repRNA encodes the EV-D68 P1 polyprotein. In some embodiments, the repRNA encodes a protease. In some embodiments, the repRNA encodes the 3CD protease. In some embodiments, the structural protein and the protease are co-expressed. In further embodiments, the repRNA comprises one or more open reading frames. In some embodiments, the open reading frames are separated by an internal ribosomal entry site (IRES). In some embodiments, the open reading frames are separated by a ribosomal skipping peptide sequence. In some embodiments the ribosomal skipping peptide sequence is from Thosea asigna virus (T2A).

### Nanoparticle Carrier Systems

Provided herein are various compositions comprising a nanoparticle carriers or a plurality of nanoparticle carriers. Nanoparticle carriers are also referred to herein as carriers, nanoparticles, or abbreviated as NPs. Nanoparticles provided herein can be organic, inorganic, or a combination of inorganic and organic materials that are less than about 1 micrometer (pm) in diameter. In some embodiments, nanoparticles provided herein are used as a delivery system for a bioactive agent (e.g., a nucleic acid encoding a protein, an antibody, an antibody fragment, a nanobody, or a functional fragment thereof as provided herein). In some embodiments, the nanoparticle carrier provided herein is a lipid nanoparticle (also referred to as a lipid carrier).

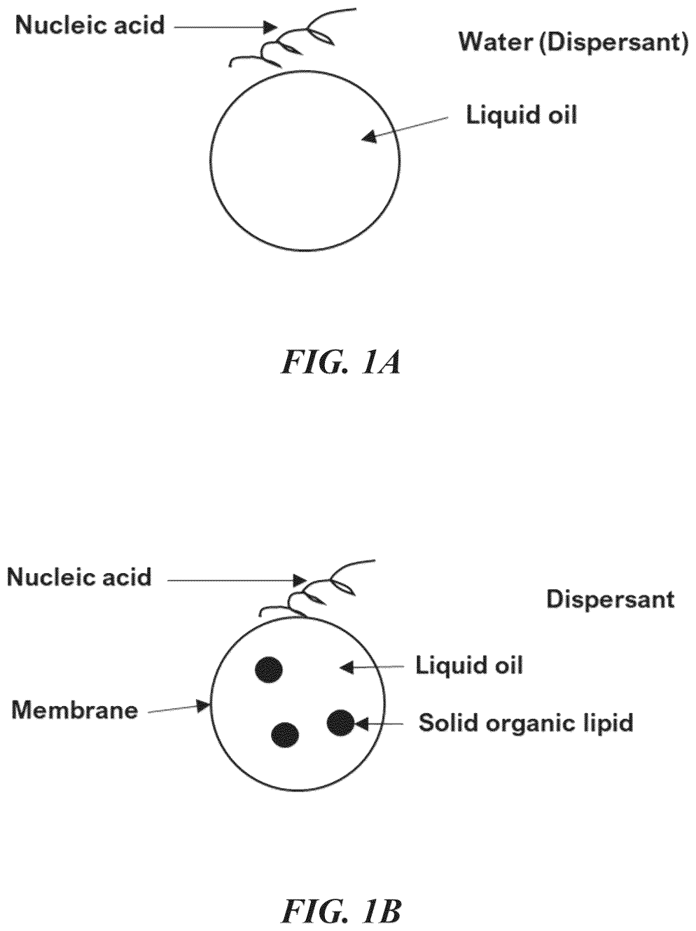

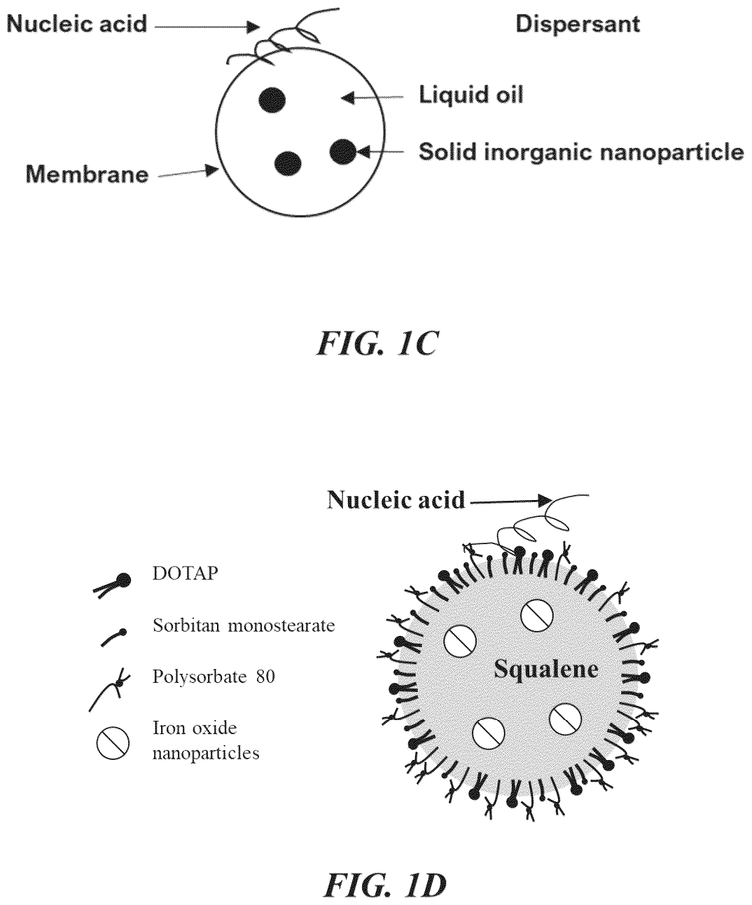

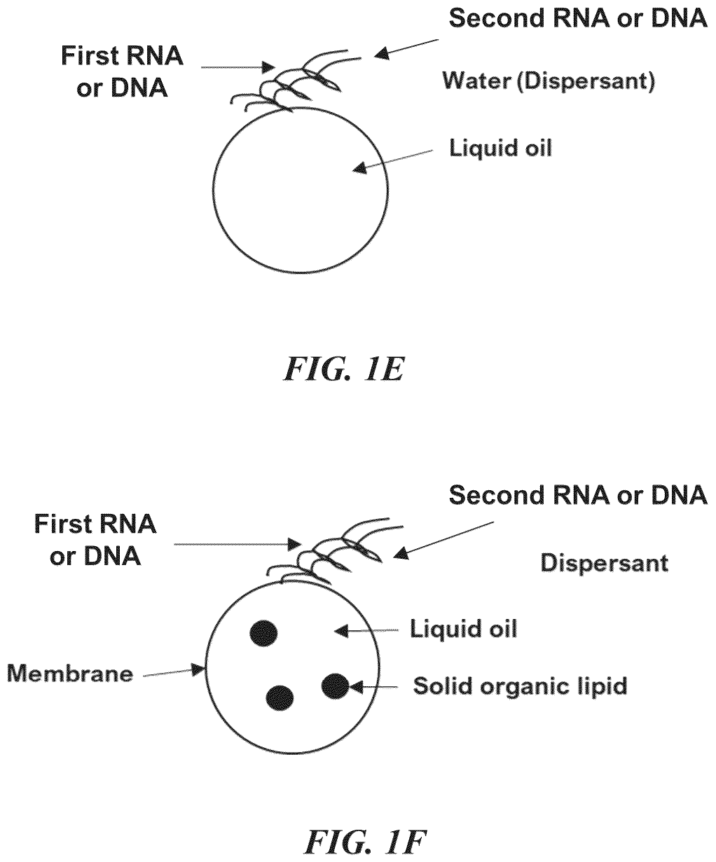

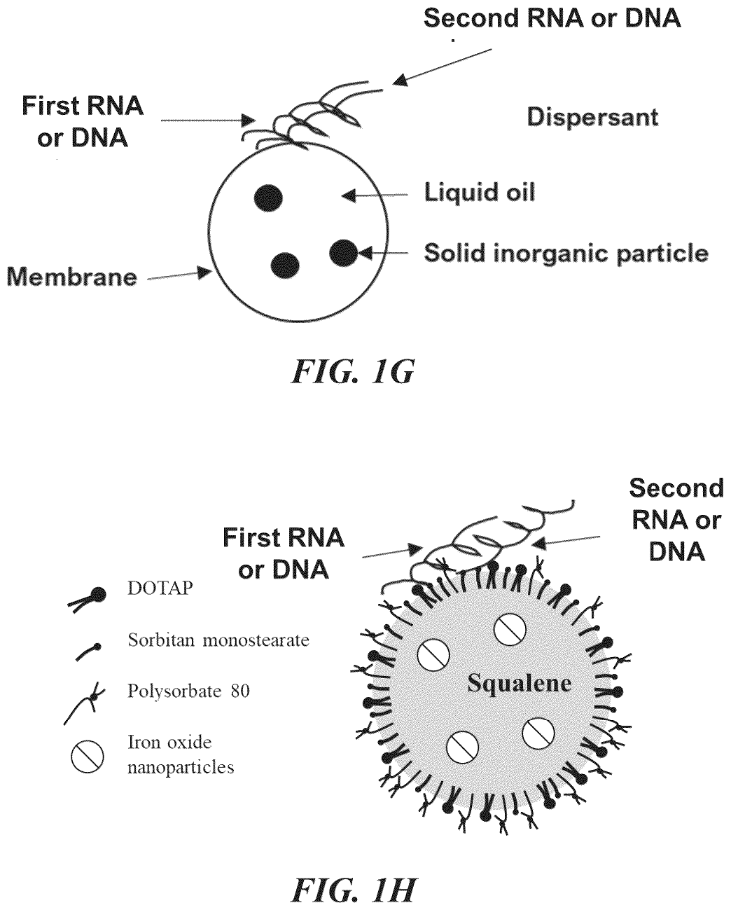

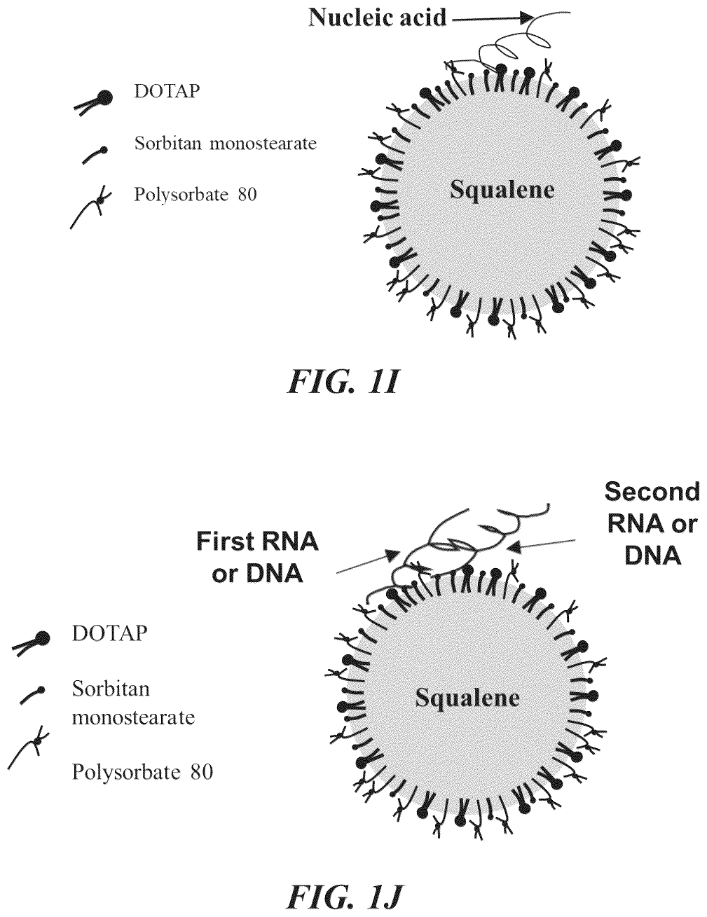

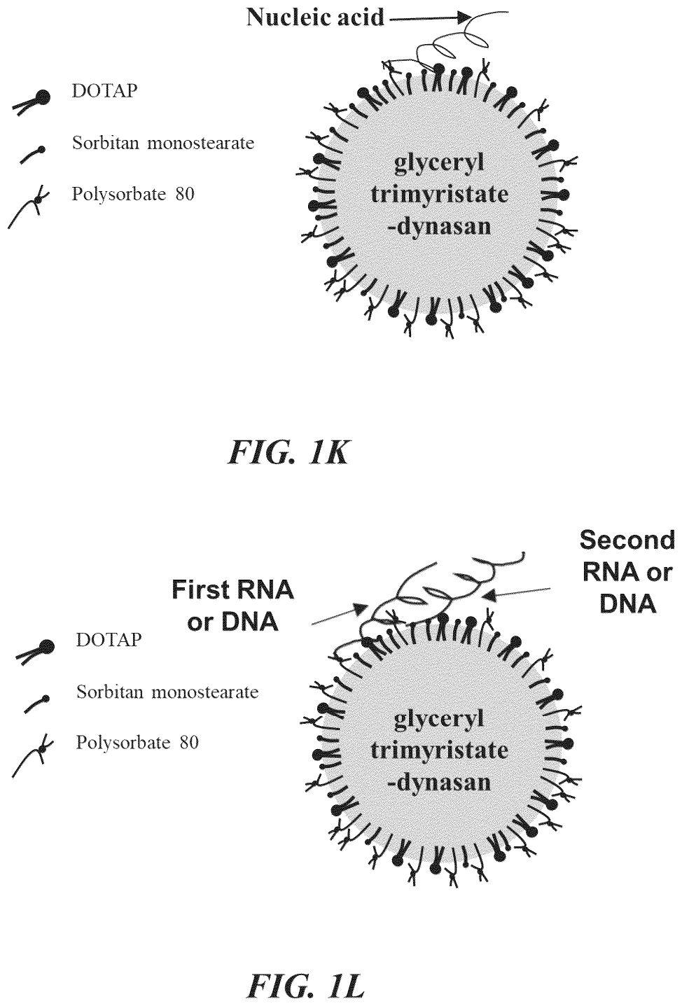

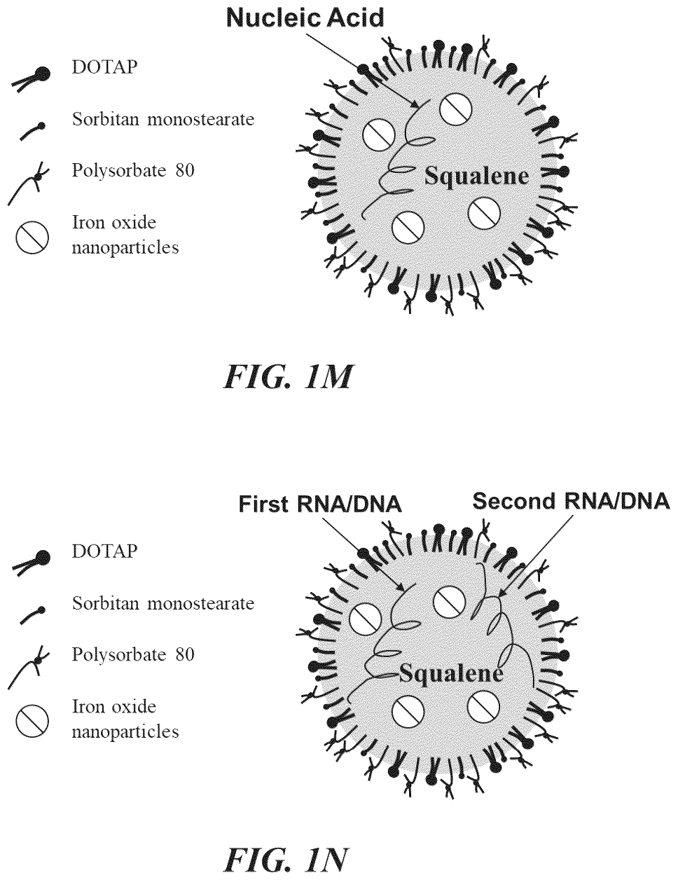

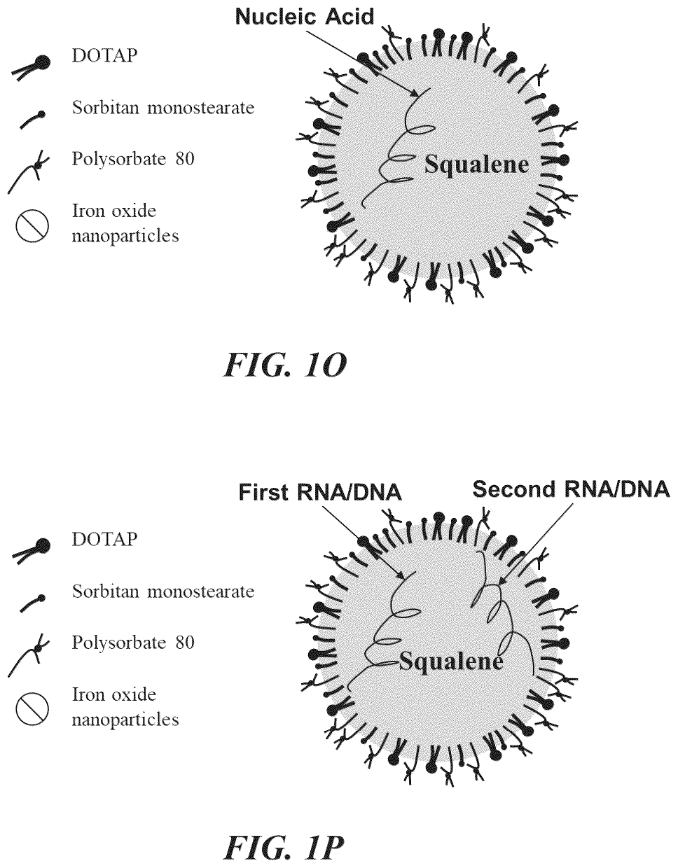

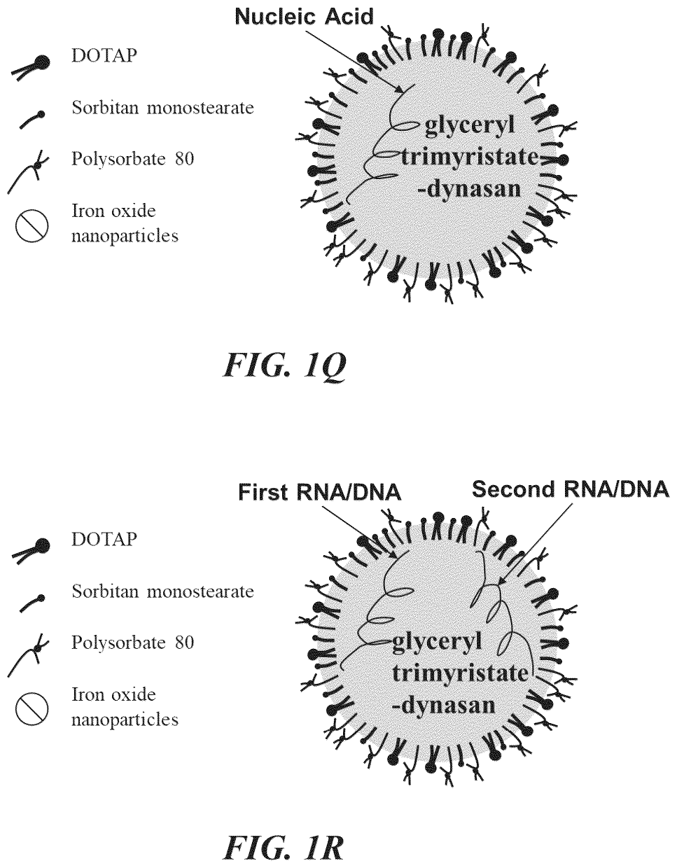

Various nanoparticles and formulations of nanoparticles (i.e., nanoemulsions) are employed. Exemplary nanoparticles are illustrated in FIGS. 1A-1R. Oil in water emulsions, as illustrated in FIG. 1A (not to scale), are stable, immiscible fluids containing an oil droplet dispersed in water or aqueous phase. FIG. 1B (not to scale) illustrates a nanostructured lipid carrier (NLCs) which can comprise a blend of solid organic lipids (e.g., trimyristin) and liquid oil (e.g., squalene). In NLCs, the solid lipid is dispersed in the liquid oil. The entire nanodroplet is dispersed in the aqueous (water) phase. In some embodiments, the nanoparticle comprises inorganic nanoparticles, as illustrated in FIG. 1C (not to scale), as solid inorganic nanoparticles (e.g., iron oxide nanoparticles) dispersed in liquid oil. FIG. 1D (not to scale) illustrates a nanoparticle comprising a cationic lipid membrane, a liquid oil, inorganic particles and a single nucleic acid, wherein the nucleic acid is in complex with the membrane. FIGS. 1I-1J illustrate a nanoparticle comprising a cationic lipid membrane (e.g., DOTAP), a liquid oil core (e.g., squalene) without an inorganic particle, and one or more nucleic acids, wherein the one or more nucleic acids are in complex with the membrane. In some embodiments, a nanoparticle provided herein comprises a solid core comprising glyceryl trimyristate-dynasan (FIGS. 1K-1L). FIG. 1D (not to scale) illustrates a nanoparticle comprising a cationic lipid membrane, a liquid oil, inorganic particles and nucleic acid in complex with the membrane. FIG. 1M (not to scale) illustrates a nanoparticle comprising a cationic lipid membrane, a liquid oil, inorganic particles and a single nucleic acid, wherein the nucleic acid is within the liquid core. FIGS. 1O-1P illustrate a nanoparticle comprising a cationic lipid membrane (e.g., DOTAP), a liquid oil core (e.g., squalene) without an inorganic particle, and one or more nucleic acids, wherein the one or more nucleic acids are within the liquid core. In some embodiments, a nanoparticle provided herein comprises a solid core comprising glyceryl trimyristate-dynasan (FIGS. 1Q-1R).

Nucleic acids provided herein can be complexed with a nanoparticle in Table 3 in cis (FIGS. 1A-1D, 1I, 1K, 1M, 1O, and 1Q) or in trans (FIGS. 1E-1H, 1J, 1L, 1N, 1P, and 1R). For example, a first RNA or DNA molecule can comprise a plurality of cancer-associated proteins and a second RNA or DNA molecule can comprise an RNA polymerase complex. As another example, a first RNA or DNA molecule can comprise one or more cancer-associated proteins and a RNA polymerase on the same nucleic acid; and a second RNA or DNA molecule can comprise an additional cancer-associated protein and/or an RNA polymerase.

Provided herein are nanoemulsions and nanodroplets comprising a plurality of lipid carriers or nanoparticles, wherein each lipid carrier or nanoparticle comprises a cationic lipid. In some embodiments, nanoemulsions comprises a plurality of cationic lipid carriers. In some embodiments, a composition provided herein comprises a cationic nanoemulsion. In some embodiments, cationic nanoemulsions described herein comprise a lipid (or other surfactant) molecules surrounding an oil particle that is dispersed in water and give the oil particle a cationic (positively charged) surface to which negatively-charged RNA molecules can adhere.

The entire nanodroplet can be dispersed as a colloid in the aqueous (water) phase or in a suspension. In some embodiments, nanoparticles provided herein are dispersed in an aqueous solution. Non-limiting examples of aqueous solutions include water (e.g., sterilized, distilled, deionized, ultra-pure, RNAse-free, etc.), saline solutions (e.g., Kreb's, Ascaris, Dent's, Tet's saline), or 1% (w/v) dimethyl sulfoxide (DMSO) in water.

In some embodiments, nanoparticles provided herein comprise a hydrophilic surface. In some embodiments, the hydrophilic surface comprises a cationic lipid. In some embodiments, the hydrophilic surface comprises an ionizable lipid. In some embodiments, the nanoparticle comprises a membrane. In some embodiments, the membrane comprises a cationic lipid. In some embodiments, the nanoparticles provided herein comprise a cationic lipid. Exemplary cationic lipids for inclusion in the hydrophilic surface include, without limitation: 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA), 1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FITS, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Other examples for suitable classes of lipids include, but are not limited to, the phosphatidylcholines (PCs), phosphatidylethanolamines (PEs), phosphatidylglycerol (PGs); and PEGylated lipids including PEGylated version of any of the above lipids (e.g., DSPE-PEGs). In some embodiments, the nanoparticle provided herein comprises DOTAP.

In some embodiments, the nanoparticle provided herein comprises an oil. In some embodiments, the oil is in liquid phase. Non-limiting examples of oils that can be used include α-tocopherol, coconut oil, dihydroisosqualene (DHIS), farnesene, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. In some embodiments, the nanoparticle provided herein comprises a triglyceride. Exemplary triglycerides include but are not limited to: capric triglycerides, caprylic triglycerides, a caprylic and capric triglycerides, triglyceride esters, and myristic acid triglycerins. In some embodiments, the hydrophobic lipid is in solid phase. In some embodiments, the hydrophobic lipid is in liquid phase, also referred to as an oil. In some embodiments, the hydrophobic lipid comprises squalene. In some embodiments, the hydrophobic lipid comprises solanesol.

In some embodiments, the nanoparticles provided herein comprise a liquid organic material and a solid inorganic material. In some embodiments, the nanoparticle provided herein comprises an inorganic particle. In some embodiments, the inorganic particle is a solid inorganic particle. In some embodiments, the nanoparticle provided herein comprises the inorganic particle within the hydrophobic core. In some embodiments, the nanoparticle provided herein comprises a metal. In some embodiments, the nanoparticle provided herein comprises a metal within the hydrophobic core. The metal can be without limitation, a metal salt, a metal oxide, a metal hydroxide, or a metal phosphate. In some embodiments, the nanoparticle provided herein comprises aluminum oxide (Al₂O₃), aluminum oxyhydroxide, iron oxide (Fe₃O₄, Fe₂O₃, FeO, or combinations thereof), titanium dioxide, silicon dioxide (SiO2), aluminum hydroxyphosphate (Al(OH)ₓ(PO₄)_{y}), calcium phosphate (Ca₃(PO₄)₂), calcium hydroxyapatite (Ca₁₀(PO₄)₆(OH)₂), iron gluconate, or iron sulfate. The inorganic particles may be formed from one or more same or different metals (any metals including transition metal). In some embodiments, the inorganic particle is a transition metal oxide. In some embodiments, the transition metal is magnetite (Fe₃O₄), maghemite (y-Fe₂O₃), wüstite (FeO), or hematite (alpha (α)-Fe₂O₃). In some embodiments, the metal is aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. In some embodiments, the metal can comprise a paramagnetic, a superparamagnetic, a ferrimagnetic or a ferromagnetic compound. In some embodiments, the metal is a superparamagnetic iron oxide (Fe₃O₄).

In some embodiments, nanoparticles provided herein comprise a cationic lipid, an oil, and optionally an inorganic particle. In some embodiments, nanoparticles provided herein comprise a cationic lipid, an oil, and an inorganic particle. In some embodiments, the nanoparticle provided herein comprises DOTAP; squalene and/or glyceryl trimyristate-dynasan; and iron oxide. In some embodiments, the nanoparticle provided herein further comprises a surfactant. Thus, in some embodiments, the nanoparticles provided herein comprise a cationic lipid, an oil, a surfactant, and optionally an inorganic particle. In some embodiments, the nanoparticles provided herein comprise a cationic lipid, an oil, an inorganic particle, and a surfactant.

Surfactants are compounds that lower the surface tension between two liquids or between a liquid and a solid component of the nanoparticles provided herein. Surfactants can be hydrophobic, hydrophilic, or amphiphilic. In some embodiments, the nanoparticle provided herein comprises a hydrophobic surfactant. Exemplary hydrophobic surfactants that can be employed include but are not limited to: sorbitan monolaurate (SPAN® 20), sorbitan monopalmitate (SPAN® 40), sorbitan monostearate (SPAN® 60), sorbitan tristearate (SPAN® 65), sorbitan monooleate (SPAN® 80), and sorbitan trioleate (SPAN® 85).

Suitable hydrophobic surfactants include those having a hydrophilic-lipophilic balance (HLB) value of 10 or less, for instance, 5 or less, from 1 to 5, or from 4 to 5. For instance, the hydrophobic surfactant can be a sorbitan ester having an HLB value from 1 to 5, or from 4 to 5. In some embodiments, nanoparticles provided herein comprise a ratio of the esters that yields a hydrophilic-lipophilic balance between 8 and 11. HLB is used to categorize surfactants as hydrophilic or lipophilic. The HLB scale provides for the classification of surfactant function calculated e.g., by Griffin's method:

$$HLB = \frac{20 {M}_{h}}{M} .$$

where Mₕ is the molecular mass of the hydrophilic portion of the lipid carrier and M is the molecular mass of the lipid carrier. The HLB scale is provided below:
HLB=0: fully lipophilic/hydrophobic carrier;
HLB between 0 and 6 is an oil soluble carrier;
HLB between 6 and 9 is a water dispersible carrier;
HLB between 9 and 20 is a hydrophilic, water soluble carrier;
HLB=20: fully hydrophilic/lipophobic carrier.

In some embodiments, a nanoparticle or a lipid carrier provided herein comprises a hydrophilic surfactant, also called an emulsifier. In some embodiments, a nanoparticle or a lipid carrier provided herein comprises polysorbate. Polysorbates are oily liquids derived from ethoxylated sorbitan (a derivative of sorbitol) esterified with fatty acids. Exemplary hydrophilic surfactants that can be employed include but are not limited to: polysorbates such as TWEEN®, Kolliphor, Scattics, Alkest, or Canarcel; polyoxyethylene sorbitan ester (polysorbate); polysorbate 80 (polyoxyethylene sorbitan monooleate, or TWEEN® 80); polysorbate 60 (polyoxyethylene sorbitan monostearate, or TWEEN® 60); polysorbate 40 (polyoxyethylene sorbitan monopalmitate, or TWEEN® 40); and polysorbate 20 (polyoxyethylene sorbitan monolaurate, or TWEEN® 20). In one embodiment, the hydrophilic surfactant is polysorbate 80.

Nanoparticles provided herein comprise a hydrophobic core surrounded by a lipid membrane (e.g., a cationic lipid such as DOTAP). In some embodiments, the hydrophobic core comprises: a phosphate-terminated lipid; and a surfactant. In some embodiments, the hydrophobic core comprises: one or more inorganic particles; a phosphate-terminated lipid; and a surfactant.

Inorganic solid nanoparticles described herein can be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. In some embodiments, the inorganic particle is coated with a capping ligand, the phosphate-terminated lipid, and/or the surfactant. In some embodiments the hydrophobic core comprises a phosphate-terminated lipid. Exemplary phosphate-terminated lipids that can be employed include but are not limited to: trioctylphosphine oxide (TOPO) or distearyl phosphatidic acid (DSPA). In some embodiments, the hydrophobic core comprises a surfactant such as a phosphorous-terminated surfactant, a carboxylate-terminated surfactant, a sulfate-terminated surfactant, or an amine-terminated surfactant. Exemplary carboxylate-terminated surfactants include oleic acid. Typical amine terminated surfactants include oleylamine. In some embodiments, the surfactant is distearyl phosphatidic acid (DSPA), oleic acid, oleylamine or sodium dodecyl sulfate (SDS). In some embodiments, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.

In some embodiments, the hydrophobic core comprises: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core comprises: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80.

In some embodiments, the hydrophobic core consists of: one or more inorganic particles containing at least one metal hydroxide or oxyhydroxide particle optionally coated with a phosphate-terminated lipid, a phosphorous-terminated surfactant, a carboxylate-terminated surfactant, a sulfate-terminated surfactant, or an amine-terminated surfactant; and a liquid oil containing naturally occurring or synthetic squalene; a cationic lipid comprising DOTAP; a hydrophobic surfactant comprising a sorbitan ester selected from the group consisting of: sorbitan monostearate, sorbitan monooleate, and sorbitan trioleate; and a hydrophilic surfactant comprising a polysorbate.

In some embodiments, the hydrophobic core consists of: one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In some embodiments, the nanoparticle provided herein can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In some embodiments the nanoparticle provided herein from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80.

In some embodiments, the nanoparticle provided herein can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80.

In some embodiments, the nanoparticle provided herein can comprise from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80.

In some embodiments, a composition described herein comprises at least one nanoparticle formulation as described in Table 3. In some embodiments, a composition described herein comprises any one of NP-1 to NP-30. In some embodiments, a composition described herein comprises any one of NP-1 to NP-37. In some embodiments, the nanoparticles provided herein are admixed with a nucleic acid provided herein. In some embodiments, nanoparticles provided herein are made by homogenization and ultrasonication techniques.

TABLE 3
Nanoparticle Formulations.

| | Cationic Lipid(s) | Oil(s) | Surfactant(s) | Additional Ingredients |
|---|---|---|---|---|
| Name | % (w/v) or mg/ml | % (w/v) or mg/ml | % (w/v) or mg/ml | % (w/v), mg/ml, or mM |
| NP-1 | 30 mg/ml 1,2-dioleoyl-3- | 37.5 mg/ml squalene | 37 mg/ml sorbitan | 0.2 mg Fe/ml 12 nm oleic |
| | trimethylammonium- | | monostearate, (2R)- | acid-coated iron oxide |
| | propane (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate. |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-2 | 30 mg/ml 1,2-dioleoyl-3- | 37.5 mg/ml squalene | 37 mg/ml sorbitan | 1 mg Fe/ml 15 nm oleic |
| | trimethylammonium- | | monostearate (2R)- | acid-coated iron oxide |
| | propane (DOTAP) chloride | | 2-[(2R,3R,4S)-3,4- | nanoparticles |
| | | | Dihydroxyoxolan-2- | 10 mM sodium citrate |
| | | | yl]-2-hydroxyethyl | dihydrate |
| | | | octadecenoate | |
| | | | C₂₄H₄₆O₆ | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-3 | 30 mg/ml 1,2-dioleoyl-3- | 37.5 mg/ml Miglyol | 37 mg/ml sorbitan | 0.2 mg Fe/ml 15 nm oleic |
| | trimethylammonium- | 812 N | monostearate, (2R)- | acid-coated iron oxide |
| | propane (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate |
| | | oil derived caprylic | octadecenoate | |
| | | and capric fatty acids | C₂₄H₄₆O₆ | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-4 | 30 mg/ml 1,2-dioleoyl-3- | 37.5 mg/ml Miglyol | 37 mg/ml sorbitan | 1 mg Fe/ml 15 nm oleic |
| | trimethylammonium- | 812 N | monostearate, (2R)- | acid-coated iron oxide |
| | propane (DOTAP) chloride | (triglyceride ester of | 2-[(2R,3R,4S)-3,4- | nanoparticles |
| | | saturated | Dihydroxyoxolan-2- | 10 mM sodium citrate |
| | | coconut/palmkernel | yl]-2-hydroxyethyl | dihydrate. |
| | | oil derived caprylic | octadecenoate, | |
| | | and capric fatty acids | C₂₄H₄₆O₆) | |
| | | and plant derived | (SPAN ® 60) | |
| | | glycerol) | 37 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-5 | 30 mg/ml DOTAP chloride | 37.5 mg/ml squalene | 37 mg/ml sorbitan | 1 mg/ml trioctylphosphine |
| | | | monostearate | oxide (TOPO)-coated |
| | | | (SPAN ® 60) | aluminum hydroxide |
| | | | 37 mg/ml | (Alhydrogel ® 2%) |
| | | | polysorbate 80 | particles |
| | | | (TWEEN ® 80) | 10 mM sodium citrate |
| | | | | dihydrate |
| NP-6 | 30 mg/ml DOTAP chloride | 37.5 mg/ml Solaneso | 37 mg/ml sorbitan | 0.2 mg Fe/ml oleic acid- |
| | | (Cayman chemicals) | monostearate | coated iron oxide |
| | | | (SPAN ® 60) | nanoparticles |
| | | | 37 mg/ml | 10 mM sodium citrate |
| | | | polysorbate 80 | |
| | | | (TWEEN ® 80) | |
| NP-7 | 30 mg/ml DOTAP chloride | 37.5 mg/ml squalene | 37 mg/ml sorbitan | 10 mM sodium citrate |
| | | 2.4 mg/ml Dynasan | monostearate | |
| | | 114 | (SPAN ® 60) | |
| | | | 37 mg/ml | |
| | | | polysorbate 80 | |
| | | | (TWEEN ® 80) | |
| NP-8 | 4 mg/ml DOTAP chloride | 43 mg/ml squalene | 5 mg/ml sorbitan | 10 mM sodium citrate |
| | | | trioleate (SPAN ® | |
| | | | 85) | |
| | | | 5 mg/ml polysorbate | |
| | | | 80 (TWEEN ® 80) | |
| NP-9 | 7.5 mg/ml 1,2-dioleoyl-3- | 9.4 mg/ml squalene | 9.3 mg/ml sorbitan | 0.05 mg/ml 15 nanometer |
| | trimethylammonium- | ((6E,10E,14E,18E)- | monostearate (2R)- | superparamagnetic iron |
| | propane (DOTAP) chloride | 2,6,10,15,19,23- | 2-[(2R,3R,4S)-3,4- | oxide (Fe₃O₄) |
| | | Hexamethyltetracosa- | Dihydroxyoxolan-2- | 10 mM sodium citrate |
| | | 2,6,10,14,18,22- | yl]-2-hydroxyethyl | dihydrate |
| | | hexaene, C₃₀H₅₀) | octadecenoate, | |
| | | 0.63 mg/ml glyceryl | C₂₄H₄₆₀O₆) | |
| | | trimyristate-dynasan | (SPAN ® 60) | |
| | | (DYNASAN 114 ®) | 9.3 mg/ml | |
| | | | polyoxyethylene | |
| | | | (20) sorbitan | |
| | | | monooleate, | |
| | | | C₆₄H₁₂₄O₂₆, | |
| | | | Polysorbate 80 | |
| | | | (TWEEN ® 80) | |
| NP-10 | 0.4% DOTAP | 0.25% glyceryl | 0.5% sorbitan | |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-11 | 3.0% DOTAP | 0.25% glyceryl | 3.7% sorbitan | |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-12 | 0.4% DOTAP | 4.3% Squalene | 0.5% sorbitan | |
| | | | trioleate (SPAN ® | |
| | | | 85) | |
| | | | 0.5% polysorbate 80 | |
| | | | (TWEEN ® 80) | |
| NP-13 | 0.4% DOTAP | 0.25% glyceryl | 2.0% polysorbate 80 | |
| | | trimyristate-dynasan | (TWEEN ® 80) | |
| | | (DYNASAN 114 ®) | | |
| | | 4.08% squalene | | |
| NP-14 | 0.4% DOTAP | 0.25% glyceryl | 0.5% sorbitan | |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-15 | 0.4% DOTAP | 0.25% glyceryl | 0.25% sorbitan | |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-16 | 0.4% DOTAP | 5% squalene | 0.5% sorbitan | |
| | | | trioleate (SPAN ® | |
| | | | 85) | |
| | | | 2.0% polysorbate 80 | |
| | | | (TWEEN ® 80) | |
| NP-17 | 0.4% DOTAP | 5% squalene | 0.5% sorbitan | |
| | | | monostearate | |
| | | | (SPAN ® 60) | |
| | | | 2% polysorbate 80 | |
| | | | (TWEEN ® 80) | |
| NP-18 | 0.4% DOTAP | 0.25% glyceryl | 2% sorbitan trioleate | |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-19 | 0.4% DOTAP | 0.25% glyceryl | 0.5% sorbitan | 1% aluminum hydroxide |
| | | trimyristate-dynasan | monostearate | |
| | | (DYNASAN 114 ®) | (SPAN ® 60) | |
| | | 4.75% Squalene | 0.5% polysorbate 80 | |
| | | | (TWEEN ® 80) | |
| NP-20 | 3.0% DOTAP | 0.25% glyceryl | 3.7% sorbitan | 1% aluminum hydroxide |
| | | trimyristate-dynasan | monostearate | |
| | | (DYNASAN 114 ®) | (SPAN ® 60) | |
| | | 3.75% Squalene | 3.7% polysorbate 80 | |
| | | | (TWEEN ® 80) | |
| NP-21 | 0.4% DOTAP | 4.3% Squalene | 0.5% sorbitan | 1% aluminum hydroxide |
| | | | trioleate (SPAN ® | |
| | | | 85) | |
| | | | 0.5% polysorbate 80 | |
| | | | (TWEEN ® 80 | |
| NP-22 | 0.4% DOTAP | 0.25% glyceryl | 2.0% polysorbate 80 | 1% aluminum hydroxide |
| | | trimyristate-dynasan | (TWEEN ® 80) | |
| | | (DYNASAN 114 ®) | | |
| | | 4.08% squalene | | |
| NP-23 | 0.4% DOTAP | 0.25% glyceryl | 0.5% sorbitan | 1% aluminum hydroxide |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80 | |
| NP-24 | 0.4% DOTAP | 0.25% glyceryl | 0.25% sorbitan | 1% aluminum hydroxide |
| | | trimyristate-dynasan | trioleate (SPAN ® | |
| | | (DYNASAN 114 ®) | 85) | |
| | | 4.08% squalene | 2.0% polysorbate 80 | |
| | | | (TWEEN ® 80) | |
| NP-25 | 0.4% DOTAP | 5% squalene | 0.5% sorbitan | 1% aluminum hydroxide |
| | | | trioleate (SPAN ® | |
| | | | 85) | |
| | | | 2.0% polysorbate 80 | |
| | | | (TWEEN ® 80) | |
| NP-26 | 0.4% DOTAP | 5% squalene | 0.5% sorbitan | 1% aluminum hydroxide |
| | | | monostearate | |
| | | | (SPAN ® 60) | |
| | | | 2% polysorbate 80 | |
| | | | (TWEEN ® 80) | |
| NP-27 | 0.4% DOTAP | 0.25% glyceryl | 2% sorbitan trioleate | 1% aluminum hydroxide |
| | | trimyristate-dynasan | (SPAN ® 85) | |
| | | (DYNASAN 114 ®) | 2% polysorbate 80 | |
| | | 4.08% squalene | (TWEEN ® 80) | |
| NP-28 | 0.5-5.0 mg/ml DOTAP | 0.2-10% (v/v) | 0.01-2.5% (v/v) | |
| | | squalene | polysorbate 80 | |
| | | | (TWEEN ® 80) | |
| NP-29 | 0.4% (w/w) DOTAP | 4.3% (w/w) squalene | 0.5% (w/w) sorbitan | |
| | | | trioleate (SPAN ® | |
| | | | 85) | |
| | | | 0.5% (w/w) | |
| | | | polysorbate 80 | |
| | | | (TWEEN ® 80) | |
| NP-30 | 30 mg/ml DOTAP chloride | 37.5 mg/ml squalene | 37 mg/ml sorbitan | 10 mM sodium citrate |
| | | | monostearate | |
| | | | (SPAN ® 60) | |
| | | | 37 mg/ml | |
| | | | polysorbate 80 | |
| | | | (TWEEN ® 80) | |
| NP-31 | 30 mg/ml DOTAP chloride | 37.5 mg/ml squalene | 37 mg/ml sorbitan | 0.4 mg Fe/ml 5 nm oleic |
| | | | monostearate | acid-coated iron oxide |
| | | | (SPAN ® 60) | nanoparticles |
| | | | 37 mg/ml | 10 mM sodium citrate |
| | | | polysorbate 80 | dihydrate |
| | | | (TWEEN ® 80) | |
| NP-32 | 0.8-1.6 mg/ml DOTAP | 4.5% squalene | 0.5% (w/w) | 10 mM sodium citrate |
| | chloride | | sorbitan trioleate | |
| | | | (SPAN 85 ®) | |
| | | | 0.5% (w/w) | |
| | | | polysorbate 80 | |
| | | | (TWEEN ® 80) | |
| NP-33 | 45-55 mol % ionizable | 35-42 mol % | 1.25-1.75 mol % | |
| | cationic lipid | cholesterol | PEG2000-DMG | |
| | 8-12 mol % | | | |
| | distearoylphosphatidylcholine | | | |
| | (DSPC) | | | |
| NP-34 | 50 mol % D-Lin-MC3-DMA | 38.5% cholesterol | 1.5% PEG-lipid | |
| | (MC3) | | | |
| | 10 mol % | | | |
| | distearoylphosphatidylcholine | | | |
| | (DSPC) | | | |
| NP-35 | 50 mol % Lipid H (SM-102) | 38.5% cholesterol | 1.5 mol % | |
| | 10 mol % | | PEG2000-DMG | |
| | distearoylphosphatidylcholine | | | |
| | (DSPC) | | | |
| NP-36 | 30 mg/ml 1,2-dioleoyl-3- | 3.75% w/v glyceryl | 37 mg/ml sorbitan | 10 mM sodium citrate |
| | trimethylammonium- | trimyristate-dynasan | monostearate, (2R)- | dihydrate |
| | propane (DOTAP) chloride | (DYNASAN 114 ®) | 2-[(2R,3R,4S)-3,4- | |
| | | | Dihydroxyoxolan-2- | |
| | | | yl]-2-hydroxyethyl | |
| | | | octadecenoate, | |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |
| NP-37 | 30 mg/ml 1,2-dioleoyl-3- | 3.75% w/v glyceryl | 37 mg/ml sorbitan | 0.2 mg Fe/mL or 0.02% |
| | trimethylammonium- | trimyristate-dynasan | monostearate, (2R)- | wFe/v of 5 to 15 nm |
| | propane (DOTAP) chloride | (DYNASAN 114 ®) | 2-[(2R,3R,4S)-3,4- | diameter iron oxide |
| | | | Dihydroxyoxolan-2- | nanoparticles |
| | | | yl]-2-hydroxyethyl | 10 mM sodium citrate |
| | | | octadecenoate, | dihydrate |
| | | | C₂₄H₄₆O₆) | |
| | | | (SPAN ® 60) | |
| | | | 37 mg/ml | |
| | | | polyoxyethylene | |
| | | | (20) sorbitan | |
| | | | monooleate, | |
| | | | C₆₄H₁₂₄O₂₆ | |
| | | | Polysorbate 80 | |
| | | | (TWEEN ® 80) | |

In some embodiments, nanoparticles provided herein comprise: sorbitan monostearate (e.g., SPANS® 60), polysorbate 80 (e.g., TWEEN® 80), DOTAP, squalene, and no solid particles. In some embodiments, nanoparticles provided herein comprise: sorbitan monostearate (e.g., SPANS® 60), polysorbate 80 (e.g., TWEEN® 80), DOTAP, squalene, and iron oxide particles. In some embodiments, nanoparticles provided herein comprise an immune stimulant. In some embodiments, the immune stimulant is squalene. In some embodiments, the immune stimulant is Miglyol 810 or Miglyol 812. Miglyol 810 is a triglyceride ester of saturated caprylic and capric fatty acids and glycerol. Miglyol 812 is a triglyceride ester of saturated coconut/palm kernel oil derived caprylic and capric fatty acids and plant derived glycerol. In some embodiments, the immune stimulant can decrease the total amount of protein produced, but can increase the immune response to a composition provided herein (e.g., when delivered as a vaccine). In some embodiments, the immune stimulant can increase the total amount of protein produced, but can decrease the immune response to a composition provided herein.

Nanoparticles provided herein can be of various average diameters in size. In some embodiments, nanoparticles provided herein have an average diameter (z-average hydrodynamic diameter, measured by dynamic light scattering) ranging from about 20 nm to about 200 nm. In some embodiments, the z-average diameter of the nanoparticle ranges from about 20 nm to about 150 nm, from about 20 nm to about 100 nm, from about 20 nm to about 80 nm, from about 20 nm to about 60 nm. In some embodiments, the z-average diameter of the nanoparticle ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the nanoparticle is from about 40 nm to about 80 nm. In some embodiments, the z-average diameter of the nanoparticle is from about 40 nm to about 60 nm. In some embodiments, the nanoparticle is up to 100 nm in diameter. In some embodiments, the nanoparticle is 50 to 70 nm in diameter. In some embodiments, the nanoparticle is 40 to 80 nm in diameter. In some embodiments, a nanoparticle provided herein comprises an inorganic particle, wherein the inorganic particle is within the hydrophobic core of the nanoparticle. In some embodiments, the inorganic particle can be an average diameter (number weighted average diameter) ranging from about 3 nm to about 50 nm. For instance, the inorganic particle can have an average diameter of about 5 nm, about 10 nm, about 15 nm, about 20 nm, about 25 nm, about 30 nm, about 35 nm, about 40 nm, about 45 nm, or about 50 nm. In some embodiments, the ratio of esters and lipids yield a particle size between 30 nm and 200 nm. In some embodiments, the ratio of esters and lipids yield a particle size between 40 nm and 70 nm.

Nanoparticles provided herein may be characterized by the polydispersity index (PDI), which is an indication of their quality with respect to size distribution. In some embodiments, the average polydispersity index (PDI) of the nanoparticles provided herein ranges from about 0.1 to about 0.5. In some embodiments, the average PDI of the nanoparticles can range from about 0.2 to about 0.5, from about 0.1 to about 0.4, from about 0.2 to about 0.4, from about 0.2 to about 0.3, or from about 0.1 to about 0.3.

In some embodiments, nanoparticles provided herein comprise an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1.

In some embodiments, nanoparticles provided herein comprise a hydrophilic surfactant-to-lipid ratio ranging from about 0.1:1 to about 2:1, from about 0.2:1 to about 1.5:1, from about 0.3:1 to about 1:1, from about 0.5:1 to about 1:1, or from about 0.6:1 to about 1:1. In some embodiments, the nanoparticles provided herein comprise a hydrophobic surfactant-to-lipid ratio ranging from about 0.1:1 to about 5:1, from about 0.2:1 to about 3:1, from about 0.3:1 to about 2:1, from about 0.5:1 to about 2:1, or from about 1:1 to about 2:1.

In some embodiments, the nanoparticles provided herein comprise from about 0.2% to about 40% w/v liquid oil, from about 0.001% to about 10% w/v inorganic solid nanoparticle, from about 0.2% to about 10% w/v lipid, from about 0.25% to about 5% w/v hydrophobic surfactant, and from about 0.5% to about 10% w/v hydrophilic surfactant. In some embodiments, the lipid comprises a cationic lipid, and the oil comprises squalene, and/or the hydrophobic surfactant comprises sorbitan ester.

### Combination Compositions

Provided herein are compositions comprising a nanoparticle described herein and a nucleic acid encoding for a cancer-associated protein, or cancer-associated protein binding protein. In some embodiments, nucleic acids provided herein are incorporated, associated with, or complexed a lipid carrier provided herein to form a lipid carrier-nucleic acid complex. The lipid carrier-nucleic acid complex is formed via non-covalent interactions or via reversible covalent interactions.

Further provided herein is a nanoemulsion comprising a plurality of nanoparticles provided herein. In some embodiments, the nucleic acid further encodes for an RNA-dependent polymerase. In some embodiments, the RNA-dependent polymerase is a viral RNA polymerase. In some embodiments, the nucleic acid encoding for the RNA polymerase is on the same nucleic acid strand as the nucleic acid sequence encoding for the protein (e.g., cis). In some embodiments, the nucleic acid encoding for the RNA polymerase is on a different nucleic acid strand as the nucleic acid sequence encoding for the protein (e.g., trans). In some embodiments, the nucleic acid encoding for the RNA polymerase is a DNA molecule. In some embodiments, nucleic acid sequences encoding for a cancer-associated protein, a tumor antigen, a neoantigen, a cancer therapeutic antibody, or a functional fragment thereof are DNA or RNA molecules. In some embodiments, cancer-associated proteins and cancer therapeutic antibodies provided herein are encoded by DNA. Nanoparticles for inclusion include, without limitation, any one of NP-1 to NP-31, or any one of NP-1 to NP-37. Nucleic acids for inclusion include, without limitation, comprise a region comprising any one of, or a plurality of, SEQ ID NOS: 8, 12-17, and/or encodes for an amino acid sequence set forth in any one of SEQ ID NOS: 1-7, 9-11. In some instances, the nucleic acids further comprise a region encoding for an RNA polymerase, e.g., a region comprising a sequence of SEQ ID NO: 8.

Compositions provided herein can be characterized by an nitrogen:phosphate (N:P) molar ratio. The N:P ratio is determined by the amount of cationic lipid in the nanoparticle which contain nitrogen and the amount of nucleic acid used in the composition which contain negatively charged phosphates. A molar ratio of the lipid carrier to the nucleic acid can be chosen to increase the delivery efficiency of the nucleic acid, increase the ability of the nucleic acid-carrying nanoemulsion composition to elicit an immune response to the antigen, increase the ability of the nucleic acid-carrying nanoemulsion composition to elicit the production of antibody titers to the antigen in a subject. In some embodiments, compositions provided herein have a molar ratio of the lipid carrier to the nucleic acid can be characterized by the nitrogen-to-phosphate molar ratio, which can range from about 0.01:1 to about 1000:1, for instance, from about 0.2:1 to about 500:1, from about 0.5:1 to about 150:1, from about 1:1 to about 150:1, from about 1:1 to about 125:1, from about 1:1 to about 100:1, from about 1:1 to about 50:1, from about 1:1 to about 50:1, from about 5:1 to about 50:1, from about 5:1 to about 25:1, or from about 10:1 to about 20:1. In certain embodiments, the molar ratio of the lipid carrier to the nucleic acid, characterized by the nitrogen-to-phosphate (N:P) molar ratio, ranges from about 1:1 to about 150:1, from about 5:1 to about 25:1, or from about 10:1 to about 20:1. In one embodiment, the N:P molar ratio of the nanoemulsion composition is about 15:1. In some embodiments, the nanoparticle comprises a nucleic acid provided herein covalently attached to the membrane.

Compositions provided herein can be characterized by an oil-to-surfactant molar ratio. In some embodiments, the oil-to-surfactant ratio is the molar ratio of squalene: DOTAP, hydrophobic surfactant, and hydrophilic surfactant. In some embodiments, the oil-to-surfactant ratio is the molar ratio of squalene: DOTAP, sorbitan monostearate, and polysorbate 80. In some embodiments, the oil-to surfactant molar ratio ranges from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. In some embodiments, the oil-to-surfactant molar ratio is at least about 0.1:1, at least about 0.2:1, at least about 0.3:1, at least about 0.4:1, at least about 0.5:1, at least about 0.6:1, at least about 0.7:1. In some embodiments, the oil-to surfactant molar ratio is at least about 0.4:1 up to 1:1.

Compositions provided herein can be characterized by hydrophilic surfactant-to-lipid (e.g., cationic lipid) ratio. In some embodiments, the hydrophilic surfactant-to-lipid ratio ranges from about 0.1:1 to about 2:1, from about 0.2:1 to about 1.5:1, from about 0.3:1 to about 1:1, from about 0.5:1 to about 1:1, or from about 0.6:1 to about 1:1. Compositions provided herein can be characterized by hydrophobic surfactant-to-lipid (e.g., cationic lipid) ratio ranging. In some embodiments, the hydrophobic surfactant-to-lipid ratio ranges from about 0.1:1 to about 5:1, from about 0.2:1 to about 3:1, from about 0.3:1 to about 2:1, from about 0.5:1 to about 2:1, or from about 1:1 to about 2:1.

Provided herein is a dried composition comprising a sorbitan fatty acid ester, an ethoxylated sorbitan ester, a cationic lipid, an immune stimulant, and an RNA. Further provided herein are dried compositions, wherein the dried composition comprises sorbitan monostearate (e.g., SPAN® 60), polysorbate 80 (e.g., TWEEN® 80), DOTAP, and an RNA.

### Thermally Stable, Dried, and Lyophilized Vaccines

Provided herein are dried or lyophilized compositions and vaccines. Further provided herein are pharmaceutical compositions comprising a dried or lyophilized composition provided herein that is reconstituted in a suitable diluent and a pharmaceutically acceptable carrier. In some embodiments, the diluent is aqueous. In some embodiments, the diluent is water.

A lyophilized composition is generated by a low temperature dehydration process involving the freezing of the composition, followed by a lowering of pressure, and removal of ice by sublimation. In certain cases, lyophilization also involves the removal of bound water molecules through a desorption process. In some embodiments, compositions and vaccine compositions provided herein are spray-dried. Spray drying is a process by which a solution is fed through an atomizer to create a spray, which is thereafter exposed to a heated gas stream to promote rapid evaporation. When sufficient liquid mass has evaporated, the remaining solid material in the droplet forms particles which are then separated from the gas stream (e.g., using a filter or a cyclone). Drying aids in the storage of the compositions and vaccine compositions provided herein at higher temperatures (e.g., greater than 4° C.) as compared to the sub-zero temperatures needed for the storage of existing mRNA vaccines. In some embodiments, dried compositions and lyophilized compositions provided herein comprise (a) a lipid carrier, wherein the lipid carrier is a nanoemulsion comprising: (i) a hydrophobic core; (ii) optionally, one or more inorganic nanoparticles; (iii) and one or more lipids; (b) one or more nucleic acids; and (c) at least one cryoprotectant. In some embodiments, the cryoprotectant is selected from the group consisting of: sucrose, maltose, trehalose, mannitol, glucose, and any combinations thereof. Additional examples of cryoprotectants include but are not limited to: dimethyl sulfoxide (DMSO), glycerol, propylene glycol, ethylene glycol, 3-O-methyl-D-glucopyranose (3-OMG), olyethylene glycol (PEG), 1,2-propanediol, acetamide, trehalose, formamide, sugars, proteins, and carbohydrates.

In some embodiments, compositions and methods provided herein comprise at least one cryoprotectant. Exemplary cryoprotectants for inclusion are, but not limited to, sucrose, maltose, trehalose, mannitol, or glucose, and any combinations thereof. In some embodiments, additional or alternative cryoprotectant for inclusion is sorbitol, ribitol, erthritol, threitol, ethylene glycol, or fructose. In some embodiments, additional or alternative cryoprotectant for inclusion is dimethyl sulfoxide (DMSO), glycerol, propylene glycol, ethylene glycol, 3-O-methyl-D-glucopyranose (3-OMG), polyethylene glycol (PEG), 1,2-propanediol, acetamide, trehalose, formamide, sugars, proteins, and carbohydrates. In some embodiments, the cryoprotectant is present at about 1% w/v to at about 20% w/v, preferably about 10% w/v to at about 20% w/v, and more preferably at about 10% w/v. In certain aspects of the disclosure, the cryoprotectant is sucrose. In some aspects of the disclosure, the cryoprotectant is maltose. In some aspects of the disclosure, the cryoprotectant is trehalose. In some aspects of the disclosure, the cryoprotectant is mannitol. In some aspects of the disclosure, the cryoprotectant is glucose. In some embodiments, the cryoprotectant is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the cryoprotectant is present in an amount of about 50 to about 500 mg. In some embodiments, the cryoprotectant is present in an amount of about 200 to about 300 mg. In some embodiments, the cryoprotectant is present in an amount of about 250 mg. In some embodiments, the cryoprotectant is present in amount of a lyophilized composition by weight of at least about 50, 55, 60, 65, 70, 75, 80, 85, 90, 95 or more percent. In some embodiments, the cryoprotectant is present in amount of a lyophilized composition by weight of about 95%. In some embodiments, the cryoprotectant is present in amount of a lyophilized composition by weight of 80 to 98%, 85 to 98%, 90 to 98%, or 94 to 96%. In some embodiments, the cryoprotectant is a sugar. In some embodiments, the sugar is sucrose, maltose, trehalose, mannitol, or glucose. In some embodiments, the sugar is sucrose. In some embodiments, the sucrose is present in an amount of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 325, 350, 375, 400, 450, 500 or more mg. In some embodiments, the sucrose is present in an amount of about 50 to about 500 mg. In some embodiments, the sucrose is present in an amount of about 200 to about 300 mg. In some embodiments, the sucrose is present in an amount of about 250 mg. In some embodiments, the sucrose is present in amount of a lyophilized composition by weight of at least about 50, 55, 60, 65, 70, 75, 80, 85, 90, 95 or more percent. In some embodiments, the sucrose is present in amount of a lyophilized composition by weight of about 95%. In some embodiments, the sucrose is present in amount of a lyophilized composition by weight of 80 to 98%, 85 to 98%, 90 to 98%, or 94 to 96%.

In some embodiments, the cryoprotectant is sucrose. In some embodiments, the cryoprotectant is at a concentration of at least about 0.1% w/v. In some embodiments, the cryoprotectant is at a concentration of about 1% w/v to at about 20% w/v. In some embodiments, the cryoprotectant is at a concentration of about 10% w/v to at about 20% w/v. In some embodiments, the cryoprotectant is at a concentration of about 10% w/v.

In some embodiments, compositions and vaccine compositions provided herein are thermally stable. A composition is considered thermally stable when the composition resists the action of heat or cold and maintains its properties, such as the ability to protect a nucleic acid molecule from degradation at given temperature. In some embodiments, compositions and vaccine compositions provided herein are thermally stable at about 25 degrees Celsius (° C.) or standard room temperature. In some embodiments, compositions and vaccine compositions provided herein are thermally stable at about 45° C. In some embodiments, compositions and vaccine compositions provided herein are thermally stable at about −20° C. In some embodiments, compositions and vaccine compositions provided herein are thermally stable at about 2° C. to about 8° C. In some embodiments, compositions and vaccine compositions provided herein are thermally stable at a temperature of at least about −80° C., at least about −20° C., at least about 0° C., at least about 2° C., at least about 4° C., at least about 6° C., at least about 8° C., at least about 10° C., at least about 20° C., at least about 25° C., at least about 30° C., at least about 37° C., up to 45° C. In some embodiments, compositions and vaccine compositions provided herein are thermally stable for at least about 5 day, at least about 1 week, at least about 2 weeks, at least about 1 month, up to 3 months. In some embodiments, compositions and vaccine compositions provided herein are stored at a temperature of at least about 4° C. up to 37° C. for at least about 5 day, at least about 1 week, at least about 2 weeks, at least about 1 month, up to 3 months. In some embodiments, compositions and vaccine compositions provided herein are stored at a temperature of at least about 20° C. up to 25° C. for at least about 5 day, at least about 1 week, at least about 2 weeks, at least about 1 month, up to 3 months.

Also provided herein are methods for preparing a lyophilized composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and lyophilizing the formulation to form a lyophilized composition.

Further provided herein are methods for preparing a spray-dried composition comprising obtaining a lipid carrier, wherein the lipid carrier is a nanoemulsion comprising a hydrophobic core, one or more inorganic nanoparticles and one or more lipids; incorporating one or more nucleic acid into the lipid carrier to form a lipid carrier-nucleic acid complex; adding at least one cryoprotectant to the lipid carrier-nucleic acid complex to form a formulation; and spray drying the formulation to form a spray-dried composition.

Further provided herein are methods for reconstituting a lyophilized composition comprising: obtaining a lipid carrier, wherein the lipid carrier is a nanoemulsion comprising a hydrophobic core, one or more inorganic nanoparticles, and one or more lipids; incorporating one or more nucleic acid into the said lipid carrier to form a lipid carrier-nucleic acid complex; adding at least one cryoprotectant to the lipid carrier-nucleic acid complex to form a formulation; lyophilizing the formulation to form a lyophilized composition; and reconstituting the lyophilized composition in a suitable diluent.

Further provided herein are methods for reconstituting a spray-dried composition comprising: obtaining a lipid carrier, wherein the lipid carrier is a nanoemulsion comprising a hydrophobic core, one or more inorganic nanoparticles, and one or more lipids, incorporating one or more nucleic acid into the said lipid carrier to form a lipid carrier-nucleic acid complex; adding at least one cryoprotectant to the lipid carrier-nucleic acid complex to form a formulation; spray drying the formulation to form a spray-dried composition; and reconstituting the spray-dried composition in a suitable diluent.

### Pharmaceutical Compostions

Provided herein is a lyophilized composition comprising a composition provided herein. Further provided herein is a suspension comprising a composition provided herein. In some embodiments, suspensions provided herein comprise a plurality of nanoparticles or compositions provided herein. In some embodiments, compositions provided herein are in a suspension, optionally a homogeneous suspension. In some embodiments, compositions provided herein are in an emulsion form.

Also provided herein is a pharmaceutical composition comprising a composition provided herein. In some embodiments, compositions provided herein are combined with pharmaceutically acceptable salts, excipients, and/or carriers to form a pharmaceutical composition. Pharmaceutical salts, excipients, and carriers may be chosen based on the route of administration, the location of the target issue, and the time course of delivery of the drug. A pharmaceutically acceptable carrier or excipient may include solvents, dispersion media, coatings, antibacterial and antifungal agents, isotonic and absorption delaying agents, etc., compatible with pharmaceutical administration.

In some embodiments, the pharmaceutical composition is in the form of a solid, semi-solid, liquid or gas (aerosol). Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Solid dosage forms for oral administration include capsules, tablets, pills, powders, and granules. In such solid dosage forms, the encapsulated or unencapsulated conjugate is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar-agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

### Dosing

Compositions provided herein may be formulated in dosage unit form for ease of administration and uniformity of dosage. A dosage unit form is a physically discrete unit of a composition provided herein appropriate for a subject to be treated. It will be understood, however, that the total usage of compositions provided herein will be decided by the attending physician within the scope of sound medical judgment. For any composition provided herein the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of compositions provided herein can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies may be used in formulating a range of dosage for human use.

### Administration

Provided herein are compositions and pharmaceutical compositions for administering to a subject in need thereof. In some embodiments, pharmaceutical compositions provided here are in a form which allows for compositions provided herein to be administered to a subject.

In some embodiments, the administering is local administration or systemic administration. In some embodiments, a composition described herein is formulated for administration/for use in administration via an intratumoral, subcutaneous, intradermal, intramuscular, inhalation, intravenous, intraperitoneal, intracranial, intranasal, intrathoracic, or intrathecal route. In some embodiments, the administering is every 1, 2, 4, 6, 8, 12, 24, 36, or 48 hours. In some embodiments, the administering is daily, weekly, or monthly. In some embodiments, the administering is repeated at least about every 28 days. In some embodiments, a composition or pharmaceutical composition provided herein is administered to the subject by two doses. In some embodiments, a second dose of a composition or pharmaceutical composition provided herein is administered about 28 days after the first dose. In some embodiments, a third dose of a composition or pharmaceutical composition provided herein is administered to a subject.

### Efficacy

Provided herein are nucleic acids that encode a protein, an antibody, or an antibody fragment, wherein upon administration to a cell, population of cells, or a subject the protein, the antibody, or the antibody fragment effectively neutralizes a non-enveloped virus. In some embodiments, the non-enveloped virus is a Picornaviridae virus. In some embodiments, the Picornaviridae virus is an enterovirus. Further provided herein are nucleic acids that encode for a protein, an antibody, or an antibody fragment that specifically binds to an EV-D68 viral protein. In some embodiments, the EV-D68 viral protein is a VP1 capsid protein.

Methods for assessing the presence of antibody neutralization of a virus or a viral antigen can be accomplished, e.g., by cellular impedance and live cell imaging assays. Cellular impedance assays include wells or plates with gold impedance biosensor arrays that measure the flow of electric current within a well that has been seeded with cells. Impedance is measured before, during, and after viral infection. During active viral infection, the interaction between the cells and the biosensors become weak and a small impedance of electric current (or increased flow of electric current) is detected as compared to cells that are not infected by a virus. Real-time impedance measurements can be used to track changes in cell number, cell size, cell-substrate attachment strength, and cell-cell interactions (i.e. barrier function). Because each of these parameters changes during a typical viral cytopathic effect (CPE), impedance provides a very sensitive readout of host cell health throughout the full continuum of a viral infection. Real-time impedance measurements in the presence and absence of a composition provided herein is useful to determine the effect of antibody function and suppression of CPE. Antibody-mediated suppression of the CPE is readily detected as changes in both the kinetics and magnitude of the impedance signal. Plotting the value of the impedance signal at various time points as a function of antibody concentration can produce a dose response curve to yield IC₅₀ measurements and determine the percentage of neutralization relative to control readings.

Provided herein are methods of modulating infectivity of a virus (e.g., an enterovirus or a coxsackievirus). In some embodiments, the methods comprise: contacting a cell or a population of cells with a virus or a viral antigen; contacting the cell or the population of cells with a composition provided herein; and identifying the presence or absence of one or more of: (1) viral neutralization; (2) antibody production; (3) viral plaques; and/or (4) cellular impedance relative to a comparable cell or a population of cells that have not been contacted with the composition provided herein. In some embodiments, the methods comprise: contacting a cell or a population of cells with a virus or a viral antigen; contacting the cell or the population of cells with a composition provided herein; and measuring one or more of: (1) viral neutralization; (2) antibody production; (3) viral plaques; and/or (4) cellular impedance relative to a comparable cell or a population of cells that have not been contacted with the composition provided herein. In some embodiments, the compositions provided herein increase viral neutralization; increase antibody production, reduce viral plaques, and/or increase cellular impedance relative to a comparable cell or a population of cells that have not been contacted with the composition provided herein. In some embodiments, the identifying or the measuring of (1) viral neutralization; (2) antibody production; (3) viral plaques; and/or (4) cellular impedance comprises a real time cellular impedance assay and/or live cell imaging assays.

### Therapeutic Applications

Provided herein are methods of treating a disease in a subject. In some embodiments, compositions described herein are used for the treatment of an infection. In some embodiments, the infection is a viral infection. In some embodiments, the viral infection is from an enterovirus. In some embodiments, the enterovirus is EV-D68.

In some embodiments, compositions described herein are used for the reduction of severity of an infection in a subject. In some embodiments, compositions described herein provide for reduction of severity or duration of symptoms associated with an infection in a subject. In some embodiments, the subject is at risk of developing a viral infection. In some embodiments, the subject does not display symptoms associated with active enterovirus infection. In some embodiments, the infection is a viral infection. In some embodiments, the viral infection is from an enterovirus. In some embodiments, the enterovirus is EV-D68.

### Kits

In some embodiments, a formulation of a composition described herein is prepared in a single container for administration. In some embodiments, a formulation of a composition described herein is prepared in two containers for administration, separating the nucleic acid and/or the compound provided herein from the nanoparticle carrier.

As used herein, “container” includes vessel, vial, ampule, tube, cup, box, bottle, flask, jar, dish, well of a single-well or multi-well apparatus, reservoir, tank, or the like, or other device in which the herein disclosed compositions may be placed, stored and/or transported, and accessed to remove the contents. Examples of such containers include glass and/or plastic sealed or re-sealable tubes and ampules, including those having a rubber septum or other sealing means that is compatible with withdrawal of the contents using a needle and syringe. In some implementations, the containers are RNase free.

Provided herein is kit, wherein the kit comprises: a first container comprising: a lipid carrier, wherein the lipid carrier comprises a hydrophobic core; and a kinase inhibitor; and a second container comprising: a nucleic acid encoding for a protein or a functional fragment thereof.

In some embodiments, the lipid carrier comprises a cationic lipid, an oil, and optionally an inorganic particle. In some embodiments, the inorganic particle comprises a metal. In some embodiments, the metal comprises metal salts, metal oxides, metal hydroxides, or metal phosphates. In some embodiments, the metal oxide comprises aluminum oxide, aluminum oxyhydroxide, iron oxide, titanium dioxide, or silicon dioxide. In some embodiments, the nucleic acid further codes for a RNA polymerase. In some embodiments, the RNA polymerase is a Venezuelan equine encephalitis virus (VEEV) RNA polymerase. In some embodiments, the nucleic acid sequence encoding for the RNA polymerase comprises the sequence of SEQ ID NO: 8. In some embodiments, the first container is lyophilized.

### EXEMPLARY EMBODIMENTS

Provided herein are compositions, wherein the compositions comprise: a nucleic acid sequence encoding for: a non-enveloped virus binding protein, wherein the non-enveloped virus binding protein comprises a heavy chain variable (V_{H}) region, wherein the non-enveloped virus binding protein specifically binds a structural protein of a non-enveloped virus; and an RNA-dependent RNA polymerase. Further provided herein are compositions, wherein the structural protein is a capsid protein. Further provided herein are compositions, wherein the capsid protein is a VP1 protein, a VP2 protein, a VP3 protein, or a VP4 protein. Further provided herein are compositions, wherein the structural protein is derived from a virus from the family Picornaviridae. Further provided herein are compositions, wherein the capsid protein is derived from an enterovirus, a coxsackievirus, a rhinovirus, a poliovirus, an echovirus, or a parechovirus. Further provided herein are compositions, wherein the capsid protein is derived from an enterovirus. Further provided herein are compositions, wherein the enterovirus is an enterovirus D68 (EV-D68). Further provided herein are compositions, wherein the nucleic acid is an RNA or a DNA. Further provided herein are compositions, wherein the nucleic acid encodes double-stranded RNA. Further provided herein are compositions, wherein the nucleic acid encodes single-stranded RNA. Further provided herein are compositions, wherein the RNA-dependent RNA polymerase includes a sub-genome of an alphavirus. Further provided herein are compositions, wherein the RNA-dependent RNA polymerase comprises a Venezuelan equine encephalitis virus (VEEV) RNA polymerase. Further provided herein are compositions, wherein the nucleic acid comprises an RNA sequence of SEQ ID NO: 8. Further provided herein are compositions, wherein the nucleic acid comprises an RNA sequence of SEQ ID NO: 8 and one of SEQ ID NO: 14 or SEQ ID NO: 15. Further provided herein are compositions, wherein the compositions further comprise a nanoparticle carrier. Further provided herein are compositions, wherein the nanoparticle carrier is a lipid nanoparticle carrier. Further provided herein are compositions, wherein the lipid nanoparticle carrier comprises a cationic lipid and a hydrophobic core. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate, ALC-0315, ((4-hydroxybutyl)azanediyl)bis(hexane-6,1-diyl)bis(2-hexyldecanoate), ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate, BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide, cKK-E12, 3,6-bis(4-(bis(2-hydroxydodecyl)amino)butyl)piperazine-2,5-dione, DC-Cholesterol, 3β-[N—(N′,N′-dimethylaminoethane)-carbamoyl]cholesterol, DLin-MC3-DMA, (6Z,9Z,28Z,31Z)-heptatriaconta-6,9,28,31-tetraen-19-yl 4-(dimethylamino) butanoate, DOPE, 1,2-dioleoyl-sn-glycero-3-phosphoethanolamine, DOSPA, 2,3-dioleyloxy-N-[2-(sperminecarboxamido)ethyl]-N,N-dimethyl-1-propanaminium trifluoroacetate, DSPC, 1,2-distearoyl-sn-glycero-3-phosphocholine, ePC, ethylphosphatidylcholine, FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate, Lipid H (SM-102), heptadecan-9-yl 8-((2-hydroxyethyl)(6-oxo-6-(undecyloxy)hexyl)amino) octanoate, OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate), PEG2000-DMG, (R)-2,3-bis(myristoyloxy)propyl-1-(methoxy poly(ethylene glycol)2000) carbamate, TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil comprises a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle carrier further comprises an inorganic particle. Further provided herein are compositions, wherein the inorganic particle is in a solid phase. Further provided herein are compositions, wherein the inorganic particle is coated with a capping ligand and a surfactant. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle carrier comprises a cationic lipid and an oil. Further provided herein are compositions, wherein the nanoparticle carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the hydrophobic core further comprises: a phosphate-terminated lipid; and a surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA). Further provided herein are compositions, wherein the nucleic acid is present in an amount of 5 micrograms (μg) to about 200 μg. Further provided herein are compositions, wherein the nucleic acid is present in an amount of up to about 25 nanograms (ng), about 50 ng, about 75 ng, about 100 ng, about 150 ng, or about 175 ng. Further provided herein are compositions, wherein the nucleic acid is present in an amount of up to about 1 μg. Further provided herein are compositions, wherein the nucleic acid is present in an amount of about 0.05 micrograms (μg), about 0.1 μg, about 0.2 μg, about 0.5 μg, about 1 μg, about 5 μg, about 10 μg, about 12.5 μg, about 15 μg, about 25 μg, about 40 μg, about 50 μg, about 100 μg, about 150 μg, or about 200 μg. Further provided herein are compositions, wherein the composition is lyophilized. Further provided herein are compositions, wherein the composition is in a liquid, semi-liquid, solution, propellant, or powder dosage form. Further provided herein are compositions, wherein the composition is formulated as a suspension. Further provided herein are compositions, wherein the suspension is a homogeneous suspension. Further provided herein are compositions, wherein the lipid nanoparticle carrier is in an aqueous solution.

Provided herein are compositions, wherein the compositions comprise: an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of SEQ ID NOS: 1-7. Further provided herein are compositions, wherein the V_{H} region comprises a sequence having at least 90% sequence identity to any one of SEQ ID NOS: 2-4. Further provided herein are compositions, wherein the V_{H} region comprises a sequence having at least 90% sequence identity to any one of SEQ ID NOS: 5-7. Further provided herein are compositions, wherein the V_{H} region comprises a sequence having at least 95% sequence identity to any one of SEQ ID NOS: 1-7. Further provided herein are compositions, wherein the V_{H} region comprises a sequence having at least 99% sequence identity to any one of SEQ ID NOS: 1-7. Further provided herein are compositions, wherein the V_{H} region comprises any one of SEQ ID NOS: 1-7 or a functional fragment thereof. Further provided herein are compositions, wherein the binding protein is an antigen-binding fragment, optionally wherein the antigen binding fragment is fused to a glycosylphosphatidylinositol anchor, an Fc domain, or a combination glycosylphosphatidylinositol-Fc fusion. Further provided herein are compositions, wherein the antigen-binding fragment is a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the antigen binding fragment is a nanobody. Further provided herein are compositions, wherein the EV-D68 belongs to clade A, B1, B2, B3, C, or D. Further provided herein are compositions, wherein the EV-D68 is US/MO/14-18947-EV-D68. Further provided herein are compositions, wherein the compositions comprise a nucleic acid encoding for an RNA-dependent RNA polymerase.

Provided herein are compositions, wherein the compositions comprise: a nucleic acid encoding for an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of SEQ ID NOS: 1-7. Further provided herein are compositions, wherein the V_{H} region comprises a sequence having at least 90% sequence identity to any one of SEQ ID NOS: 2-4. Further provided herein are compositions, wherein the V_{H} region comprises a sequence having at least 90% sequence identity to any one of SEQ ID NOS: 5-7. Further provided herein are compositions, wherein the V_{H} region comprises a sequence having at least 95% sequence identity to any one of SEQ ID NOS: 1-7. Further provided herein are compositions, wherein the V_{H} region comprises a sequence having at least 99% sequence identity to any one of SEQ ID NOS: 1-7. Further provided herein are compositions, wherein the V_{H} region comprises any one of SEQ ID NOS: 1-7 or a functional fragment thereof. Further provided herein are compositions, wherein the nucleic acid further comprises a region encoding for an RNA-dependent RNA polymerase. Further provided herein are compositions, wherein the compositions further comprise a nanoparticle carrier. Further provided herein are compositions, wherein the nanoparticle carrier is a lipid nanoparticle carrier. Further provided herein are compositions, wherein the nanoparticle carrier comprises a hydrophobic core. Further provided herein are compositions, wherein the hydrophobic core comprises a liquid organic material. Further provided herein are compositions, wherein the hydrophobic core comprises a solid inorganic material. Further provided herein are compositions, wherein the nanoparticle carrier comprises a hydrophilic surface. Further provided herein are compositions, wherein the nanoparticle carrier is up to 120 nm in diameter. Further provided herein are compositions, wherein the nanoparticle carrier is 40 to 80 nm in diameter. Further provided herein are compositions, wherein the nanoparticle carrier is 50 to 70 nm in diameter. Further provided herein are compositions, wherein the nanoparticle carrier comprises a membrane. Further provided herein are compositions, wherein the nanoparticle carrier comprises a cationic lipid. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil is a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle carrier comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle carrier comprises a cationic lipid, an oil, and an inorganic particle. Further provided herein are compositions, wherein the nanoparticle carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the nanoparticle carrier comprises a cationic lipid, an oil, and a surfactant. Further provided herein are compositions, wherein the nanoparticle carrier comprises a cationic lipid, an oil, an inorganic particle, and a surfactant. Further provided herein are compositions, wherein the hydrophobic core comprises: a phosphate-terminated lipid; a surfactant; and optionally one or more inorganic particles. Further provided herein are compositions, wherein the hydrophobic core comprises: one or more inorganic particles; a phosphate-terminated lipid; and a surfactant. Further provided herein are compositions, wherein each inorganic particle is coated with a capping ligand or the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA), oleic acid, oleylamine or sodium dodecyl sulfate (SDS). Further provided herein are compositions, wherein the nanoparticle carrier is dispersed in an aqueous solution. Further provided herein are compositions, wherein the nucleic acid is an RNA or a DNA. Further provided herein are compositions, wherein the RNA polymerase is a Venezuelan equine encephalitis virus (VEEV) RNA polymerase. Further provided herein are compositions, wherein the nucleic acid encoding the RNA-dependent RNA polymerase comprises a nucleic acid sequence that is at least 90% identical to SEQ ID NO: 8. Further provided herein are compositions, wherein the nucleic acid encoding the RNA-dependent RNA polymerase comprises SEQ ID NO: 8. Further provided herein are compositions, wherein the V_{H} region has an amino acid sequence that is at least 95% sequence identity to any one of SEQ ID NOS: 1-7. Further provided herein are compositions, wherein the V_{H} region comprises an amino acid sequences that has at least 95% identity to any one of SEQ ID NOS: 2-7. Further provided herein are compositions, wherein the V_{H} region has an amino acid sequence that is at least 99% sequence identity to any one of SEQ ID NOS: 1-7. Further provided herein are compositions, wherein the V_{H} region comprises an amino acid sequences that has at least 99% identity to any one of SEQ ID NOS: 2-7. Further provided herein are compositions, wherein the V_{H} region has an amino acid sequence comprising any one of SEQ ID NOS: 1-7. Further provided herein are compositions, wherein the V_{H} region comprises an amino acid sequences comprising any one of SEQ ID NOS: 2-7. Further provided herein are compositions, wherein the nucleic acid comprises a sequence that is at least 90% identical to SEQ ID NO: 12 or SEQ ID NO: 13. Further provided herein are compositions, wherein the nucleic acid comprises a sequence that is at least 95% identical to SEQ ID NO: 12 or SEQ ID NO: 13. Further provided herein are compositions, wherein the nucleic acid comprises a sequence that is at least 99% identical to SEQ ID NO: 12 or SEQ ID NO: 13. Further provided herein are compositions, wherein the nucleic acid comprises a nucleic acid sequence of SEQ ID NO: 12 or SEQ ID NO: 13. Further provided herein are compositions, wherein the composition is lyophilized.

Provided herein are compositions, wherein the compositions comprise: a nanoparticle carrier; and a nucleic acid, wherein the nucleic acid comprises: (i) a region encoding for an RNA-dependent RNA polymerase; (ii) a region encoding for a non-enveloped virus structural protein; and (iii) a region encoding for a virus protease, wherein the virus structural protein is a substrate for the virus protease. Further provided herein are compositions, wherein the nucleic acid is an RNA. Further provided herein are compositions, wherein the virus protease is 3CD. Further provided herein are compositions, wherein the nucleic acid comprises open reading frames for both (ii) the region encoding the virus structural protein and (iii) the region encoding the virus protease. Further provided herein are compositions, wherein the nanoparticle carrier comprises a hydrophobic core. Further provided herein are compositions, wherein the hydrophobic core comprises a liquid organic material. Further provided herein are compositions, wherein the hydrophobic core comprises a solid inorganic material. Further provided herein are compositions, wherein the nanoparticle carrier comprises a hydrophilic surface. Further provided herein are compositions, wherein the nanoparticle carrier is up to 120 nm in diameter. Further provided herein are compositions, wherein the nanoparticle carrier is 40 to 80 nm in diameter. Further provided herein are compositions, wherein the nanoparticle carrier is 50 to 70 nm in diameter. Further provided herein are compositions, wherein the nanoparticle carrier is dispersed in an aqueous solution. Further provided herein are compositions, wherein the nanoparticle carrier comprises a membrane. Further provided herein are compositions, wherein the nanoparticle carrier comprises a cationic lipid. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FT5, hexa(octan-3-yl) 9,9′,9″,9″″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil is a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle carrier further comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle carrier comprises a cationic lipid, an oil, and optionally an inorganic particle. Further provided herein are compositions, wherein the nanoparticle carrier comprises a cationic lipid, an oil, and an inorganic particle. Further provided herein are compositions, wherein the nanoparticle carrier further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the nanoparticle carrier comprises a cationic lipid, an oil, and a surfactant. Further provided herein are compositions, wherein the nanoparticle carrier comprises a cationic lipid, an oil, an inorganic particle, and a surfactant. Further provided herein are compositions, wherein the hydrophobic core comprises: a phosphate-terminated lipid; a surfactant; and optionally one or more inorganic particles. Further provided herein are compositions, wherein the hydrophobic core comprises: one or more inorganic particles; a phosphate-terminated lipid; and a surfactant. Further provided herein are compositions, wherein each inorganic particle is coated with a capping ligand or the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA), oleic acid, oleylamine or sodium dodecyl sulfate (SDS).

Provided herein are compositions, wherein the compositions comprise: an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of the sequences listed in Table 1 (SEQ ID NOS: 1-4) or Table 2 (SEQ ID NOS: 5-7). Further provided herein are compositions, wherein the V_{H} region comprises a sequence having at least 95% sequence identity to any one of the sequences listed in Table 1 (SEQ ID NOS: 1-4), optionally a sequence having at least 95% sequence identity to any comprises any one of SEQ ID NOS: 2-4. Further provided herein are compositions, wherein the V_{H} region wherein the V_{H} region comprises a sequence having at least 90% sequence identity to any one of SEQ ID NOS: 5-7. Further provided herein are compositions, wherein the binding protein is an antigen-binding fragment, optionally wherein the antigen binding fragment is fused to a glycosylphosphatidylinositol anchor, an Fc domain, or a combination glycosylphosphatidylinositol-Fc fusion. Further provided herein are compositions, wherein the antigen-binding fragment is a single domain antibody, a diabody, a scFv, an scFv dimer, a BsFv, a dsFv, a (dsFv)2, a dsFv-dsFv′, an Fv fragment, a Fab, a Fab′, a F(ab′)2, a ds-diabody, a nanobody, a domain antibody, or a bivalent domain antibody. Further provided herein are compositions, wherein the antigen binding fragment is a nanobody. Further provided herein are compositions, wherein the EV-D68 belongs to clade A, B1, B2, B3, C, or D. Further provided herein are compositions, wherein the EV-D68 is US/MO/14-18947-EV-D68.

Provided herein are compositions, wherein the compositions comprise: a nucleic acid encoding for an enterovirus D68 (EV-D68) binding protein comprising a heavy chain variable (V_{H}) region, wherein the EV-D68 binding protein specifically binds an EV-D68 epitope comprising an EV-D68 P1 capsid protein, and wherein the V_{H} region comprises an amino acid sequence having at least 90% sequence identity to of any one of the sequences listed in Table 1 (SEQ ID NOS: 1-4) or Table 2 (SEQ ID NOS: 5-7). Further provided herein are compositions comprising a nanoparticle. Further provided herein are compositions, wherein the nanoparticle comprises a hydrophobic core. Further provided herein are compositions, wherein the hydrophobic core comprises a liquid organic material. Further provided herein are compositions, wherein the hydrophobic core comprises a solid inorganic material. Further provided herein are compositions, wherein the nanoparticle comprises a hydrophilic surface. Further provided herein are compositions, wherein the nanoparticle is up to 120 nm in diameter. Further provided herein are compositions, wherein the nanoparticle is 40 to 80 nm in diameter. Further provided herein are compositions, wherein the nanoparticle is 50 to 70 nm in diameter. Further provided herein are compositions, wherein the nanoparticle is dispersed in an aqueous solution. Further provided herein are compositions, wherein the nanoparticle comprises a membrane. Further provided herein are compositions, wherein the nanoparticle comprises a cationic lipid. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FITS, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil is a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle comprises a cationic lipid, an oil, and an inorganic particle. Further provided herein are compositions, wherein the nanoparticle further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the nanoparticle comprises a cationic lipid, an oil, an inorganic particle, and a surfactant. Further provided herein are compositions, wherein the hydrophobic core comprises: one or more inorganic particles; a phosphate-terminated lipid; and a surfactant. Further provided herein are compositions, wherein each inorganic particle is coated with a capping ligand or the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA), oleic acid, oleylamine or sodium dodecyl sulfate (SDS). Further provided herein are compositions, wherein the nucleic acid is an RNA or a DNA. Further provided herein are compositions, wherein the nucleic acid further codes for an RNA polymerase. Further provided herein are compositions, wherein the RNA polymerase is a Venezuelan equine encephalitis virus (VEEV) RNA polymerase. Further provided herein are compositions, wherein the nucleic acid coding the RNA polymerase comprises the nucleic acid sequence of SEQ ID NO: 8. Further provided herein are compositions, wherein the V_{H} region has at least 95% sequence identity to any one of the sequences listed in Table 1 (SEQ ID NOS: 1-4) or Table 2 (SEQ ID NOS: 5-7). Further provided herein are compositions, wherein the V_{H} region comprises any one of SEQ ID NOS: 2-7. Further provided herein are compositions, wherein the nucleic acid comprises the nucleic acid sequence of SEQ ID NO: 8 or 9. Further provided herein are compositions, wherein the composition is lyophilized. Further provided herein are compositions, wherein the nanoparticle comprises any one of NP-1 to NP-37.

Provided herein are compositions, wherein the compositions comprise: a nanoparticle; and a nucleic acid, wherein the nucleic acid comprises: a region encoding for an RNA polymerase; a region encoding for a virus structural protein, wherein the virus is a non-enveloped virus; and a region encoding for a virus protease, wherein the virus structural protein is a substrate for the virus protease. Further provided herein are compositions, wherein nucleic acid is an RNA. Further provided herein are compositions, wherein the virus protease is 3CD. Further provided herein are compositions, wherein the nucleic acid comprises open reading frames for both (ii) the region encoding the virus structural protein and (iii) the region encoding the virus protease. Further provided herein are compositions, wherein the nanoparticle comprises a hydrophobic core. Further provided herein are compositions, wherein the hydrophobic core comprises a liquid organic material. Further provided herein are compositions, wherein the hydrophobic core comprises a solid inorganic material. Further provided herein are compositions, wherein the nanoparticle comprises a hydrophilic surface. Further provided herein are compositions, wherein the nanoparticle is up to 120 nm in diameter. Further provided herein are compositions, wherein the nanoparticle is 40 to 80 nm in diameter. Further provided herein are compositions, wherein the nanoparticle is 50 to 70 nm in diameter. Further provided herein are compositions, wherein the nanoparticle is dispersed in an aqueous solution. Further provided herein are compositions, wherein the nanoparticle comprises a membrane. Further provided herein are compositions, wherein the nanoparticle comprises a cationic lipid. Further provided herein are compositions, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3′″-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; 0-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″Z,9″Z,12Z,12′Z,12″Z,12″Z)-tetrakis (octadeca-9,12-dienoate); PEG2000-DMG, (R)-2,3-bis(myristoyloxy)propyl-1-(methoxy poly(ethylene glycol)2000) carbamate; TT3, or N1,N3,N5-tris(3-(didodecylamino)propyl)benzene-1,3,5-tricarboxamide. Further provided herein are compositions, wherein the hydrophobic core comprises an oil. Further provided herein are compositions, wherein the oil is in liquid phase. Further provided herein are compositions, wherein the oil is a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E. Further provided herein are compositions, wherein the triglyceride is capric triglyceride, caprylic triglyceride, a caprylic and capric triglyceride, a triglyceride ester, or myristic acid triglycerin. Further provided herein are compositions, wherein the nanoparticle comprises an inorganic particle. Further provided herein are compositions, wherein the inorganic particle is within the hydrophobic core. Further provided herein are compositions, wherein the inorganic particle comprises a metal. Further provided herein are compositions, wherein the metal comprises a metal salt, a metal oxide, a metal hydroxide, or a metal phosphate. Further provided herein are compositions, wherein the metal oxide comprises aluminum oxide, aluminum oxyhydroxide, iron oxide, titanium dioxide, or silicon dioxide. Further provided herein are compositions, wherein the nanoparticle comprises a cationic lipid, an oil, and an inorganic particle. Further provided herein are compositions, wherein the nanoparticle further comprises a surfactant. Further provided herein are compositions, wherein the surfactant is a hydrophobic surfactant. Further provided herein are compositions, wherein the hydrophobic surfactant is sorbitan monolaurate, sorbitan monopalmitate, sorbitan monostearate, sorbitan monooleate, or sorbitan trioleate. Further provided herein are compositions, wherein the surfactant is a hydrophilic surfactant. Further provided herein are compositions, wherein the hydrophilic surfactant is a polysorbate. Further provided herein are compositions, wherein the nanoparticle comprises a cationic lipid, an oil, an inorganic particle, and a surfactant. Further provided herein are compositions, wherein the hydrophobic core comprises: one or more inorganic particles; a phosphate-terminated lipid; and a surfactant. Further provided herein are compositions, wherein each inorganic particle is coated with a capping ligand or the surfactant. Further provided herein are compositions, wherein the phosphate-terminated lipid is trioctylphosphine oxide (TOPO). Further provided herein are compositions, wherein the surfactant is a phosphorous-terminated surfactant, a carboxylate-terminated surfactant, a sulfate-terminated surfactant, or an amine-terminated surfactant. Further provided herein are compositions, wherein the surfactant is distearyl phosphatidic acid (DSPA), oleic acid, oleylamine or sodium dodecyl sulfate (SDS). Further provided herein are compositions, wherein the nucleic acid is an RNA or a DNA. Further provided herein are compositions, wherein the nucleic acid further codes for an RNA polymerase. Further provided herein are compositions, wherein the RNA polymerase is a Venezuelan equine encephalitis virus (VEEV) RNA polymerase. Further provided herein are compositions, wherein the nucleic acid coding the RNA polymerase comprises the nucleic acid sequence of SEQ ID NO: 8. Further provided herein are compositions, wherein the V_{H} region has at least 95% sequence identity to any one of the sequences listed in Table 1 (SEQ ID NOS: 1-4) or Table 2 (SEQ ID NOS: 5-7). Further provided herein are compositions, wherein the V_{H} region comprises any one of SEQ ID NOS: 2-7. Further provided herein are compositions, wherein the nucleic acid comprises the nucleic acid sequence of SEQ ID NO: 8 or 9. Further provided herein are compositions, wherein the composition is lyophilized. Further provided herein are compositions, wherein the nanoparticle comprises any one of NP-1 to NP-37.

Provided herein are suspensions, wherein the suspensions comprise a composition provided herein.

Provided herein are pharmaceutical compositions, wherein the pharmaceutical compositions comprise a composition provided herein; and a pharmaceutical excipient.

Provided herein are methods for treatment of an infection in a subject, the method comprising: administering to a subject, the composition provided herein, the suspension provided herein, or the pharmaceutical composition provided herein, thereby treating the infection in the subject. Further provided herein are methods, wherein the administering is systemic. Further provided herein are methods, wherein the administering is intranasal, subcutaneous, intravenous, via inhalation, intratracheal, or intramuscular. Further provided herein are methods, wherein the subject does not display symptoms associated with active enterovirus infection. Further provided herein are methods, wherein the subject has symptoms associated with active enterovirus infection. Further provided herein are methods, wherein the treatment reduces severity of the infection. Further provided herein are methods, wherein the infection is an enterovirus infection, a coxsackievirus infection, a rhinovirus infection, a poliovirus infection, an echovirus infection, or a parechovirus infection.

Provided herein are methods for modulating an immune response in subject, the methods comprising: administering to a subject, the composition provided herein, the suspension provided herein, or the pharmaceutical composition provided herein, thereby modulating an immune in the subject. Further provided herein are methods, wherein the administering is intranasal, subcutaneous, intravenous, via inhalation, intratracheal, or intramuscular. Further provided herein are methods, wherein the subject has, is diagnosed with, or is at risk of developing a Picornaviridae infection. Further provided herein are methods, wherein the Picornaviridae infection is an enterovirus infection. Further provided herein are methods, wherein the enterovirus infection is and EV-D68 infection. Further provided herein are methods, wherein the EV-D68 infection is caused by an EV-D68 virus that belongs to clade A, B1, B2, B3, C, or D. Further provided herein are methods, wherein the EV-D68 virus is US/MO/14-18947-EV-D68. Further provided herein are methods, wherein the administering reduces the risk of an enterovirus infection by at least 10% relative to a subject that has not been administered the composition, the suspension, or the pharmaceutical composition.

Provided herein are methods for the treatment of an infection in a subject, the methods comprising: administering to a subject, the composition provided herein, the suspension provided herein, or the pharmaceutical composition provided herein, thereby treating the infection in the subject.

Provided herein are methods for treatment of enterovirus infection, the methods comprising administering to a subject the enterovirus D68 (EV-D68) binding protein as described herein. Further provided herein are methods, wherein the administering is intranasal, subcutaneous, intravenous, via inhalation, or intramuscular. Further provided herein are methods, wherein the subject does not display symptoms associated with active enterovirus infection. Further provided herein are methods, wherein the administering is systemic. Further provided herein are methods, wherein the treatment reduces severity of the enterovirus infection.

Further provided herein are methods for treatment of enterovirus infection, the methods comprising administering to a subject: comprising administering to a subject, the nucleic acid as described herein. Further provided herein are methods, wherein the administering is intranasal, subcutaneous, intravenous, via inhalation, or intramuscular. Further provided herein are methods, wherein the subject does not display symptoms associated with active enterovirus infection. Further provided herein are methods, wherein the administering is systemic. Further provided herein are methods, wherein the treatment reduces severity of the enterovirus infection.

Provided herein are methods for antibody generation, comprising: administering to a mammal a composition, wherein the composition supports formation of a non-enveloped viral protein in the mammal and comprises: a carrier; and a nucleic acid, wherein the nucleic acid comprises: a region encoding for an RNA polymerase; a region encoding for a virus structural protein, wherein the virus is a non-enveloped virus; and a region encoding for a virus protease, wherein the virus structural protein is a substrate for the viral protease.

### EXAMPLES

### Example 1: Manufacture and Stability of Nanoparticles—NP-1, NP-3, and NP-30

i. Manufacture of NP-1. NP-1 particles comprise 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID), 0.2 mg Fe/ml 12 nm oleic acid-coated iron oxide nanoparticles (ImagionBio) and 10 mM sodium citrate dihydrate (Fisher Chemical). 1 ml of 20 mg Fe/ml 12 nm diameter oleic acid-coated iron oxide nanoparticles in chloroform (ImagionBio, lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 pm diamond interaction chamber and an auxiliary H30Z-200 pm ceramic interaction chamber until the z-average hydrodynamic diameter—measured by dynamic light scattering (Malvern Zetasizer Nano S)—reached 40-80 nm with a 0.1-0.25 polydispersity index (PDI). The microfluidized NP-1 was terminally filtered with a 200 nm pore-size polyethersulfone (PES) filter and stored at 2-8 degrees Celsius (° C.). Iron concentration was determined by ICP-OES. DOTAP and Squalene concentration were measured by RP-HPLC.

ii. Manufacture of NP-3. NP-3 particles comprise 37.5 mg/ml Miglyol 812 N (IOI Oleo GmbH), 37 mg/ml Span® 60 (Millipore Sigma), 37 mg/ml Tween® 80 (Fisher Chemical), 30 mg/ml DOTAP chloride (LIPOID), 0.2 mg Fe/ml 15 nm oleic acid-coated iron oxide nanoparticles (ImagionBio) and 10 mM sodium citrate dihydrate (Fisher Chemical). 1 ml of 20 mg Fe/ml 15 nm diameter oleic acid-coated iron oxide nanoparticles in chloroform (ImagionBio, Lot #95-127) were washed three times by magnetically separating in a 4:1 acetone:chloroform (v/v) solvent mixture. After the third wash, the volatile solvents (acetone and chloroform) were allowed to completely evaporate in a fume hood leaving behind a coating of dried oleic acid iron oxide nanoparticles. To this iron oxide coating, 3.75 grams squalene, 3.7 grams span 60, and 3 grams DOTAP were added to produce the oil phase. The oil phase was sonicated for 45 minutes in a 65° C. water bath. Separately, the aqueous phase was prepared by dissolving 19.5 grams Tween 80 in 500 ml of 10 mM sodium citrate buffer prepared in nuclease free water. 92 ml of the aqueous phase was transferred to a separate glass bottle and heated to 65° C. for 30 minutes. The oil phase was mixed with the 92 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 pm diamond interaction chamber and an auxiliary H30Z-200 pm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized NP-3 was terminally filtered with a 200 nm pore-size polyethersulfone (PES) filter and stored at 2-8° C. Iron concentration was determined by ICP-OES. DOTAP concentration was measured by RP-HPLC.

iii. Manufacture of NP-30. A lipid carrier without providing inorganic core particles in the core was generated having 37.5 mg/ml squalene (SEPPIC), 37 mg/ml Span® 60 (Millipore Sigma), 37 mg/ml Tween® 80 (Fisher Chemical), 30 mg/ml DOTAP chloride (LIPOID) and 10 mM sodium citrate. To a 200 ml beaker 3.75 grams squalene, 3.7 grams span 60, and 3.0 grams DOTAP were added to produce the oil phase. The oil phase was sonicated for 45 minutes in a 65 degrees Celsius water bath. Separately, the aqueous phase was prepared by dissolving 19.5 grams Tween 80 in 500 ml of 10 mM sodium citrate buffer prepared in nuclease free water. 96 ml of the aqueous phase was transferred to a separate glass bottle and heated to 65 degrees Celsius for 30 minutes. The oil phase was mixed with the 96 ml of aqueous phase by adding the warm oil phase to the warm aqueous phase. The mixture was emulsified using a VWR 200 homogenizer (VWR International) and the resulting crude emulsion was processed by passaging through a M110P microfluidizer (Microfluidics) at 30,000 psi equipped with a F12Y 75 pm diamond interaction chamber and an auxiliary H30Z-200 pm ceramic interaction chamber until the z-average hydrodynamic diameter—measured by dynamic light scattering (Malvern Zetasizer Nano S)—reached 40-80 nm with a 0.1-0.3 polydispersity index (PDI). The microfluidized NP-30 without inorganic core formulation was terminally filtered with a 200 nm pore-size polyethersulfone (PES) filter and stored at 2-8 degrees Celsius (° C.). DOTAP and Squalene concentration were measured by RP-HPLC.

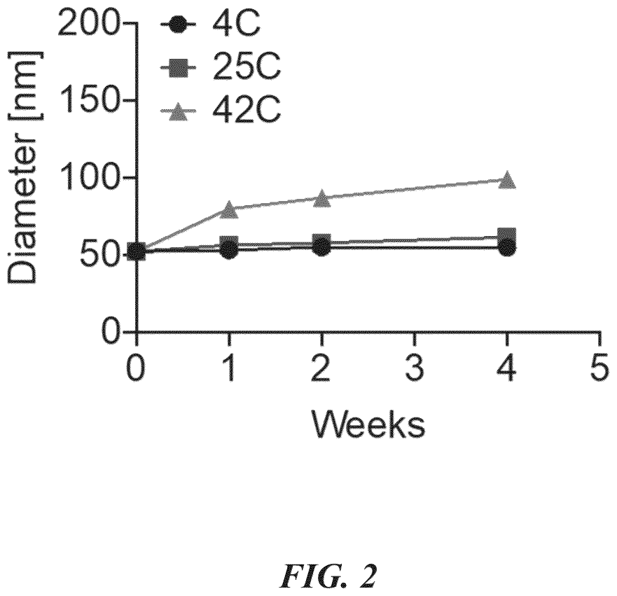

Stability. A nanoparticle according to NP-1 was placed into a stability chamber at the indicated temperatures. The stability was determined by particle size measurement using dynamic light scattering. The results show that the NP-1 formulation formed a stable colloid when stored at 4, 25 and 42 degrees Celsius. Time measurements were taken over 4 weeks. As shown in FIG. 2, the range of nanoparticle size was about 50-100 nm in diameter, and closer to 40-60 nm in diameter for the 4 and 25 degrees Celsius conditions over time.

### Example 2: Self-Replicating mRNA Construct

A plasmid encoding a T7 promoter followed by the 5′ and 3′ UTRs and nonstructural genes of Venezuelan equine encephalitis virus (VEEV) strain TC-83 was generated using standard DNA synthesis and cloning methods. The VEEV replicon mRNA backbone is set forth in SEQ ID NO: 8.

### Example 3: EV-D68 Antibodies as Protective Against Disease

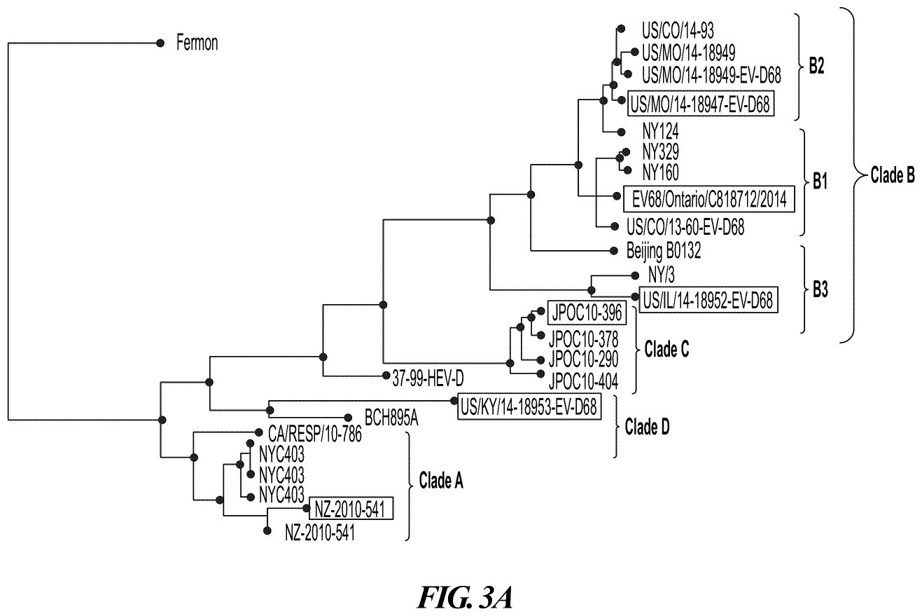

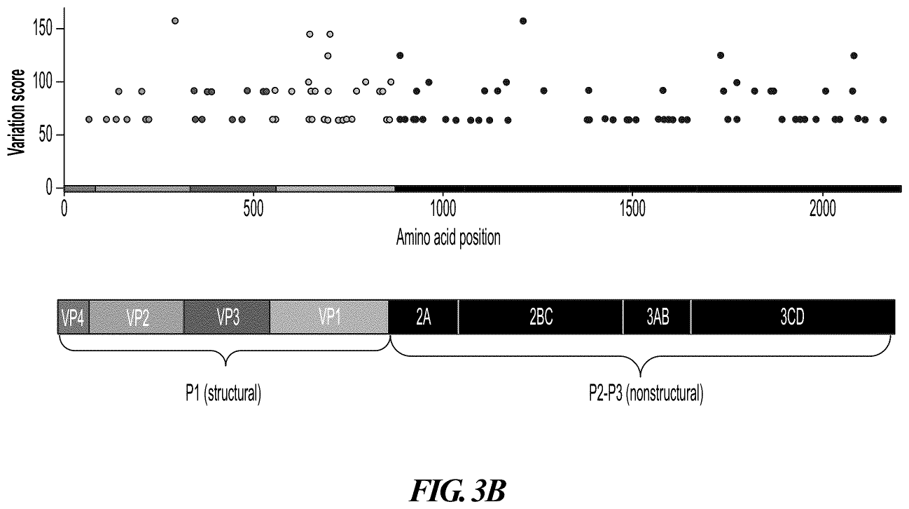

EV-D68 is a single serotype of the EV-D species within the Enterovirus genus. EV-D68 can be further divided into 4 clades (A, B, C, and D) and clade B further divided into 3 subclades (B1, B2, and B3) based on genotype. Full-genome sequences of contemporary EV-D68 isolates from recent outbreaks were aligned (MUSCLE) and a maximum-likelihood phylogenetic tree was constructed (PhyML) using a GTR best-fit nucleotide substitution model. A single isolate (red box) from each clade was then selected for protein sequence variation analysis (FIG. 3A). Like other enteroviruses, EV-D68 possesses a single-stranded positive-sense RNA genome encoding a single open reading frame that can be divided into the structural P1 and nonstructural P2-P3 polyproteins. Variation in protein sequence between the 6 isolates was scored at each amino acid position (ViPR) and plotted. FIG. 3B shows the color-coded genome organization of enteroviruses. The former polyprotein is further divided into the VP1, VP2, VP3, and VP4 capsid proteins while the latter includes the 3CD protease which initiates the cleavage of P1 into the 4 capsid monomers. While all subclades depicted in FIG. 3A fall into a single serotype, amino acid variation within the structural, antigenic proteins does exist, particularly in the VP1 subunit of capsid (FIG. 3B), a major target for neutralizing antibodies. Of the few epidemiological and pre-clinical studies on the development of vaccines and therapeutics against EV-D68 infection, the consensus is that antibodies likely play a major role in protection from disease. Evidence for this supposition includes the presence of EV-D68-neutralizing antibodies in many adult human sera as well as the ability of antibodies to protect against neuromuscular disease and death in murine models of EV-D68 infection.

### Example 4: Generation of Neutralizing Antibodies in Alpacas

Neutralizing antibodies against conformationally-native and diverse epitopes presented on EV-D68 VLPs launched from repRNA in vivo were induced in alpacas using repRNA/NP-1 immunization. To identify broadly reactive and cross-neutralizing antibodies, repRNA encoding VLPs from the 6 sub-clades of the EV-D68 serotype were designed and used. VLPs of enveloped viruses, including those of alphaviruses and flaviviruses that bud from the host cell, are produced in vivo following DNA/RNA genetic immunization approaches. However, in vivo production of VLPs derived from non-enveloped viruses has yet to be demonstrated and is complicated by the involvement of nonstructural viral proteases required for processing of the structural polyprotein in trans. It is established that co-expression of the P1 and 3CD proteins of enteroviruses in insect and mammalian cell lines results in efficient formation of VLPs for enterovirus 71, coxsackievirus A16, coxsackievirus A6, as well as EV-D68.

### Example 5: Gene Design

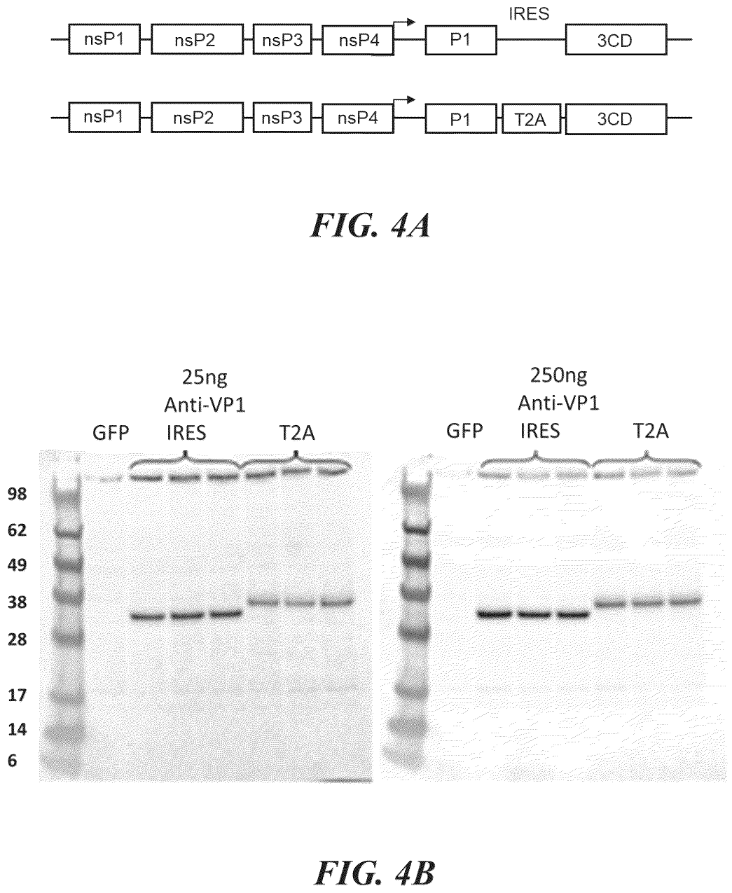

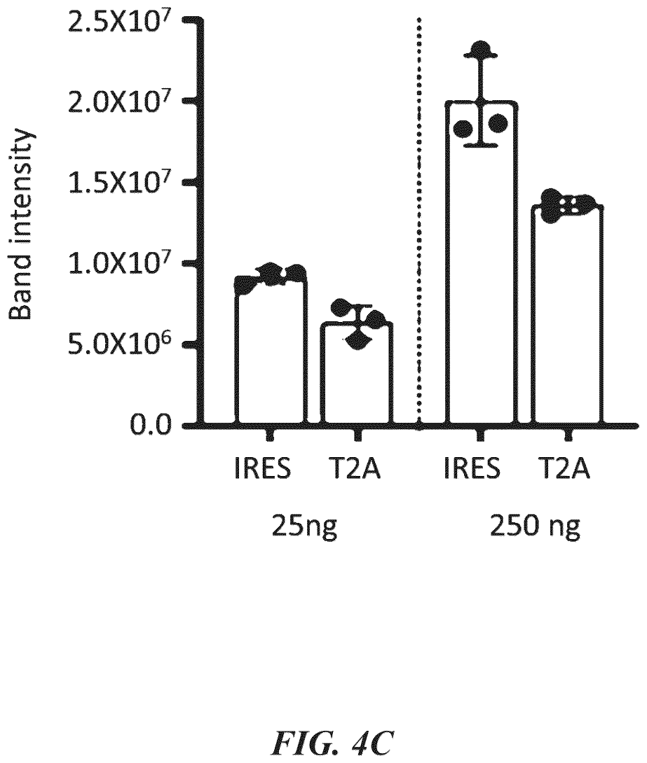

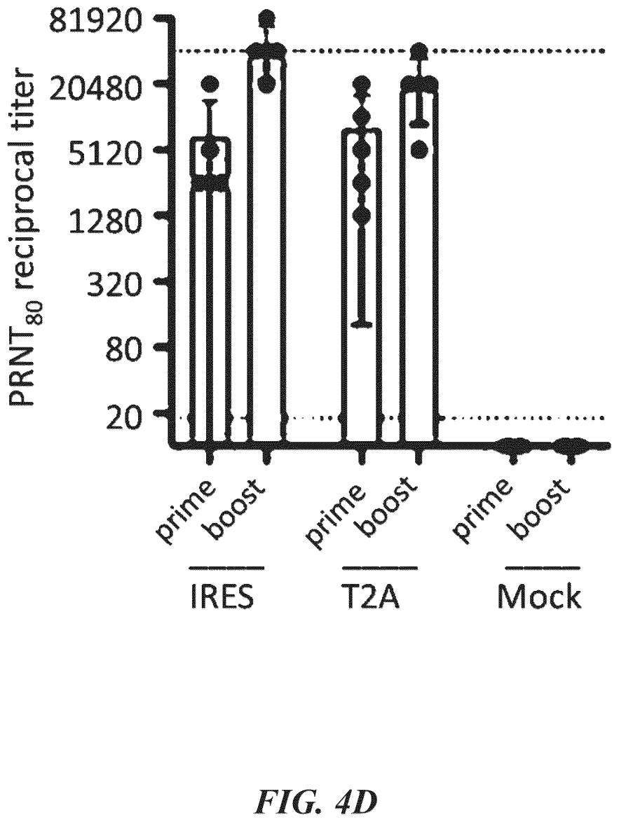

Following the model of virion production described in Example 4, repRNAs encoding the P1 and 3CD protein genes of EV-D68 (US/MO/14-18947) were designed, where both open reading frames were encoded on a single repRNA molecule separated by either an internal ribosomal entry site (IRES) or by a ribosomal skipping peptide sequence derived from thosea asigna virus (T2A) to facilitate production of both proteins from the same RNA molecule (FIG. 4A). RNA was then prepared by in vitro transcription and capping and protein production was evaluated by semi-quantitative western blot of BHK cells transfected with each RNA. Using an anti-VP1 antibody, production of the correctly processed protein (VP1 ˜34 kDa) in cell lysates was shown (FIG. 4B). A slight increase in the molecular weight of VP1 when using the T2A-mediated expression of 3CD was seen. This is most likely due to residual T2A amino acids fused to the C-terminus of VPL.

Densitometric analysis of the western blots was performed and it was concluded that the IRES-based approach resulted in more efficient VLP production (FIG. 4C). The immunogenicity of both candidates was evaluated by delivery using the NP-1 formulation in an intramuscular injection and measurement of neutralizing antibodies (nAbs) by 80% plaque reduction neutralization test (PRNT80) against the US/MO/14-18947 isolate 14 days after the 10 micrograms (μg) prime and boost immunizations (FIG. 4D). Both candidates induced robust nAb titers with the IRES version trending better after the boost.

### Example 6: Construction of Replicon RNAs

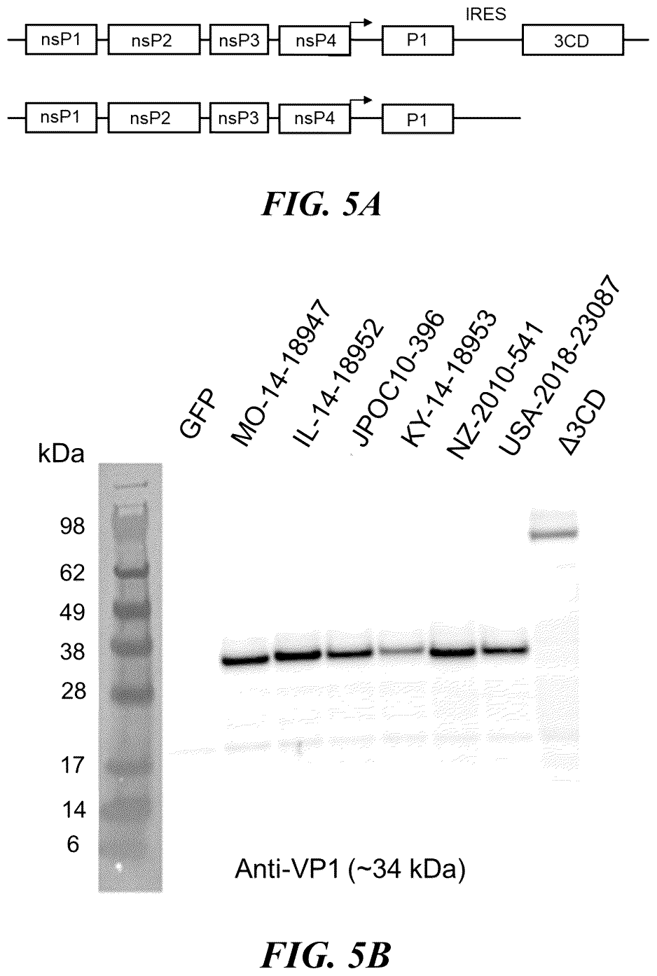

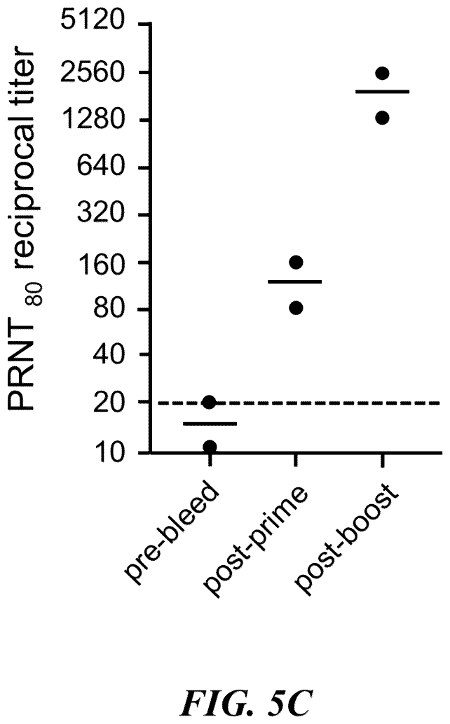

Using the IRES strategy from Example 5, six clade-specific versions, as well as a 3CD deletion mutant (FIG. 5A) were constructed and transfected in vitro. Correct processing of VP1 was confirmed using Western blot (FIG. 5B). Results show co-expression of 3CD is required for processing. Six repRNAs were combined as a mixture and alpacas were immunized with a 25 microgram (μg) dose using the NP-1 formulation. Neutralizing antibody (nAb) titers were measured 2 weeks after the prime and 2 weeks after the boost immunizations (FIG. 5C). Each animal responded to the prime immunization with PRNT₈₀ titers of 1:80 and 1:160 and, following the boost immunization, 1:1280 and 1:2560.

### Example 7: Selection of Candidate Therapeutic Antibodies

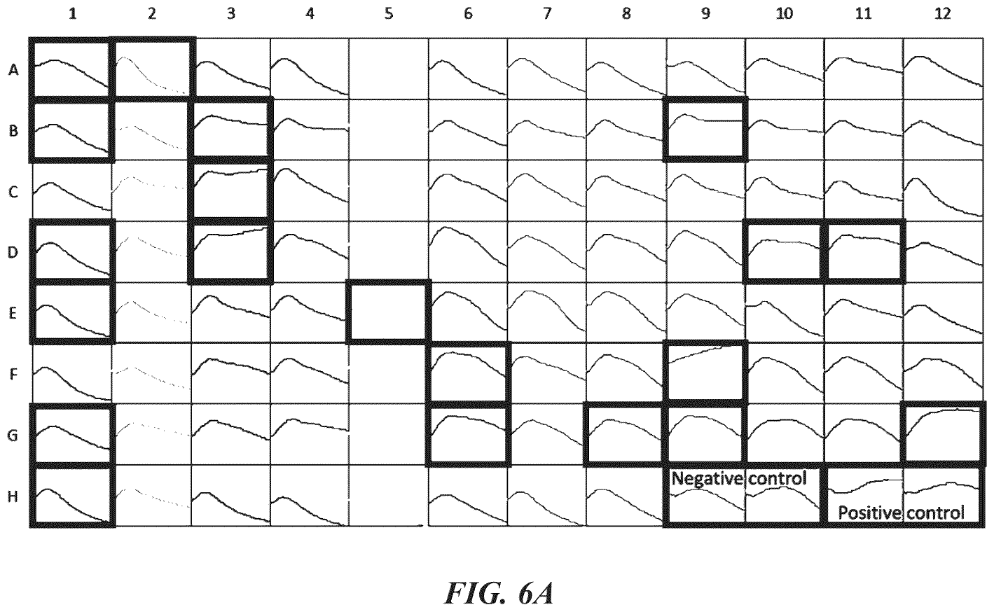

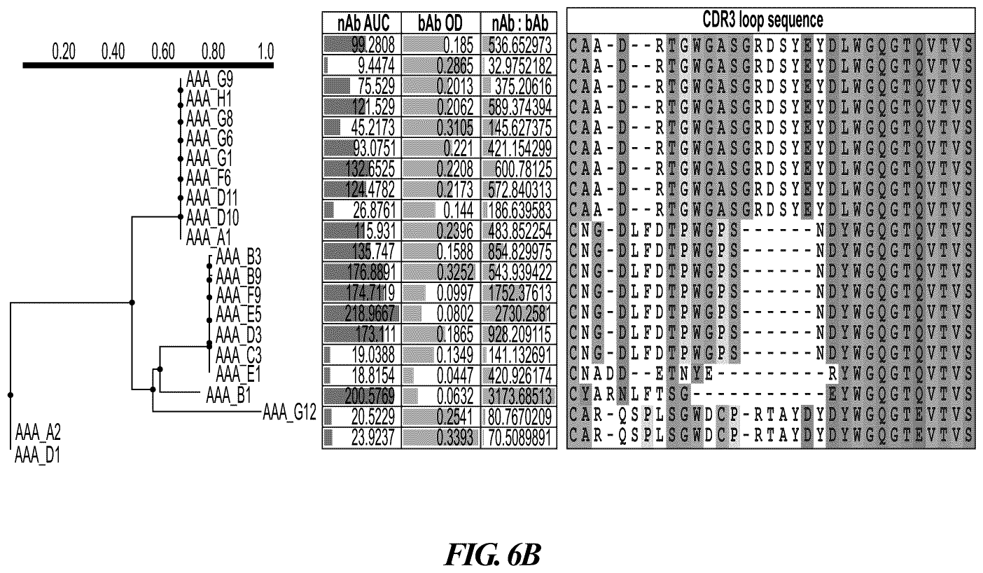

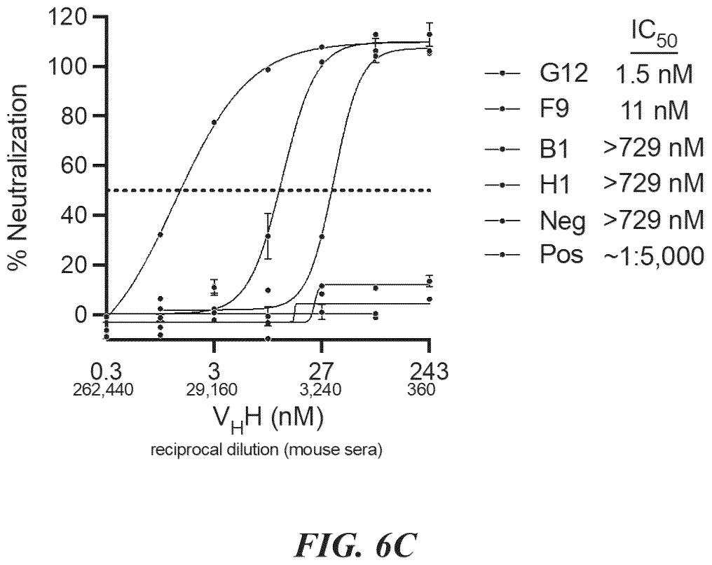

A high-throughput assay for rapid screening of neutralizing antibody activity was developed, comprising a cell impedance-based assay to monitor and quantify EV-D68-mediated cell morphology changes on monolayers of rhabdomyosarcoma (RD) cells over time in each well of a 96-well plate. This highly reproducible, virus-agnostic method was previously utilized for screening of human monoclonal antibodies against a variety of viral pathogens. The method was adapted for screening enriched phage libraries for EV-D68-neutralizing V_{H}Hs, we selected 92 colonies from an EV-D68-enriched phage library and induced expression of encoded V_{H}Hs along with 2 negative control colonies in a 96-well microplate. Supernatants were then clarified by centrifugation and this crude preparation of phage library-expressed V_{H}Hs was incubated with EV-D68 (US/MO-14-18947) and then overlaid, with the appropriate antibiotic, onto a monolayer of RD cells seeded on ePlates (Agilent) the night before along with 2 no-virus positive controls. These 96 total samples were then monitored on an xCELLigence™ real time cell analysis multiplate reader (Agilent, Santa Clara, CA), with cell impedance data collections every 15 minutes over a 3-day period (FIG. 6A). At least 13 potential candidates were identified. To validate the method, 20 different V_{H}Hs with a range of area-under-the-curve (AUC) values falling roughly into three groups were selected: low (purple outline), medium (orange outline), and high (red outline) neutralizing activity. EV-D68 binding activity was evaluated by enzyme linked immunosorbent assay (ELISA) so that neutralizing antibody (nAb)-to-binding antibody (bAb) ratios could be calculated to normalize for variability in soluble V_{H}H concentration present in crude bacterial supernatants. Additionally, the 20 selected V_{H}H colonies were Sanger sequenced to determine the phylogenetic relationships between V_{H}Hs with different nAb and bAb profiles (FIG. 6B). These data suggest that out of the 20 selected V_{H}Hs, 5 different CDR3 families were identified, only one of which did not demonstrate any binding or neutralizing activity (AAA_B1 (SEQ ID NO: 1)), with clear patterns in measured neutralizing and binding activities observed in the remaining 4 families. One V_{H}H from each of the top 3 neutralizing CDR3 families (AAA_H1 (SEQ ID NO: 2), AAA_F9 (SEQ ID NO: 3), AAA_G12 (SEQ ID NO: 4)) was selected, as well as the 1 V_{H}H from the CDR3 family with no binding or neutralizing activity (AAA_B1) and cloned into an expression vector for production and purification of recombinant V_{H}Hs for further characterization. Serial dilutions of each purified V_{H}H were incubated with 400,000 plaque forming units of EV-D68 and overlaid on RD cells seeded in ePlates and cell index measured over 36 hours as described above. Percent neutralization, as measured by the AUC of the normalized cell index over time, of each serial dilution was then plotted as a function of V_{H}H concentration so that 50% inhibitory concentrations could be determined (FIG. 6C).

### Example 8: Nanoparticle Delivery of DNA

The assay assessed delivery of various nanoparticles having DNA or RNA admixed therewith. Briefly, DNA encoding secreted embryonic alkaline phosphatase (SEAP) or replicon RNA encoding an RNA polymerase and SEAP were prepared and mixed with a nanoparticle of NP-1 or NP-3. Conditions are provided in Table 4. BALB/c female mice were injected intramuscularly (IM). Nucleic acid preparations for dilutions are provided in Table 5. Nanoparticle preparations are provided in Table 6. Nucleic acid-nanoparticle complexes were formed by adding 150 μl diluted NP-1 or NP-3 to 150 μl diluted DNA or RNA, then incubated for at least 30 minutes.

TABLE 4

| | | | | | | | Inj. | |
|---|---|---|---|---|---|---|---|---|
| | | For- | | RNA | DNA | | Vol- | |
| | | mula- | DNA/RNA- | dose | dose | | ume | |
| Group | N | tion | SEAP | [μg] | [ug] | N:P | [μl] | Route |
| 1 | 5 | Naked | DNA-SEAP | | 20 | n/a | 50 | IM |
| 2 | 5 | NP-1 | DNA-SEAP | | 10 | 15 | 50 | IM |
| 3 | 5 | NP-1 | DNA-SEAP | | 10 | 7.5 | | IM |
| 4 | 5 | NP-1 | DNA-SEAP | | 20 | 15 | | IM |
| 5 | 5 | NP-1 | DNA-SEAP | | 20 | 7.5 | | IM |
| 6 | 5 | NP-1 | RNA-SEAP | 1 | | 15 | 50 | IM |
| 7 | 5 | NP-3 | RNA-SEAP | 1 | | | 50 | IM |

TABLE 5

| | | | 40% | | | Concentrations | |
|---|---|---|---|---|---|---|---|
| | | DNA or | su- | | | measure prior | |
| | DNA- or | RNA | crose | water | Total | to complexing | |
| Group | RNA-SEAP | [μl] | [μl] | [μl] | [μl] | using NanoDrop | |
| 1 | DNA-SEAP | 24.0 | 75.0 | 51.0 | 150.0 | 725 | ug/ml |
| 2 | DNA-SEAP | 12.0 | 0.0 | 138.0 | 150.0 | 528 | ug/ml |
| 3 | DNA-SEAP | 12.0 | 0.0 | 138.0 | 150.0 | 528 | ug/ml |
| 4 | DNA-SEAP | 24.0 | 75.0 | 51.0 | 150.0 | 725 | ug/ml |
| 5 | DNA-SEAP | 24.0 | 75.0 | 51.0 | 150.0 | 725 | ug/ml |
| 6 | RNA-SEAP | 2.7 | 0.0 | 147.3 | 150.0 | 57 | ug/ml |
| 7 | RNA-SEAP | 2.7 | 0.0 | 147.3 | 150.0 | 57 | ug/ml |

TABLE 6

| | | | 40% | 100 mM | | |
|---|---|---|---|---|---|---|
| | | NP-1 | sucrose | citrate | Water | Total |
| Group | Formulation | [μl] | [μl] | [μl] | [μl] | [μl] |
| 1 | Naked | 0 | 0 | 15 | 135 | 150 |
| 2 | 100-015 | 72 | 90 | 18 | 0 | 180 |
| 3 | 100-015 | 36 | 90 | 18 | 36 | 180 |
| 4 | 100-015 | 144 | 0 | 18 | 18 | 180 |
| 5 | 100-015 | 72 | 0 | 18 | 90 | 180 |
| 6 | 100-015 | 7.2 | 90 | 18 | 64.8 | 180 |
| | | | | | | 180 |

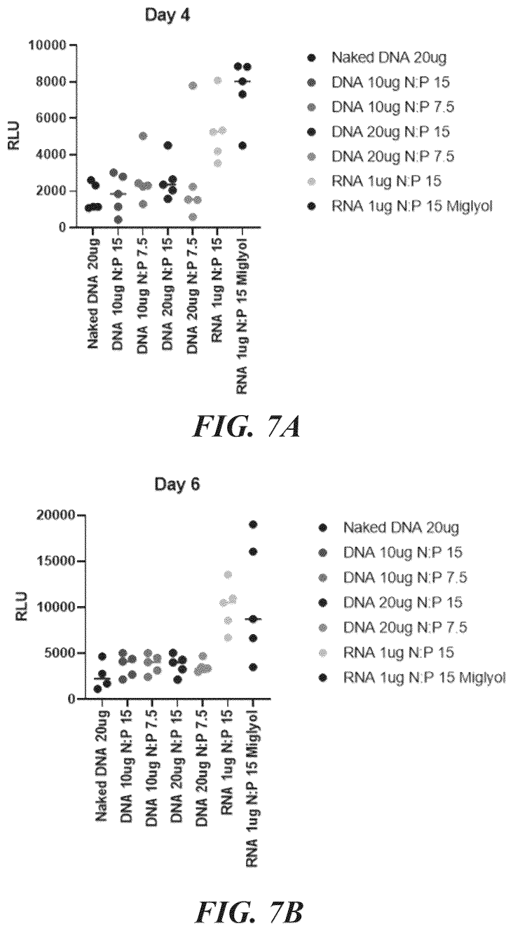

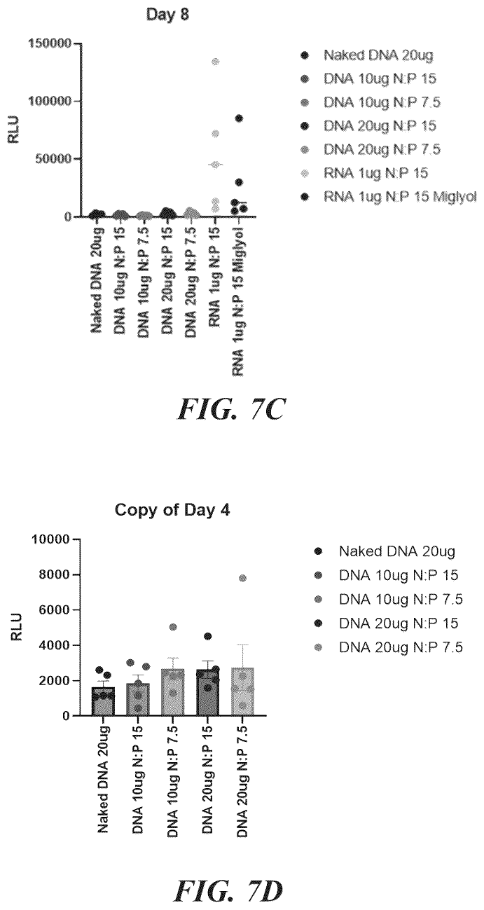

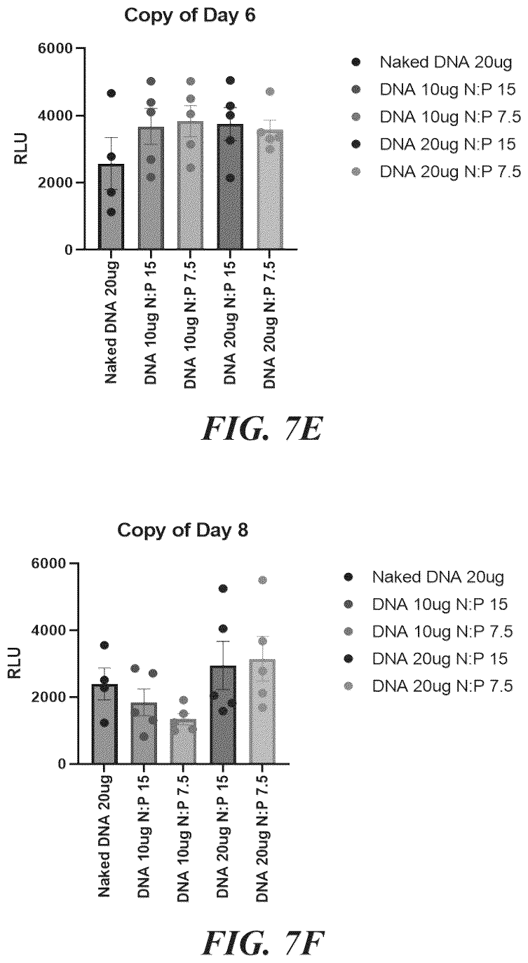

Mice were inoculated on day 0 according to the treatment groups. Blood was collected on days 4, 6 and 8, allowed to clot, and the serum was collected and stored at minus 80 degrees Celsius. Serum samples were thawed and SEAP detection was assessed. A chemiluminescent substrate of SEAP was provided, and activity was measured based on the light generated, and quantitated as Relative Luminescence Units (RLUs). Results are shown in FIGS. 7A-7F with a mean, n=5 per group. NP-1 and NP-3 formulations enhanced target protein production over delivery of DNA alone. Inclusion of Miglyol in NP-3 enhanced protein production of RNA over standard NP-1 having squalene.

### Example 9: Evaluation of Lyophilized Vaccines in Mice

The following was performed to assay activity of lyophilized NP-1 with replicon RNA encoded SARS-CoV-2 spike antigen sequence, physicochemical properties of reconstituted vaccines, potency, and immunogenicity. Briefly, materials in Table 7 were used.

TABLE 7
Materials.

| Name | Stock concentration | |
|---|---|---|
| NP-1 | 30 | mg/ml |
| | (measuring DOTAP conc.) | |
| NP-7 | 30 | mg/ml |
| | (measuring DOTAP conc.) | |
| repRNA-CoV2-spike | 1687 | μg/ml |
| (wild type) | | |
| VEE-S-v5 Delta | | |
| (“WT-S”) | | |
| repRNA-CoV2-spike | 783 | μg/ml |
| (delta) | | |
| VEE-nCOV19-S-Delta. | | |
| AY1-S2P-wtFur | | |
| (“Delta-S”) | | |
| Sucrose (EMD, | — | |
| Millipore) | | |
| Na-citrate (Teknova) | 1 | M |

Preparation of formulation complexes. Compositions of lipid nanoparticle/RNA complexes were prepared in this assay as shown below in Table 8. NP-1 or NP-7 and repRNAs were complexed at a N-to-P ratio of 15 and complexed to obtain a final repRNA concentration of 50 mg/ml or 100 mg/ml (“2×” material), and 10% or 20% w/v sucrose content, respectively. Complexed material with 10% sucrose (50 mg/ml repRNA) contained 5 mM sodium citrate while that with 20% sucrose (100 mg/ml repRNA) contained 10 mM citrate. Complexes were filled in 2 ml sterile, depyrogenated and baked vials. Complexes with 10% sucrose were filled at 0.7 ml per vial and 20% sucrose at 0.35 ml per vial. Vials were then either lyophilized and stored or stored as is in liquid form. Storage temperature was 25 degrees Celsius or 42 degrees Celsius for 1 week. Quantity of lyophilized and liquid vials per composition is summarized in Table 8.

TABLE 8
Formulations and Characteristics.

| | | DOTAP | RNA | Volume per | Lyo | Liquid |
|---|---|---|---|---|---|---|
| Description | N:P | [μg/ml] | [μg/ml] | vial [ml] | vials | vials |
| NP-1 + WT-S | 15 | 1500 | 50 | 0.7 | 8 | 6 |
| in 10% sucrose | | | | | | |
| NP-1 + Delta-S | 15 | 1500 | 50 | 0.7 | 2 | 0 |
| in 10% sucrose | | | | | | |
| NP-1 + WT-S | 15 | 3000 | 100 | 0.35 | 8 | 0 |
| in 20% sucrose | | | | | | |
| NP-1 + Delta-S | 15 | 3000 | 100 | 0.35 | 2 | 0 |
| in 20% sucrose | | | | | | |
| NP-7 + WT-S | 15 | 1500 | 50 | 0.7 | 8 | 6 |
| in 10% sucrose | | | | | | |

Lyophilization cycle. An SP VirTis Advantage Pro tray and batch lyophilizer with inert gas fill and stoppering capability was used. Summary of the lyophilization cycle is shown in Table 9 below. After end of cycle, vials were backfilled with nitrogen at 48 torr and stoppered, before equilibrating to room pressure.

TABLE 9
Conditions.

| Time | Temp | Pressure | |
|---|---|---|---|
| [hours] | [° C.] | [mT] | Notes |
| 0 | 5 | 760 | Shelf pre-cooled to 5 degrees C. |
| 0.5 | 5 | 760 | Freezing |
| 2 | −50 | 760 | |
| 2.5 | −50 | 50 | Evacuation |
| 3 | −30 | 50 | Primary drying |
| 20.5 | −30 | 50 | |
| 22.5 | 25 | 50 | Secondary drying |
| 24 | 25 | 50 | |

Condition groups. A summary of 14 groups analyzed in this assay is provided in Table 10 below. Groups 1 and 4, as indicated in the storage column, were prepared fresh to serve as positive controls for comparison with standard protocol for vaccine preparation.

TABLE 10
Conditions.

| | | | Sucrose | | Storage |
|---|---|---|---|---|---|
| Group | Formulation | RNA | [% w/v] | Form | [temp/time] |
| 1 | NP-1 | WT-S | 10 | Liquid | Fresh |
| 2 | NP-1 | WT-S | 10 | Liquid | 25° C./1 wk |
| 3 | NP-1 | WT-S | 10 | Liquid | 42° C./1 wk |
| 4 | NP-7 | WT-S | 10 | Liquid | Fresh |
| 5 | NP-7 | WT-S | 10 | Liquid | 25° C./1 wk |
| 6 | NP-7 | WT-S | 10 | Liquid | 42° C./1 wk |
| 7 | NP-1 | WT-S | 10 | Lyo | 25° C./1 wk |
| 8 | NP-1 | WT-S | 10 | Lyo | 42° C./1 wk |
| 9 | NP-1 | WT-S | 20 | Lyo | 25° C./1 wk |
| 10 | NP-1 | WT-S | 20 | Lyo | 42° C./1 wk |
| 11 | NP-7 | WT-S | 10 | Lyo | 25° C./1 wk |
| 12 | NP-7 | WT-S | 10 | Lyo | 42° C./1 wk |
| 13 | NP-1 | Delta-S | 10 | Lyo | 25° C./1 wk |
| 14 | NP-1 | Delta-S | 20 | Lyo | 25° C./1 wk |

Immunogenicity assay. Induction of andi-spike IgG responses were evaluated in 6 to 8 weeks old female C57BV/6 mice. A group size of 5 mice was used. The schedule is shown in Table 11.

TABLE 11
Immunogenicity Schedule.

| Date | Day | Procedure |
|---|---|---|
| Aug. 23, 2021 | −7 | Lyophilization |
| Sep. 1, 2021 | 0 | Immunization by IM route |
| Sep. 15, 2021 | 14 | Bleed |
| Sep. 29, 2021 | 28 | Bleed |
| Oct. 8, 2021 | 37 | Mice sacrificed |

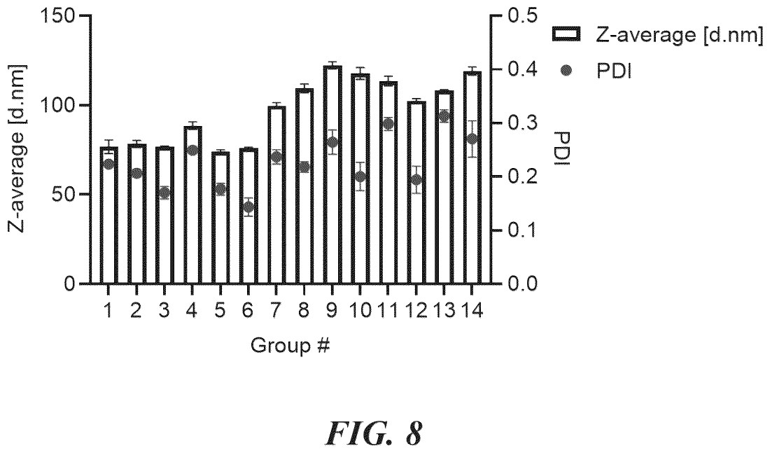

After 1 week of storage in 25 degrees Celsius or 42 degrees Celsius stability chamber, lyophilized nanoparticle/RNA complexes were reconstituted in 0.7 ml sterile milliQ water and gently swirled until no particles were visible to the naked eye. Particle size (z-average) and size distribution (PDI) of the complexes was measured and is summarized in FIG. 8, with group designations shown in Table 12. Particle size and PDI of freshly prepared NP-1/WT-S complex (group 1) was 76.8 nm and 0.223, respectively. After reconstitution, lyophilized samples (groups 7-14) grew by an average of 45% (+/−11%). Summary of % change in z-average relative to group 1 is included in Table 12.

TABLE 12
Percent % Change in Z-Average.

| Group # | 2 | 3 | 4 | 5 | 6 | 7 | 8 | 9 | 10 | 11 | 12 | 13 | 14 |
|---|---|---|---|---|---|---|---|---|---|---|---|---|---|
| % change | 2% | 0% | 15% | −4% | −1% | 30% | 42% | 59% | 53% | 48% | 33% | 41% | 55% |
| z-average | | | | | | | | | | | | | |
| vs. group 1 | | | | | | | | | | | | | |

Agarose gel electrophoresis of phenol-chloroform extracted repRNA. Liquid formulations of NP-1/repRNA and NP-7+repRNA in 10% sucrose or 20% sucrose, stored for 1 week at NP-1/repRNA and NP-7+repRNA, resulted in partial or full degradation of repRNA product, respectively. (Data not shown.) Lyophilization of NP-1/repRNA and NP-7+repRNA in 10% sucrose or 20% sucrose preserved repRNA integrity after 1 week storage at NP-1/repRNA and NP-7+repRNA. (Data not shown.)

Potency Assay. Lyophilized NP-1/WT-S in 10% sucrose stored for 1 week at 25 degrees Celsius produced a dose-dependent expression of spike protein in transfected BHK cells. The expression profile was similar to freshly complexed NP-1/WT-S. 1 week storage at 42 degrees Celsius of lyophilized NP-1/WT-S in 10% sucrose significantly reduced in vitro protein expression. Liquid NP-1/WT-S in 10% sucrose stored for 1 week at 25 degrees Celsius or 42 degrees Celsius did not produce spike protein in BHK cells. (Data not shown.)

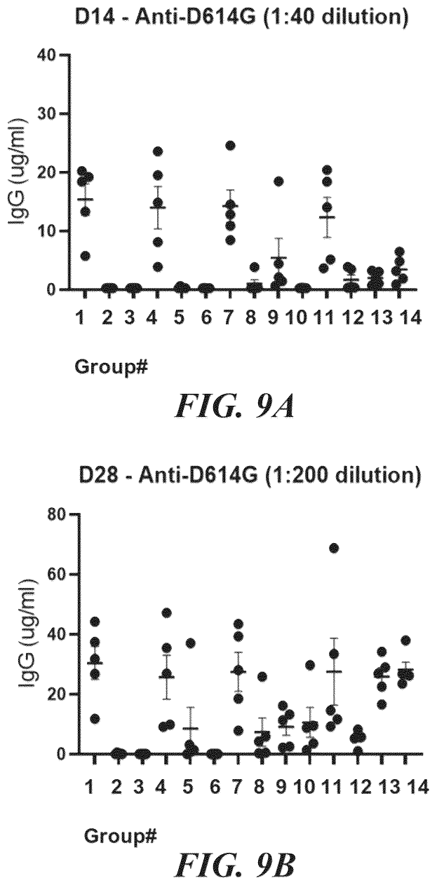

Anti-D614G spike IgG responses by ELISA. Serum anti-D614G spike IgG levels was assessed on days 14 and 28 post-prime shown below in FIGS. 9A and 9B, respectively. Mouse sera were assayed in an anti-D614G spike ELISA at 1:40 (day 14) or 1:200 (day 28) dilution. Serum IgG level in μg/ml was interpolated from a 4 parameter logistic (4PL) standard curve generated by a known concentration of mouse IgG standard.

Day 28 post-prime anti-D614G IgG response. After 1 week at 25 degrees C., liquid NP-1/WT-S in 10% sucrose resulted in a 3 statistically significant reduction in anti-spike IgG compared to the freshly prepared NP-1/WT-S positive control. There was no significant difference in mean IgG levels between freshly prepared NP-1/WT-S and lyophilized NP-1/WT-S in 10% sucrose stored for 1 week at 25 degrees C.

After 1 week at 42 degrees C., lyophilized NP-/WT-S in 10% sucrose induced 100% seroconversion but mean IgG level was significantly reduced compared to freshly prepared NP-1/WT-S. Summary mean+/−standard deviation IgG concentration data from day 28 post-immunization, including p-values determined by ordinary one-way ANOVA comparing against the freshly prepared NP-1/WT-S positive control, shown in Table 13. P<0.05 are considered statistically significant differences.

TABLE 13
Mean IgG at 1:200 Serum Dilution.

| | | | | | Storage | D 28 mean IgG | | P-value |
|---|---|---|---|---|---|---|---|---|
| | | | Sucrose | | temp. | at 1:200 serum | SD | vs. |
| Group | Formulation | RNA | [% w/v] | State | and time | dilution [μg/ml] | [μg/ml] | group 1 |
| 1 | NP-1 | WT-S | 10 | Liquid | Fresh | 30.38 | 12.29 | n/a |
| 2 | NP-1 | WT-S | 10 | Liquid | 25 C./1 wk | 0.17 | 0.23 | 0.0014 |
| 3 | NP-1 | WT-S | 10 | Liquid | 42 C./1 wk | 0.02 | 0.02 | 0.0013 |
| 4 | NP-7 | WT-S | 10 | Liquid | Fresh | 25.69 | 16.45 | 0.9990 |
| 5 | NP-7 | WT-S | 10 | Liquid | 25 C./1 wk | 8.47 | 15.99 | 0.0385 |
| 6 | NP-7 | WT-S | 10 | Liquid | 42 C./1 wk | 0.00 | 0.00 | 0.0013 |
| 7 | NP-1 | WT-S | 10 | Lyo | 25 C./1 wk | 27.44 | 14.68 | 0.9994 |
| 8 | NP-1 | WT-S | 10 | Lyo | 42 C./1 wk | 7.34 | 10.63 | 0.0257 |
| 9 | NP-1 | WT-S | 20 | Lyo | 25 C./1 wk | 9.07 | 6.41 | 0.0474 |
| 10 | NP-1 | WT-S | 20 | Lyo | 42 C./1 wk | 10.56 | 11.25 | 0.0777 |
| 11 | NP-7 | WT-S | 10 | Lyo | 25 C./1 wk | 27.53 | 24.96 | 0.9994 |
| 12 | NP-7 | WT-S | 10 | Lyo | 42 C./1 wk | 5.33 | 2.68 | 0.0121 |
| 13 | NP-1 | Delta-S | 10 | Lyo | 25 C./1 wk | 25.83 | 6.66 | 0.9990 |
| 14 | NP-1 | Delta-S | 20 | Lyo | 25 C./1 wk | 28.25 | 5.60 | 0.9996 |

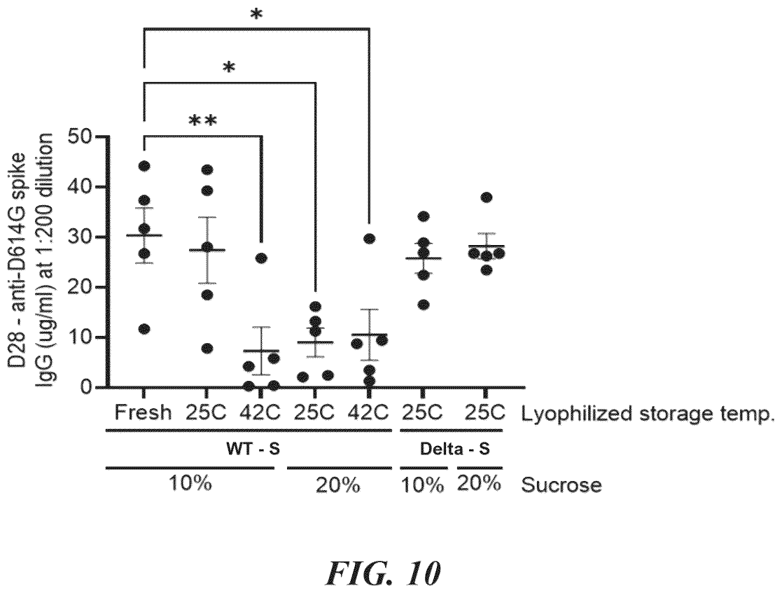

Comparison of fresh versus lyophilized formulations. Day 28 post-prime anti-D614G spike IgG concentration in serum is shown in FIG. 10. Statistical differences between mean IgG values were determined by ordinary one-way ANOVA with Dunnett's multiple comparisons test. All groups compared to freshly prepared NP-1/RNA in 10% sucrose. No significant difference was shown between freshly prepared NP-1/RNA and lyophilized NP-1/RNA in 10% sucrose stored for 7 days at 25 degrees Celsius. At 42 degrees C., lyophilized NP-1/RNA in 10% or 20% sucrose induced significantly lower anti-spike IgG compared to freshly prepared NP-1/RNA. Lyophilized NP-1/RNA in 20% sucrose, and stored at 25 degrees Celsius or 42 degrees C., induced significantly lower IgG than freshly prepared NP-1/WT-S. Lyophilized NP-1/Delta-S in 10% or 20% sucrose, and stored at 25 degrees C., induced similar mean IgG (statistically not significant) than freshly prepared NP-1/WT-S.

### Example 10: Fusion with Fc Domain of Human IgG1 Enhances Breadth of Neutralization

Recombinant V_{H}H G12, which was identified in the phage-display library after panning against clade B1 enterovirus D68 (EV-D68). The construct was expressed in and purified from E. coli and then assayed for neutralizing activity against all 6 genotypes of EV-68 by real time cell analysis (RTCA) assay.

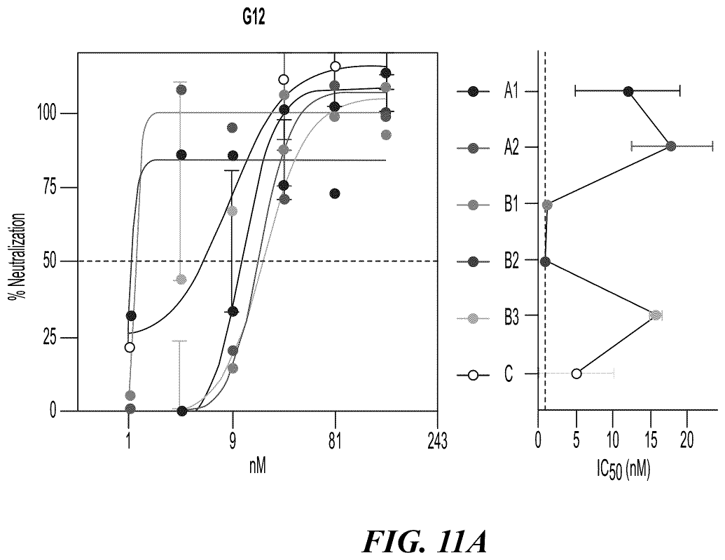

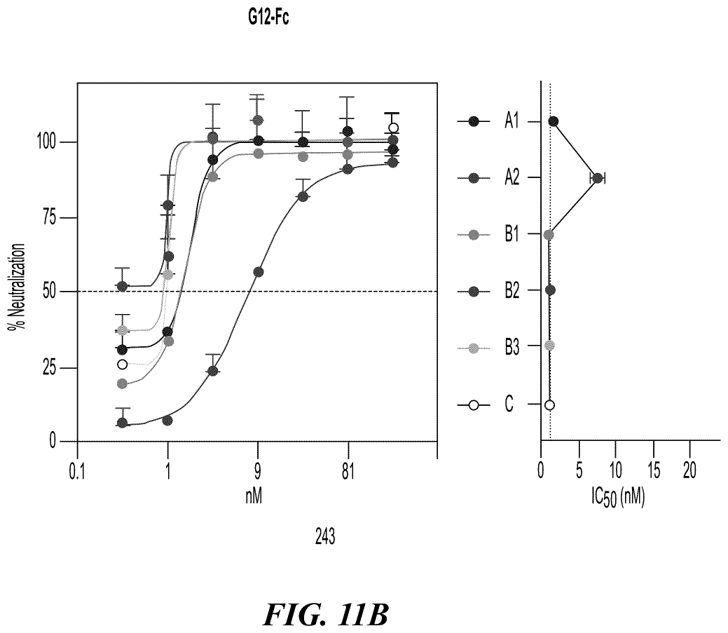

While 50% inhibitory concentrations (IC₅₀) where low (˜1 nM) for homologous virus (clade B1) and the closely related clade B2 virus, significant loss of neutralizing activity was observed against clades A1, A2, B3, and C, with 5-20-fold reductions in potency (FIG. 11A). V_{H}H G12 was then fused with the Fc domain of human IgG1 (SEQ ID NO: 12). SEQ ID NO: 12 was then expressed in and purified from E. coli, and assayed for neutralizing activity against all 6 genotypes of EV-68 by real time cell analysis (RTCA) assay (FIG. 11B). In contrast to the G12 monomer, the G12-Fc dimer exhibited enhanced breadth of neutralization with an IC₅₀ between 0.8 and 1.4 nM against clades A1, B1, B2, B3, and C and approximately 7 nM against clade A2. Therefore, G12-Fc had approximately a 2-fold increase in potency relative to the G12 monomer measured against A2 virus.

### Example 11: Additional EV-D68 repRNA Constructs

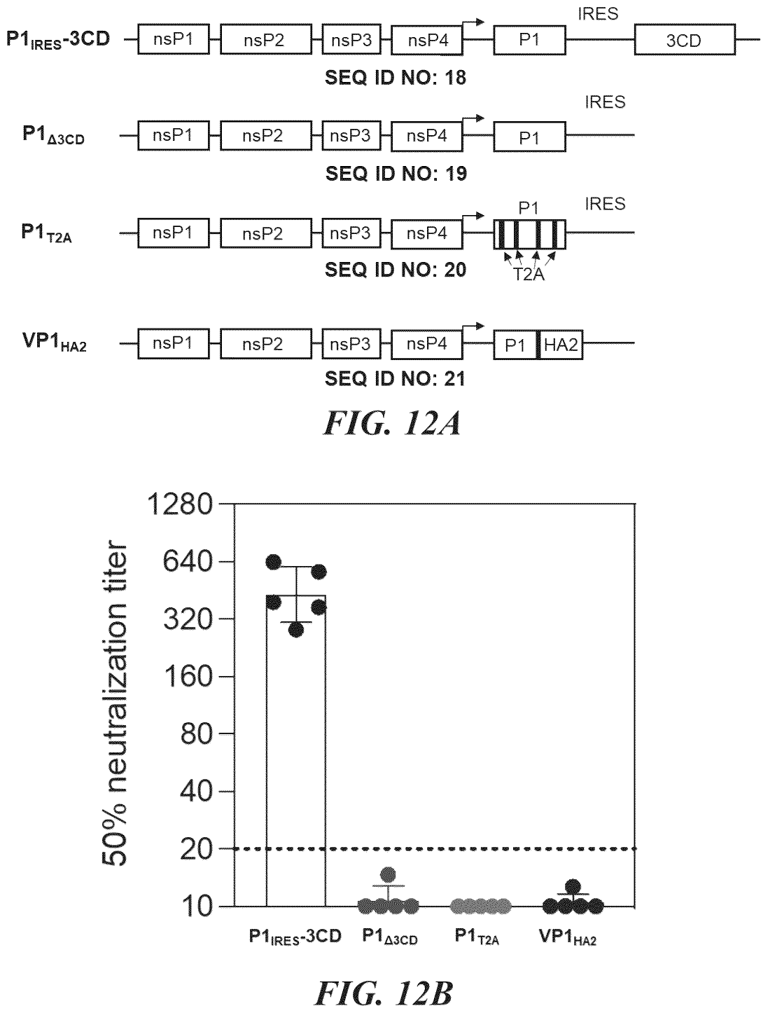

Four RNA constructs were generated as provided in Table 14 below and shown in FIG. 12A.

TABLE 14
EV-D68 repRNA Constructs.

| Construct | SEQ ID NO: | Elements |
|---|---|---|
| P1_{IRES}-3CD | 18 | Full-length polyprotein |
| P1_{Δ3CD} | 19 | 3CD deleted |
| P1_{T2A} | 20 | T2A-separated capsid subunits |
| VP1_{HA2} | 21 | VP1 fused to influenza HA (domain 2) |

VP1 HA2 was generated with hemagglutinin A from influenza, an enveloped virus, to secrete VP1 proteins. The constructs were each expressed in and purified from E. coli and then assayed for neutralizing activity against EV-68 by real time cell analysis (RTCA) assay.

Neutralization titers showed that constructs that did not have a 3CD protease did not neutralize EV-D68. The full-length polyprotein with 3CD protease was necessary for inducing neutralizing antibodies to EV-D68 (FIG. 12B).

### Example 12: Non-Human Primates Vaccinated with EV-D68 B1 Vaccine Neutralize B1 Virus

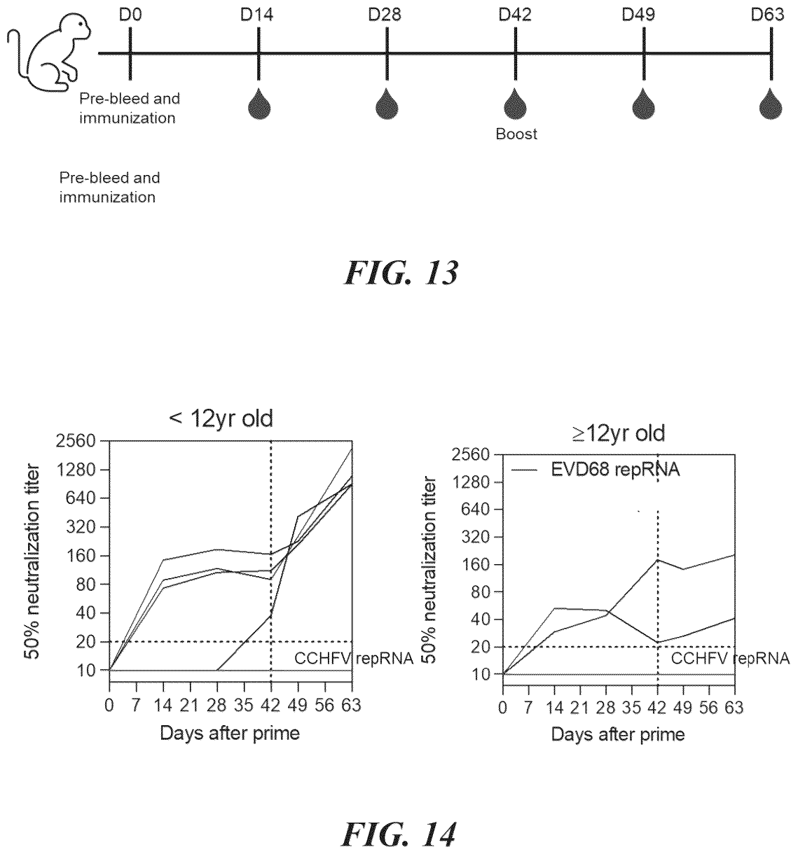

Six non-human primates were immunized with EV-D68 B1 repRNA vaccine (SEQ ID NO: 18) and blood was drawn according to the schedule in FIG. 13. Controls were immunized with Crimean-Congo Hemorrhagic Fever Virus (CCHFV) repRNA vaccine. Sera from immunized animals was obtained for neutralization assays. Neutralization was measured by xCELLigence™ as described in Example 7.

Non-human primates vaccinated with EV-D68 Bi repRNA vaccine produced 50% neutralization titers within 7 days after vaccination and neutralized the B1 enterovirus (FIG. 14).

SEQUENCES

SEQ ID NOS: 1-4: See Table 1-Anti-EV-D68 (VHH) Amino Acid Sequences
SEQ ID NOS: 5-7: See Table 2-EV-D68 Binding CDR3 Loop Sequences
SEQ ID NO: 8: VEEV RNA Sequence
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacquaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgccaag
SEQ ID NO: 9: VEEV RNA polymerase Amino Acid Sequence (NCBI Accession: AXP98866.1)
RELPVLDSAAFNVECEKKYACNNEYWETFKENPIRLTEENVVNYITKLKGP
SEQ ID NO: 10: VEEV RNA polymerase Amino Acid Sequence (NCBI Accession: AXP98867.1)
TQMRELPVLDSAAFNVECFKKYACNNEYWETFKENPIRLTE
SEQ ID NO: 11: Polyprotein Amino Acid Sequence [Venezuelan equine encephalitis virus]
(GenBank: ALE15116.1)
MKAITARRILQGLGHYLKAEGKVECYRTLHPVPLYSSSVNRAFSSPKVAVEACNAMLKENFPTVASYCII
PEYDAYLDMIDGASCCLDTASFCPAKLRSFPKKHSYLEPTIRSAVPSAIQNTLQNVLAAATKRNCNVTQM
RELPVLDSAAFNVECFKKYACNNEYWKTFKENPIRLTEENVINYITKLKGPKAAALYAKTHNLNMLQDIP
MDRFVMDLKRDVKVTPGTKHTEERPKVQVIQAADPLATAYLCGIHRELVRRLNAVLLPNIHTLEDMSAED
FDAIIAEHFQPGDCVLETDIASFDKSEDDAMALTAMMILEDLGVDAELLTLIEAAFGEISSIHLPTKTKF
KFGAMMKSGMFLTLFVNTVINIVIASRVLRERLTGSPCAAFIGDDNIVKGVKSDKLMADRCATWLNMEVK
IIDAVVGEKAPYFCGGFILCDSVTGTACRVADPLKRLFKLGKPLAADDEHDDDRRRALHEESTRWNRVGI
LPELCKAVESRYETVGTSVIVMAMATLASSVKSFSYLRGAPITLYG
SEQ ID NOS: 12 and 13 have been formatted as follows:
VEE sequence: lowercase
Gene of interest start codon: Italicized CAPS
Signal peptide: Bold Italicized CAPS
V_{H}H sequence: Non-underlined CAPS
Fc: Bolded CAPS
SEQ ID NO: 12: Replicon RNA Sequence Encoding 717-VEE-EV-D68-V_{H}H-F9-FC
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacquaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgccgccaccAU
GGAGUUCGGUCUUAGCUGGGUGUUUCUUGUCGCCCUGUUCAGAGGGGUACAAUGCGGCCCGGGAGCGGCCGCUCAGUU
GCAGCUGGUGGAGUCAGGUGGAGGCUUGGUGCAGCCUGGGGGGUCUCUGAGACUCUCCUGUGCAGCCUCUGGCCGCGU
CAUCGGAAUCAAUGCCAUGGGCUGGUACCGCCAGGCUCCAGGGAAGCAGCGCGAGUUGGUCGCACGAGUUACUCAAGC
UGGUAACAUCAACUAUGCAGACUCCGUGAAGGACCGAUUCACCAUCUCCAGAGACAAGGCCGAGAACGCGGUGUAUCU
ACAAAUGAACAGCCUCAAACCUGAGGACACGGCCGUCUACUACUGUAAUGGAGAUCUUUUCGAUACGCCUUGGGGUCC
AUCAAAUGACUACUGGGGCCAGGGGACCCAAGUCACCGUCUCCUCAGACAAAACUCACACAUGCCCACCGUGCCCAGC
ACCUGAACUCCUGGGGGGACCGUCAGUCUUCCUCUUCCCCCCAAAACCCAAGGACACCCUCAUGAUCUCCCGGACCCC
UGAGGUCACAUGCGUGGUGGUGGACGUGAGCCACGAAGACCCUGAGGUCAAGUUCAACUGGUACGUGGACGGCGUGGA
GGUGCAUAAUGCCAAGACAAAGCCGCGGGAGGAGCAGUACAACAGCACGUACCGUGUGGUCAGCGUCCUCACCGUCCU
GCACCAGGACUGGCUGAAUGGCAAGGAGUACAAGUGCAAGGUCUCCAACAAAGCCCUCCCAGCCCCCAUCGAGAAAAC
CAUCUCCAAAGCCAAAGGGCAGCCCCGAGAACCACAGGUGUACACCCUGCCCCCAUCCCGGGAGGAGAUGACCAAGAA
CCAGGUCAGCCUGACCUGCCUGGUCAAAGGCUUCUAUCCCAGCGACAUCGCCGUGGAGUGGGAGAGCAAUGGGCAGCC
GGAGAACAACUACAAGACCACGCCUCCCGUGCUGGACUCCGACGGCUCCUUCUUCCUCUACAGCAAGCUCACCGUGGA
CAAGAGCAGGUGGCAGCAGGGGAACGUCUUCUCAUGCUCCGUGAUGCAUGAGGCUCUGCACAACCACUACACGCAGAA
GAGCCUCUCCCUGUCUCCGGGUAAAugauaaccgcggugucaaaaaccgcguggacgugguuaacaucccugcuggga
ggaucagccguaauuauuauaauuggcuuggugcuggcuacuauuguggccauguacgugcugaccaaccagaaacau
aauugaauacagcagcaauuggcaagcugcuuacauagaacucgcggcgauuggcaugccgccuuaaaauuuuuauuu
uauuuuuucuuuucuuuuccgaaucggauuuuguuuuuaauauuucaaaaaaaaaaaaaaaaaaaaaaaaaaaaaaaa
aaaaaaaaaaaaa
SEQ ID NO: 13-Replicon RNA sequence 718-VEE-EV-D68-VHH-G12-FC
auaggcggcgcaugagagaagcccagaccaauuaccuacccaaaauggagaaaguucacguugacaucgaggaagaca
gcccauuccucagagcuuugcagcggagcuucccgcaguuugagguagaagccaagcaggucacugauaaugaccaug
cuaaugccagagcguuuucgcaucuggcuucaaaacugaucgaaacggagguggacccauccgacacgauccuugaca
uuggaagugcgcccgcccgcagaauguauucuaagcacaaguaucauuguaucuguccgaugagaugugcggaagauc
cggacagauuguauaaguaugcaacuaagcugaagaaaaacuguaaggaaauaacugauaaggaauuggacaagaaaa
ugaaggagcuggccgccgucaugagcgacccugaccuggaaacugagacuaugugccuccacgacgacgagucguguc
gcuacgaagggcaagucgcuguuuaccaggauguauacgcgguugacggaccgacaagucucuaucaccaagccaaua
agggaguuagagucgccuacuggauaggcuuugacaccaccccuuuuauguuuaagaacuuggcuggagcauauccau
cauacucuaccaacugggccgacgaaaccguguuaacggcucguaacauaggccuaugcagcucugacguuauggagc
ggucacguagagggauguccauucuuagaaagaaguauuugaaaccauccaacaauguucuauucucuguuggcucga
ccaucuaccacgagaagagggacuuacugaggagcuggcaccugccgucuguauuucacuuacguggcaagcaaaauu
acacaugucggugugagacuauaguuaguugcgacggguacgucguuaaaagaauagcuaucaguccaggccuguaug
ggaagccuucaggcuaugcugcuacgaugcaccgcgagggauucuugugcugcaaagugacagacacauugaacgggg
agagggucucuuuucccgugugcacquaugugccagcuacauugugugaccaaaugacuggcauacuggcaacagaug
ucagugcggacgacgcgcaaaaacugcugguugggcucaaccagcguauagucgucaacggucgcacccagagaaaca
ccaauaccaugaaaaauuaccuuuugcccguaguggcccaggcauuugcuaggugggcaaaggaauauaaggaagauc
aagaagaugaaaggccacuaggacuacgagauagacaguuagucaugggguguuguugggcuuuuagaaggcacaaga
uaacaucuauuuauaagcgcccggauacccaaaccaucaucaaagugaacagcgauuuccacucauucgugcugccca
ggauaggcaguaacacauuggagaucgggcugagaacaagaaucaggaaaauguuagaggagcacaaggagccgucac
cucucauuaccgccgaggacguacaagaagcuaagugcgcagccgaugaggcuaaggaggugcgugaagccgaggagu
ugcgcgcagcucuaccaccuuuggcagcugauguugaggagcccacucuggaggcagacgucgacuugauguuacaag
aggcuggggccggcucaguggagacaccucguggcuugauaaagguuaccagcuacgauggcgaggacaagaucggcu
cuuacgcugugcuuucuccgcaggcuguacucaagagugaaaaauuaucuugcauccacccucucgcugaacaaguca
uagugauaacacacucuggccgaaaagggcguuaugccguggaaccauaccaugguaaaguaguggugccagagggac
augcaauacccguccaggacuuucaagcucugagugaaagugccaccauuguguacaacgaacgugaguucguaaaca
gguaccugcaccauauugccacacauggaggagcgcugaacacugaugaagaauauuacaaaacugucaagcccagcg
agcacgacggcgaauaccuguacgacaucgacaggaaacagugcgucaagaaagaacuagucacugggcuagggcuca
caggcgagcugguggauccucccuuccaugaauucgccuacgagagucugagaacacgaccagccgcuccuuaccaag
uaccaaccauagggguguauggcgugccaggaucaggcaagucuggcaucauuaaaagcgcagucaccaaaaaagauc
uaguggugagcgccaagaaagaaaacugugcagaaauuauaagggacgucaagaaaaugaaagggcuggacgucaaug
ccagaacuguggacucagugcucuugaauggaugcaaacaccccguagagacccuguauauugacgaagcuuuugcuu
gucaugcagguacucucagagcgcucauagccauuauaagaccuaaaaaggcagugcucugcggggaucccaaacagu
gcgguuuuuuuaacaugaugugccugaaagugcauuuuaaccacgagauuugcacacaagucuuccacaaaagcaucu
cucgccguugcacuaaaucugugacuucggucgucucaaccuuguuuuacgacaaaaaaaugagaacgacgaauccga
aagagacuaagauugugauugacacuaccggcaguaccaaaccuaagcaggacgaucucauucucacuuguuucagag
ggugggugaagcaguugcaaauagauuacaaaggcaacgaaauaaugacggcagcugccucucaagggcugacccgua
aagguguguaugccguucgguacaaggugaaugaaaauccucuguacgcacccaccucagaacaugugaacguccuac
ugacccgcacggaggaccgcaucguguggaaaacacuagccggcgacccauggauaaaaacacugacugccaaguacc
cugggaauuucacugccacgauagaggaguggcaagcagagcaugaugccaucaugaggcacaucuuggagagaccgg
acccuaccgacgucuuccagaauaaggcaaacguguguugggccaaggcuuuagugccggugcugaagaccgcuggca
uagacaugaccacugaacaauggaacacuguggauuauuuugaaacggacaaagcucacucagcagagauaguauuga
accaacuaugcgugagguucuuuggacucgaucuggacuccggucuauuuucugcacccacuguuccguuauccauua
ggaauaaucacugggauaacuccccgucgccuaacauguacgggcugaauaaagaagugguccgucagcucucucgca
gguacccacaacugccucgggcaguugccacuggaagagucuaugacaugaacacugguacacugcgcaauuaugauc
cgcgcauaaaccuaguaccuguaaacagaagacugccucaugcuuuaguccuccaccauaaugaacacccacagagug
acuuuucuucauucgucagcaaauugaagggcagaacuguccugguggucggggaaaaguuguccgucccaggcaaaa
ugguugacugguugucagaccggccugaggcuaccuucagagcucggcuggauuuaggcaucccaggugaugugccca
aauaugacauaauauuuguuaaugugaggaccccauauaaauaccaucacuaucagcagugugaagaccaugccauua
agcuuagcauguugaccaagaaagcuugucugcaucugaaucccggcggaaccugugucagcauagguuaugguuacg
cugacagggccagcgaaagcaucauuggugcuauagcgcggcaguucaaguuuucccggguaugcaaaccgaaauccu
cacuugaagagacggaaguucuguuuguauucauuggguacgaucgcaaggcccguacgcacaauccuuacaagcuuu
caucaaccuugaccaacauuuauacagguuccagacuccacgaagccggaugugcacccucauaucauguggugcgag
gggauauugccacggccaccgaaggagugauuauaaaugcugcuaacagcaaaggacaaccuggcggaggggugugcg
gagcgcuguauaagaaauucccggaaagcuucgauuuacagccgaucgaaguaggaaaagcgcgacuggucaaaggug
cagcuaaacauaucauucaugccguaggaccaaacuucaacaaaguuucggagguugaaggugacaaacaguuggcag
aggcuuaugaguccaucgcuaagauugucaacgauaacaauuacaagucaguagcgauuccacuguuguccaccggca
ucuuuuccgggaacaaagaucgacuaacccaaucauugaaccauuugcugacagcuuuagacaccacugaugcagaug
uagccauauacugcagggacaagaaaugggaaaugacucucaaggaagcaguggcuaggagagaagcaguggaggaga
uaugcauauccgacgacucuucagugacagaaccugaugcagagcuggugagggugcauccgaagaguucuuuggcug
gaaggaagggcuacagcacaagcgauggcaaaacuuucucauauuuggaagggaccaaguuucaccaggcggccaagg
auauagcagaaauuaaugccauguggcccguugcaacggaggccaaugagcagguaugcauguauauccucggagaaa
gcaugagcaguauuaggucgaaaugccccgucgaagagucggaagccuccacaccaccuagcacgcugccuugcuugu
gcauccaugccaugacuccagaaagaguacagcgccuaaaagccucacguccagaacaaauuacugugugcucauccu
uuccauugccgaaguauagaaucacuggugugcagaagauccaaugcucccagccuauauuguucucaccgaaagugc
cugcguauauucauccaaggaaguaucucguggaaacaccaccgguagacgagacuccggagccaucggcagagaacc
aauccacagaggggacaccugaacaaccaccacuuauaaccgaggaugagaccaggacuagaacgccugagccgauca
ucaucgaagaggaagaagaggauagcauaaguuugcugucagauggcccgacccaccaggugcugcaagucgaggcag
acauucacgggccgcccucuguaucuagcucauccugguccauuccucaugcauccgacuuugauguggacaguuuau
ccauacuugacacccuggagggagcuagcgugaccagcggggcaacgucagccgagacuaacucuuacuucgcaaaga
guauggaguuucuggcgcgaccggugccugcgccucgaacaguauucaggaacccuccacaucccgcuccgcgcacaa
gaacaccgucacuugcacccagcagggccugcucgagaaccagccuaguuuccaccccgccaggcgugaauaggguga
ucacuagagaggagcucgaggcgcuuaccccgucacgcacuccuagcaggucggucucgagaaccagccuggucucca
acccgccaggcguaaauagggugauuacaagagaggaguuugaggcguucguagcacaacaacaaugacgguuugaug
cgggugcauacaucuuuuccuccgacaccggucaagggcauuuacaacaaaaaucaguaaggcaaacggugcuauccg
aagugguguuggagaggaccgaauuggagauuucguaugccccgcgccucgaccaagaaaaagaagaauuacuacgca
agaaauuacaguuaaaucccacaccugcuaacagaagcagauaccaguccaggaagguggagaacaugaaagccauaa
cagcuagacguauucugcaaggccuagggcauuauuugaaggcagaaggaaaaguggagugcuaccgaacccugcauc
cuguuccuuuguauucaucuagugugaaccgugccuuuucaagccccaaggucgcaguggaagccuguaacgccaugu
ugaaagagaacuuuccgacuguggcuucuuacuguauuauuccagaguacgaugccuauuuggacaugguugacggag
cuucaugcugcuuagacacugccaguuuuugcccugcaaagcugcgcagcuuuccaaagaaacacuccuauuuggaac
ccacaauacgaucggcagugccuucagcgauccagaacacgcuccagaacguccuggcagcugccacaaaaagaaauu
gcaaugucacgcaaaugagagaauugcccguauuggauucggcggccuuuaauguggaaugcuucaagaaauaugcgu
guaauaaugaauauugggaaacguuuaaagaaaaccccaucaggcuuacugaagaaaacgugguaaauuacauuacca
aauuaaaaggaccaaaagcugcugcucuuuuugcgaagacacauaauuugaauauguugcaggacauaccaauggaca
gguuuguaauggacuuaaagagagacgugaaagugacuccaggaacaaaacauacugaagaacggcccaagguacagg
ugauccaggcugccgauccgcuagcaacagcguaucugugcggaauccaccgagagcugguuaggagauuaaaugcgg
uccugcuuccgaacauucauacacuguuugauaugucggcugaagacuuugacgcuauuauagccgagcacuuccagc
cuggggauuguguucuggaaacugacaucgcgucguuugauaaaagugaggacgacgccauggcucugaccgcguuaa
ugauucuggaagacuuagguguggacgcagagcuguugacgcugauugaggcggcuuucggcgaaauuucaucaauac
auuugcccacuaaaacuaaauuuaaauucggagccaugaugaaaucuggaauguuccucacacuguuugugaacacag
ucauuaacauuguaaucgcaagcagaguguugagagaacggcuaaccggaucaccaugugcagcauucauuggagaug
acaauaucgugaaaggagucaaaucggacaaauuaauggcagacaggugcgccaccugguugaauauggaagucaaga
uuauagaugcuguggugggcgagaaagcgccuuauuucuguggaggguuuauuuugugugacuccgugaccggcacag
cgugccguguggcagacccccuaaaaaggcuguuuaagcuuggcaaaccucuggcagcagacgaugaacaugaugaug
acaggagaagggcauugcaugaagagucaacacgcuggaaccgaguggguauucuuucagagcugugcaaggcaguag
aaucaagguaugaaaccguaggaacuuccaucauaguuauggccaugacuacucuagcuagcaguguuaaaucauuca
gcuaccugagaggggccccuauaacucucuacggcuaaccugaauggacuacgacauagucuaguccgccgccaccAU
GGAGUUCGGUCUUAGCUGGGUGUUUCUUGUCGCCCUGUUCAGAGGGGUACAAUGCGGCCCGGGAGCGGCCGCUCAGGU
GCAGCUGGUGGAGUCGGGGGGAGGUUUGGUGCAGCCUGGAGGGUCUCUGAGACUCUCCUGUUUAGCCUCUGGAAUCAC
CUUCACUGUCUAUCGCAUGGCCUGGUACCGUCAGGCUCCGGGGAGGCAGCGCGACUUGGUCGCAGAAGUAGCUCCUGG
UGGUGGAACGGUGGCUGCAAACUCCGUGAAGGGCCGAUUCACCAUCUCCAGAGACAGCGCCAAGAACACGGUGGAUCU
GCAAAUGAACGACCUGAAACCUGACGAUACGGCCGUCUAUUAUUGUUAUGCACGUAAUCUUUUCACGUCGGGGGAGUA
UUGGGGCCAGGGGACCCAGGUCACCGUCUCCUCAGACAAAACUCACACAUGCCCACCGUGCCCAGCACCUGAACUCCU
GGGGGGACCGUCAGUCUUCCUCUUCCCCCCAAAACCCAAGGACACCCUCAUGAUCUCCCGGACCCCUGAGGUCACAUG
CGUGGUGGUGGACGUGAGCCACGAAGACCCUGAGGUCAAGUUCAACUGGUACGUGGACGGCGUGGAGGUGCAUAAUGC
CAAGACAAAGCCGCGGGAGGAGCAGUACAACAGCACGUACCGUGUGGUCAGCGUCCUCACCGUCCUGCACCAGGACUG
GCUGAAUGGCAAGGAGUACAAGUGCAAGGUCUCCAACAAAGCCCUCCCAGCCCCCAUCGAGAAAACCAUCUCCAAAGC
CAAAGGGCAGCCCCGAGAACCACAGGUGUACACCCUGCCCCCAUCCCGGGAGGAGAUGACCAAGAACCAGGUCAGCCU
GACCUGCCUGGUCAAAGGCUUCUAUCCCAGCGACAUCGCCGUGGAGUGGGAGAGCAAUGGGCAGCCGGAGAACAACUA
CAAGACCACGCCUCCCGUGCUGGACUCCGACGGCUCCUUCUUCCUCUACAGCAAGCUCACCGUGGACAAGAGCAGGUG
GCAGCAGGGGAACGUCUUCUCAUGCUCCGUGAUGCAUGAGGCUCUGCACAACCACUACACGCAGAAGAGCCUCUCCCU
GUCUCCGGGUAAAugauaaccgcggugucaaaaaccgcguggacgugguuaacaucccugcugggaggaucagccgua
auuauuauaauuggcuuggugcuggcuacuauuguggccauguacgugcugaccaaccagaaacauaauugaauacag
cagcaauuggcaagcugcuuacauagaacucgcggcgauuggcaugccgccuuaaaauuuuuauuuuauuuuuucuuu
ucuuuuccgaaucggauuuuguuuuuaauauuucaaaaaaaaaaaaaaaaaaaaaaaaaaaaaaaaaaaaaaaaaaaa
a
SEQ ID NO: 14: RNA encoding V_{H}H- 717
GGCCCGGGAGCGGCCGCUCAGUUGCAGCUGGUGGAGUCAGGUGGAGGCUUGGUGCAGCCUGGGGGGUCUCUGAGACUC
UCCUGUGCAGCCUCUGGCCGCGUCAUCGGAAUCAAUGCCAUGGGCUGGUACCGCCAGGCUCCAGGGAAGCAGCGCGAG
UUGGUCGCACGAGUUACUCAAGCUGGUAACAUCAACUAUGCAGACUCCGUGAAGGACCGAUUCACCAUCUCCAGAGAC
AAGGCCGAGAACGCGGUGUAUCUACAAAUGAACAGCCUCAAACCUGAGGACACGGCCGUCUACUACUGUAAUGGAGAU
CUUUUCGAUACGCCUUGGGGUCCAUCAAAUGACUACUGGGGCCAGGGGACCCAAGUCACCGUCUCCUCA
SEQ ID NO: 15: RNA encoding V_{H}H- 718
GGCCCGGGAGCGGCCGCUCAGGUGCAGCUGGUGGAGUCGGGGGGAGGUUUGGUGCAGCCUGGAGGGUCUCUGAGACUC
UCCUGUUUAGCCUCUGGAAUCACCUUCACUGUCUAUCGCAUGGCCUGGUACCGUCAGGCUCCGGGGAGGCAGCGCGAC
UUGGUCGCAGAAGUAGCUCCUGGUGGUGGAACGGUGGCUGCAAACUCCGUGAAGGGCCGAUUCACCAUCUCCAGAGAC
AGCGCCAAGAACACGGUGGAUCUGCAAAUGAACGACCUGAAACCUGACGAUACGGCCGUCUAUUAUUGUUAUGCACGU
AAUCUUUUCACGUCGGGGGAGUAUUGGGGCCAGGGGACCCAGGUCACCGUCUCCUCA
SEQ ID NO: 16: RNA encoding human IgG₁ Fc domain
GACAAAACUCACACAUGCCCACCGUGCCCAGCACCUGAACUCCUGGGGGGACCGUCAGUCUUCCUCUUCCCCCCAAAA
CCCAAGGACACCCUCAUGAUCUCCCGGACCCCUGAGGUCACAUGCGUGGUGGUGGACGUGAGCCACGAAGACCCUGAG
GUCAAGUUCAACUGGUACGUGGACGGCGUGGAGGUGCAUAAUGCCAAGACAAAGCCGCGGGAGGAGCAGUACAACAGC
ACGUACCGUGUGGUCAGCGUCCUCACCGUCCUGCACCAGGACUGGCUGAAUGGCAAGGAGUACAAGUGCAAGGUCUCC
AACAAAGCCCUCCCAGCCCCCAUCGAGAAAACCAUCUCCAAAGCCAAAGGGCAGCCCCGAGAACCACAGGUGUACACC
CUGCCCCCAUCCCGGGAGGAGAUGACCAAGAACCAGGUCAGCCUGACCUGCCUGGUCAAAGGCUUCUAUCCCAGCGAC
AUCGCCGUGGAGUGGGAGAGCAAUGGGCAGCCGGAGAACAACUACAAGACCACGCCUCCCGUGCUGGACUCCGACGGC
UCCUUCUUCCUCUACAGCAAGCUCACCGUGGACAAGAGCAGGUGGCAGCAGGGGAACGUCUUCUCAUGCUCCGUGAUG
CAUGAGGCUCUGCACAACCACUACACGCAGAAGAGCCUCUCCCUGUCUCCGGGUAAA
SEQ ID NO: 17: RNA encoding signal peptide
GAGUUCGGUCUUAGCUGGGUGUUUCUUGUCGCCCUGUUCAGAGGGGUACAAUGC
SEQ ID NO: 18: Full length VEEV-RNA polymerase-EVD68-IRS-Virus-like Particle (VLP)
This sequence is formatted as follows:
Noncoding regions-lower case
NSP ORF-UPPER CASE
GOI ORF-UPPERCASE BOLD
auaggcggcgcaugagagaagcccagaccaauuaccuacccaaaAUGGAGAAAGUUCACGUUGACAUCGAGGAAGACA
GCCCAUUCCUCAGAGCUUUGCAGCGGAGCUUCCCGCAGUUUGAGGUAGAAGCCAAGCAGGUCACUGAUAAUGACCAUG
CUAAUGCCAGAGCGUUUUCGCAUCUGGCUUCAAAACUGAUCGAAACGGAGGUGGACCCAUCCGACACGAUCCUUGACA
UUGGAAGUGCGCCCGCCCGCAGAAUGUAUUCUAAGCACAAGUAUCAUUGUAUCUGUCCGAUGAGAUGUGCGGAAGAUC
CGGACAGAUUGUAUAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGACGACGAGUCGUGUC
GCUACGAAGGGCAAGUCGCUGUUUACCAGGAUGUAUACGCGGUUGACGGACCGACAAGUCUCUAUCACCAAGCCAAUA
AGGGAGUUAGAGUCGCCUACUGGAUAGGCUUUGACACCACCCCUUUUAUGUUUAAGAACUUGGCUGGAGCAUAUCCAU
CAUACUCUACCAACUGGGCCGACGAAACCGUGUUAACGGCUCGUAACAUAGGCCUAUGCAGCUCUGACGUUAUGGAGC
GGUCACGUAGAGGGAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGUGGCAAGCAAAAUU
ACACAUGUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGAACGGGG
AGAGGGUCUCUUUUCCCGUGUGCACGUAUGUGCCAGCUACAUUGUGUGACCAAAUGACUGGCAUACUGGCAACAGAUG
UCAGUGCGGACGACGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAAUAUAAGGAAGAUC
AAGAAGAUGAAAGGCCACUAGGACUACGAGAUAGACAGUUAGUCAUGGGGUGUUGUUGGGCUUUUAGAAGGCACAAGA
UAACAUCUAUUUAUAAGCGCCCGGAUACCCAAACCAUCAUCAAAGUGAACAGCGAUUUCCACUCAUUCGUGCUGCCCA
GGAUAGGCAGUAACACAUUGGAGAUCGGGCUGAGAACAAGAAUCAGGAAAAUGUUAGAGGAGCACAAGGAGCCGUCAC
CUCUCAUUACCGCCGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGACUUGAUGUUACAAG
AGGCUGGGGCCGGCUCAGUGGAGACACCUCGUGGCUUGAUAAAGGUUACCAGCUACGAUGGCGAGGACAAGAUCGGCU
CUUACGCUGUGCUUUCUCCGCAGGCUGUACUCAAGAGUGAAAAAUUAUCUUGCAUCCACCCUCUCGCUGAACAAGUCA
UAGUGAUAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACUGUCAAGCCCAGCG
AGCACGACGGCGAAUACCUGUACGACAUCGACAGGAAACAGUGCGUCAAGAAAGAACUAGUCACUGGGCUAGGGCUCA
CAGGCGAGCUGGUGGAUCCUCCCUUCCAUGAAUUCGCCUACGAGAGUCUGAGAACACGACCAGCCGCUCCUUACCAAG
UACCAACCAUAGGGGUGUAUGGCGUGCCAGGAUCAGGCAAGUCUGGCAUCAUUAAAAGCGCAGUCACCAAAAAAGAUC
UAGUGGUGAGCGCCAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGACGAAGCUUUUGCUU
GUCAUGCAGGUACUCUCAGAGCGCUCAUAGCCAUUAUAAGACCUAAAAAGGCAGUGCUCUGCGGGGAUCCCAAACAGU
GCGGUUUUUUUAACAUGAUGUGCCUGAAAGUGCAUUUUAACCACGAGAUUUGCACACAAGUCUUCCACAAAAGCAUCU
CUCGCCGUUGCACUAAAUCUGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACGUCCUAC
UGACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACCGG
ACCCUACCGACGUCUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCAGAGAUAGUAUUGA
ACCAACUAUGCGUGAGGUUCUUUGGACUCGAUCUGGACUCCGGUCUAUUUUCUGCACCCACUGUUCCGUUAUCCAUUA
GGAAUAAUCACUGGGAUAACUCCCCGUCGCCUAACAUGUACGGGCUGAAUAAAGAAGUGGUCCGUCAGCUCUCUCGCA
GGUACCCACAACUGCCUCGGGCAGUUGCCACUGGAAGAGUCUAUGACAUGAACACUGGUACACUGCGCAAUUAUGAUC
CGCGCAUAAACCUAGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCCGUCCCAGGCAAAA
UGGUUGACUGGUUGUCAGACCGGCCUGAGGCUACCUUCAGAGCUCGGCUGGAUUUAGGCAUCCCAGGUGAUGUGCCCA
AAUAUGACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUGGUUACG
CUGACAGGGCCAGCGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAUCCUUACAAGCUUU
CAUCAACCUUGACCAACAUUUAUACAGGUUCCAGACUCCACGAAGCCGGAUGUGCACCCUCAUAUCAUGUGGUGCGAG
GGGAUAUUGCCACGGCCACCGAAGGAGUGAUUAUAAAUGCUGCUAACAGCAAAGGACAACCUGGCGGAGGGGUGUGCG
GAGCGCUGUAUAAGAAAUUCCCGGAAAGCUUCGAUUUACAGCCGAUCGAAGUAGGAAAAGCGCGACUGGUCAAAGGUG
CAGCUAAACAUAUCAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUGUUGUCCACCGGCA
UCUUUUCCGGGAACAAAGAUCGACUAACCCAAUCAUUGAACCAUUUGCUGACAGCUUUAGACACCACUGAUGCAGAUG
UAGCCAUAUACUGCAGGGACAAGAAAUGGGAAAUGACUCUCAAGGAAGCAGUGGCUAGGAGAGAAGCAGUGGAGGAGA
UAUGCAUAUCCGACGACUCUUCAGUGACAGAACCUGAUGCAGAGCUGGUGAGGGUGCAUCCGAAGAGUUCUUUGGCUG
GAAGGAAGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCGGAGAAA
GCAUGAGCAGUAUUAGGUCGAAAUGCCCCGUCGAAGAGUCGGAAGCCUCCACACCACCUAGCACGCUGCCUUGCUUGU
GCAUCCAUGCCAUGACUCCAGAAAGAGUACAGCGCCUAAAAGCCUCACGUCCAGAACAAAUUACUGUGUGCUCAUCCU
UUCCAUUGCCGAAGUAUAGAAUCACUGGUGUGCAGAAGAUCCAAUGCUCCCAGCCUAUAUUGUUCUCACCGAAAGUGC
CUGCGUAUAUUCAUCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACGCCUGAGCCGAUCA
UCAUCGAAGAGGAAGAAGAGGAUAGCAUAAGUUUGCUGUCAGAUGGCCCGACCCACCAGGUGCUGCAAGUCGAGGCAG
ACAUUCACGGGCCGCCCUCUGUAUCUAGCUCAUCCUGGUCCAUUCCUCAUGCAUCCGACUUUGAUGUGGACAGUUUAU
CCAUACUUGACACCCUGGAGGGAGCUAGCGUGACCAGCGGGGCAACGUCAGCCGAGACUAACUCUUACUUCGCAAAGA
GUAUGGAGUUUCUGGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGCGUGAAUAGGGUGA
UCACUAGAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGUUUGAUG
CGGGUGCAUACAUCUUUUCCUCCGACACCGGUCAAGGGCAUUUACAACAAAAAUCAGUAAGGCAAACGGUGCUAUCCG
AAGUGGUGUUGGAGAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUGU
UGAAAGAGAACUUUCCGACUGUGGCUUCUUACUGUAUUAUUCCAGAGUACGAUGCCUAUUUGGACAUGGUUGACGGAG
CUUCAUGCUGCUUAGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCCACAAAAAGAAAUU
GCAAUGUCACGCAAAUGAGAGAAUUGCCCGUAUUGGAUUCGGCGGCCUUUAAUGUGGAAUGCUUCAAGAAAUAUGCGU
GUAAUAAUGAAUAUUGGGAAACGUUUAAAGAAAACCCCAUCAGGCUUACUGAAGAAAACGUGGUAAAUUACAUUACCA
AAUUAAAAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGGAGAUUAAAUGCGG
UCCUGCUUCCGAACAUUCAUACACUGUUUGAUAUGUCGGCUGAAGACUUUGACGCUAUUAUAGCCGAGCACUUCCAGC
CUGGGGAUUGUGUUCUGGAAACUGACAUCGCGUCGUUUGAUAAAAGUGAGGACGACGCCAUGGCUCUGACCGCGUUAA
UGAUUCUGGAAGACUUAGGUGUGGACGCAGAGCUGUUGACGCUGAUUGAGGCGGCUUUCGGCGAAAUUUCAUCAAUAC
AUUUGCCCACUAAAACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCAUUCAUUGGAGAUG
ACAAUAUCGUGAAAGGAGUCAAAUCGGACAAAUUAAUGGCAGACAGGUGCGCCACCUGGUUGAAUAUGGAAGUCAAGA
UUAUAGAUGCUGUGGUGGGCGAGAAAGCGCCUUAUUUCUGUGGAGGGUUUAUUUUGUGUGACUCCGUGACCGGCACAG
CGUGCCGUGUGGCAGACCCCCUAAAAAGGCUGUUUAAGCUUGGCAAACCUCUGGCAGCAGACGAUGAACAUGAUGAUG
ACAGGAGAAGGGCAUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCuaaccugaauggacuacgacauagucuaguccgccaagAUGGG
UGCUCAGGUAACCAGGCAGCAAACCGGUACUCAUGAAAAUGCCAACAUAGCUACUAAUGGCUCCCAUAUUACGUACAA
UCAAAUCAAUUUCUACAAGGAUAGUUACGCUGCGUCCGCUUCUAAGCAGGACUUCAGUCAGGAUCCUAGCAAGUUUAC
GGAACCCGUAGUUGAAGGCCUUAAGGCAGGGGCACCUGUCCUUAAGUCACCGAGUGCGGAGGCUUGCGGUUACUCUGA
CCGAGUACUGCAGCUUAAGCUCGGGAACUCUGCCAUAGUUACGCAGGAAGCGGCAAACUAUUGCUGCGCGUACGGGGA
GUGGCCGAACUACCUGCCAGACCAUGAGGCGGUCGCUAUAGACAAGCCAACACAACCUGAGACAGCCACGGAUCGGUU
CUAUACUCUUAAAAGCGUAAAAUGGGAGACUGGCUCCACAGGAUGGUGGUGGAAGCUCCCAGAUGCCCUUAACAAUAU
CGGGAUGUUUGGCCAGAAUGUUCAACACCAUUACCUGUAUCGCAGUGGCUUCCUCAUUCACGUCCAGUGUAAUGCCAC
AAAGUUUCAUCAGGGGGCUCUCCUUGUGGUGGCGAUCCCAGAGCAUCAGAGGGGUGCACAUAAUACUAAUACUAGUCC
UGGUUUCGAUGAUAUAAUGAAAGGGGAGGAAGGAGGGACGUUUAAUCAUCCUUAUGUCCUGGAUGACGGGACCUCAUU
GGCGUGUGCGACGAUCUUCCCUCACCAGUGGAUUAAUCUCCGGACCAAUAACAGUGCGACUAUCGUACUUCCAUGGAU
GAACGCGGCUCCGAUGGAUUUUCCCCUGAGGCAUAAUCAGUGGACAUUGGCUAUUAUUCCGGUCGUACCCCUGGGUAC
UAGAACCACUAGCUCAAUGGUUCCCAUAACUGUAUCUAUUGCGCCAAUGUGCUGUGAAUUUAAUGGGCUCCGGCACGC
UAUCACACAAGGCGUUCCUACGUAUCUCUUGCCAGGCUCAGGUCAGUUCCUCACUACUGAUGACCAUAGCUCCGCACC
UGCCCUCCCCUGUUUUAACCCAACACCCGAGAUGCAUAUCCCAGGGCAAGUCCGAAACAUGCUUGAGGUUGUUCAGGU
AGAAUCUAUGAUGGAGAUCAAUAACACAGAGAGUGCGGUAGGGAUGGAGCGCCUUAAGGUUGACAUCUCCGCAUUGAC
CGAUGUUGACCAACUUUUGUUUAACAUUCCCCUGGAUAUACAGCUCGAUGGCCCCUUGCGGAACACGUUGGUCGGAAA
UAUCUCCAGGUACUACACUCAUUGGUCCGGCAGUCUCGAAAUGACAUUUAUGUUUUGCGGCAGUUUCAUGGCAGCGGG
CAAACUGAUCCUGUGUUAUACACCCCCAGGCGGUAGUUGUCCAACGACGCGAGAGACGGCGAUGCUCGGCACACAUAU
AGUGUGGGAUUUUGGCUUGCAAUCCUCAGUUACCCUCAUCAUACCGUGGAUAAGCGGCAGCCAUUAUAGAAUGUUCAA
CAAUGACGCUAAAAGUACGAACGCCAAUGUGGGAUAUGUGACCUGCUUUAUGCAGACGAAUCUCAUCGUACCUUCUGA
GUCCUCAGACACAUGCAGUUUGAUAGGUUUCAUAGCCGCAAAGGACGAUUUCAGUCUUAGACUUAUGCGGGACAGUCC
GGACAUUGGUCAACUGGAUCACCUUCAUGCUGCGGAGGCAGCAUAUCAGAUCGAAUCAAUAAUUAAAACUGCUACCGA
CACAGUCAAGUCCGAGAUAAACGCUGAACUGGGCGUCGUCCCGAGUCUUAAUGCAGUGGAAACCGGAGCCACUUCUAA
UACUGAGCCAGAAGAAGCAAUUCAAACUCGAACUGUGAUCAACCAACACGGUGUAAGCGAGACUUUGGUAGAAAAUUU
CCUCUCCAGAGCCGCCUUGGUAUCAAAAAGAAGUUUUGAGUAUAAAGACCACACGAGCUCUACAGCACGCGCAGACAA
GAACUUCUUUAAAUGGACGAUAAAUACCAGAAGUUUUGUACAGCUCCGCAGGAAAUUGGAGCUCUUCACAUACCUCCG
AUUUGACGCGGAAAUAACAAUUUUGACCACAGUUGCGGUUAAUGGUAGUGGAAAUAACACGUACGUAGGCUUGCCUGA
UCUGACACUGCAGGCCAUGUUUGUCCCUACUGGUGCACUCACUCCGGAGAAACAGGACUCCUUCCAUUGGCAGAGCGG
GUCAAAUGCGUCAGUGUUCUUCAAAAUCUCCGAUCCCCCCGCGAGGAUCACUAUUCCCUUUAUGUGUAUAAAUAGCGC
CUAUAGCGUUUUUUACGAUGGCUUUGCCGGCUUUGAAAAGAAUGGGUUGUACGGGAUUAAUCCGGCCGAUACGAUAGG
UAACCUGUGUGUACGCAUAGUUAACGAACACCAGCCAGUGGGUUUCACUGUAACCGUUCGAGUGUACAUGAAACCUAA
GCACAUCAAGGCUUGGGCACCAAGGCCACCGAGAACCCUCCCAUACAUGAGCAUUGCUAAUGCAAAUUAUAAGGGAAA
AGAGAGAGCACCGAACGCGUUGUCUGCAAUUAUCGGCAAUCGGGACUCAGUCAAGACUAUGCCACAUAAUAUAGUCAA
UACCUGACCCCUCUCCCUCCCCCCCCCCUAACGUUACUGGCCGAAGCCGCUUGGAAUAAGGCCGGUGUGCGUUUGUCU
AUAUGUUAUUUUCCACCAUAUUGCCGUCUUUUGGCAAUGUGAGGGCCCGGAAACCUGGCCCUGUCUUCUUGACGAGCA
UUCCUAGGGGUCUUUCCCCUCUCGCCAAAGGAAUGCAAGGUCUGUUGAAUGUCGUGAAGGAAGCAGUUCCUCUGGAAG
CUUCUUGAAGACAAACAACGUCUGUAGCGACCCUUUGCAGGCAGCGGAACCCCCCACCUGGCGACAGGUGCCUCUGCG
GCCAAAAGCCACGUGUAUAAGAUACACCUGCAAAGGCGGCACAACCCCAGUGCCACGUUGUGAGUUGGAUAGUUGUGG
AAAGAGUCAAAUGGCUCUCCUCAAGCGUAUUCAACAAGGGGCUGAAGGAUGCCCAGAAGGUACCCCAUUGUAUGGGAU
CUGAUCUGGGGCCUCGGUGCACAUGCUUUACAUGUGUUUAGUCGAGGUUAAAAAACGUCUAGGCCCCCCGAACCACGG
GGACGUGGUUUUCCUUUGAAAAACACGAUGAUAAUAUGGCCACAACCAUGGGUCCAGGCUUUGACUUUGCUCAGGCGA
UAAUGAAGAAAAACACGGUUAUCGCACGAACUGAAAAGGGCGAAUUUACCAUGCUCGGCGUGUACGAUCGAGUCGCGG
UUAUCCCGACACACGCUUCCGUGGGGGAAACCAUAUAUAUCAACGAUGUAGAAACCAAAGUCCUCGACGCGUGUGCAC
UGAGGGAUCUUACAGACACUAACCUGGAGAUCACAAUAGUGAAGCUGGAUCGAAAUCAGAAGUUCCGAGACAUCCGCC
AUUUUUUGCCUAGAUAUGAAGACGACUACAAUGAUGCUGUACUGUCCGUGCAUACUUCAAAGUUUCCUAACAUGUACA
UCCCUGUUGGCCAGGUCACUAAUUACGGUUUUCUUAACCUGGGUGGGACGCCUACUCAUAGGAUACUGAUGUACAAUU
UUCCUACUAGAGCUGGACAAUGUGGUGGCGUAGUGACUACCACCGGGAAAGUCAUUGGCAUACACGUAGGAGGUAACG
GGGCGCAGGGAUUCGCGGCCAUGCUUCUGCACAGCUAUUUCUCCGACACACAAGGUGAAAUAGUUUCAUCAGAGAAAU
CCGGUGUGUGCAUUAACGCUCCCGCGAAAACUAAACUUCAGCCCAGCGUGUUUCAUCAAGUAUUCGAAGGAAGCAAGG
AACCGGCUGUACUGAACCCCAAGGACCCCCGGCUUAAAACGGAUUUCGAAGAAGCGAUAUUUUCAAAAUAUACUGGUA
ACAAAAUCAUGCUGAUGGAUGAGUAUAUGGAAGAAGCUGUGGACCACUAUGUAGGGUGCCUGGAACCGCUCGACAUCU
CUGUGGACCCGAUCCCACUCGAGUCCGCUAUGUACGGCAUGGACGGCCUCGAGGCUUUGGACCUUACAACUAGCGCGG
GCUUUCCGUAUCUUUUGCAAGGUAAGAAAAAGCGCGACAUCUUUAACCGCCACACCAGGGAUACGAGCGAGAUGACAA
AAAUGCUUGAAAAAUAUGGGGUCGAUCUUCCUUUUGUCACUUUCGUGAAGGACGAAUUGAGAUCCCGAGAAAAGGUCG
AGAAAGGUAAGUCUCGCCUCAUUGAAGCCAGUUCACUUAAUGAUAGUGUUGCGAUGCGAGUUGCUUUUGGUAACCUUU
ACGCAACAUUUCAUAACAAUCCAGGCACGGCUACAGGAUCAGCAGUAGGUUGCGACCCAGACAUCUUUUGGUCAAAAA
UCCCCAUUCUGCUGGACGGUGAAAUUUUUGCCUUUGACUAUACCGGAUACGACGCAUCCUUGUCCCCUGUAUGGUUCG
CAUGUCUCAAAAAAGUCCUGAUAAAACUCGGUUACACUCACCAGACUAGUUUUAUAGACUAUCUGUGUCAUAGUGUUC
ACCUCUACAAAGAUAAAAAAUAUAUUGUGAACGGUGGUAUGCCGUCUGGUAGUUCCGGAACUUCCAUAUUUAACACAA
UGAUUAAUAAUAUCAUUAUAAGGACGCUUCUCAUCAGGGUCUACAAGGGUAUCGAUCUGGAUCAAUUCAAGAUGAUAG
CAUACGGCGACGACGUCAUUGCUUCUUACCCCCAUAAGAUUGAUCCAGGUCUGCUGGCGGAAGCCGGCAAGCAAUAUG
GACUGGUUAUGACACCCGCUGACAAAGGAACCAGUUUCAUCGACACGAAUUGGGAAAACGUGACGUUCCUGAAGCGAU
ACUUCAGAGCAGACGAUCAAUAUCCCUUUCUUAUCCAUCCCGUUAUGCCAAUGAAGGAGAUACACGAGUCAAUCCGAU
GGACAAAAGACCCACGGAACACACAAGAUCACGUCCGAUCACUCUGUUAUCUUGCCUGGCACAAUGGGGAGGAGGCGU
AUAAUGAGUUCUGCCGGAAGAUUCGAAGCGUACCAGUAGGCCGAGCACUGACUCUCCCUGCUUAUUCAAGUCUGCGGC
GGAAGUGGUUGGAUUCCUUCUAGuaaccgcggugucaaaaaccgcguggacgugguuaacaucccugcugggaggauc
agccguaauuauuauaauuggcuuggugcuggcuacuauuguggccauguacgugcugaccaaccagaaacauaauug
aauacagcagcaauuggcaagcugcuuacauagaacucgcggcgauuggcaugccgccuuaaaauuuuuauuuuauuu
uuucuuuucuuuuccgaaucggauuuuguuuuuaauauuucaaaaaaaaaaaaaaaaaaaaaaaaaaaaaaaaaaaaa
aaaaaaaa
SEQ ID NO: 19: 654-VEErep-EVD68-3CDdel
This sequence is formatted as follows:
Noncoding regions-lower case
NSP ORF-UPPER CASE
GOI ORF-UPPERCASE BOLD
auaggcggcgcaugagagaagcccagaccaauuaccuacccaaaAUGGAGAAAGUUCACGUUGACAUCGAGGAAGACA
GCCCAUUCCUCAGAGCUUUGCAGCGGAGCUUCCCGCAGUUUGAGGUAGAAGCCAAGCAGGUCACUGAUAAUGACCAUG
CUAAUGCCAGAGCGUUUUCGCAUCUGGCUUCAAAACUGAUCGAAACGGAGGUGGACCCAUCCGACACGAUCCUUGACA
UUGGAAGUGCGCCCGCCCGCAGAAUGUAUUCUAAGCACAAGUAUCAUUGUAUCUGUCCGAUGAGAUGUGCGGAAGAUC
CGGACAGAUUGUAUAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGACGACGAGUCGUGUC
GCUACGAAGGGCAAGUCGCUGUUUACCAGGAUGUAUACGCGGUUGACGGACCGACAAGUCUCUAUCACCAAGCCAAUA
AGGGAGUUAGAGUCGCCUACUGGAUAGGCUUUGACACCACCCCUUUUAUGUUUAAGAACUUGGCUGGAGCAUAUCCAU
CAUACUCUACCAACUGGGCCGACGAAACCGUGUUAACGGCUCGUAACAUAGGCCUAUGCAGCUCUGACGUUAUGGAGC
GGUCACGUAGAGGGAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGUGGCAAGCAAAAUU
ACACAUGUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGAACGGGG
AGAGGGUCUCUUUUCCCGUGUGCACGUAUGUGCCAGCUACAUUGUGUGACCAAAUGACUGGCAUACUGGCAACAGAUG
UCAGUGCGGACGACGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAAUAUAAGGAAGAUC
AAGAAGAUGAAAGGCCACUAGGACUACGAGAUAGACAGUUAGUCAUGGGGUGUUGUUGGGCUUUUAGAAGGCACAAGA
UAACAUCUAUUUAUAAGCGCCCGGAUACCCAAACCAUCAUCAAAGUGAACAGCGAUUUCCACUCAUUCGUGCUGCCCA
GGAUAGGCAGUAACACAUUGGAGAUCGGGCUGAGAACAAGAAUCAGGAAAAUGUUAGAGGAGCACAAGGAGCCGUCAC
CUCUCAUUACCGCCGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGACUUGAUGUUACAAG
AGGCUGGGGCCGGCUCAGUGGAGACACCUCGUGGCUUGAUAAAGGUUACCAGCUACGAUGGCGAGGACAAGAUCGGCU
CUUACGCUGUGCUUUCUCCGCAGGCUGUACUCAAGAGUGAAAAAUUAUCUUGCAUCCACCCUCUCGCUGAACAAGUCA
UAGUGAUAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACUGUCAAGCCCAGCG
AGCACGACGGCGAAUACCUGUACGACAUCGACAGGAAACAGUGCGUCAAGAAAGAACUAGUCACUGGGCUAGGGCUCA
CAGGCGAGCUGGUGGAUCCUCCCUUCCAUGAAUUCGCCUACGAGAGUCUGAGAACACGACCAGCCGCUCCUUACCAAG
UACCAACCAUAGGGGUGUAUGGCGUGCCAGGAUCAGGCAAGUCUGGCAUCAUUAAAAGCGCAGUCACCAAAAAAGAUC
UAGUGGUGAGCGCCAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGACGAAGCUUUUGCUU
GUCAUGCAGGUACUCUCAGAGCGCUCAUAGCCAUUAUAAGACCUAAAAAGGCAGUGCUCUGCGGGGAUCCCAAACAGU
GCGGUUUUUUUAACAUGAUGUGCCUGAAAGUGCAUUUUAACCACGAGAUUUGCACACAAGUCUUCCACAAAAGCAUCU
CUCGCCGUUGCACUAAAUCUGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACGUCCUAC
UGACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACCGG
ACCCUACCGACGUCUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCAGAGAUAGUAUUGA
ACCAACUAUGCGUGAGGUUCUUUGGACUCGAUCUGGACUCCGGUCUAUUUUCUGCACCCACUGUUCCGUUAUCCAUUA
GGAAUAAUCACUGGGAUAACUCCCCGUCGCCUAACAUGUACGGGCUGAAUAAAGAAGUGGUCCGUCAGCUCUCUCGCA
GGUACCCACAACUGCCUCGGGCAGUUGCCACUGGAAGAGUCUAUGACAUGAACACUGGUACACUGCGCAAUUAUGAUC
CGCGCAUAAACCUAGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCCGUCCCAGGCAAAA
UGGUUGACUGGUUGUCAGACCGGCCUGAGGCUACCUUCAGAGCUCGGCUGGAUUUAGGCAUCCCAGGUGAUGUGCCCA
AAUAUGACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUGGUUACG
CUGACAGGGCCAGCGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAUCCUUACAAGCUUU
CAUCAACCUUGACCAACAUUUAUACAGGUUCCAGACUCCACGAAGCCGGAUGUGCACCCUCAUAUCAUGUGGUGCGAG
GGGAUAUUGCCACGGCCACCGAAGGAGUGAUUAUAAAUGCUGCUAACAGCAAAGGACAACCUGGCGGAGGGGUGUGCG
GAGCGCUGUAUAAGAAAUUCCCGGAAAGCUUCGAUUUACAGCCGAUCGAAGUAGGAAAAGCGCGACUGGUCAAAGGUG
CAGCUAAACAUAUCAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUGUUGUCCACCGGCA
UCUUUUCCGGGAACAAAGAUCGACUAACCCAAUCAUUGAACCAUUUGCUGACAGCUUUAGACACCACUGAUGCAGAUG
UAGCCAUAUACUGCAGGGACAAGAAAUGGGAAAUGACUCUCAAGGAAGCAGUGGCUAGGAGAGAAGCAGUGGAGGAGA
UAUGCAUAUCCGACGACUCUUCAGUGACAGAACCUGAUGCAGAGCUGGUGAGGGUGCAUCCGAAGAGUUCUUUGGCUG
GAAGGAAGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCGGAGAAA
GCAUGAGCAGUAUUAGGUCGAAAUGCCCCGUCGAAGAGUCGGAAGCCUCCACACCACCUAGCACGCUGCCUUGCUUGU
GCAUCCAUGCCAUGACUCCAGAAAGAGUACAGCGCCUAAAAGCCUCACGUCCAGAACAAAUUACUGUGUGCUCAUCCU
UUCCAUUGCCGAAGUAUAGAAUCACUGGUGUGCAGAAGAUCCAAUGCUCCCAGCCUAUAUUGUUCUCACCGAAAGUGC
CUGCGUAUAUUCAUCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACGCCUGAGCCGAUCA
UCAUCGAAGAGGAAGAAGAGGAUAGCAUAAGUUUGCUGUCAGAUGGCCCGACCCACCAGGUGCUGCAAGUCGAGGCAG
ACAUUCACGGGCCGCCCUCUGUAUCUAGCUCAUCCUGGUCCAUUCCUCAUGCAUCCGACUUUGAUGUGGACAGUUUAU
CCAUACUUGACACCCUGGAGGGAGCUAGCGUGACCAGCGGGGCAACGUCAGCCGAGACUAACUCUUACUUCGCAAAGA
GUAUGGAGUUUCUGGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGCGUGAAUAGGGUGA
UCACUAGAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGUUUGAUG
CGGGUGCAUACAUCUUUUCCUCCGACACCGGUCAAGGGCAUUUACAACAAAAAUCAGUAAGGCAAACGGUGCUAUCCG
AAGUGGUGUUGGAGAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUGU
UGAAAGAGAACUUUCCGACUGUGGCUUCUUACUGUAUUAUUCCAGAGUACGAUGCCUAUUUGGACAUGGUUGACGGAG
CUUCAUGCUGCUUAGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCCACAAAAAGAAAUU
GCAAUGUCACGCAAAUGAGAGAAUUGCCCGUAUUGGAUUCGGCGGCCUUUAAUGUGGAAUGCUUCAAGAAAUAUGCGU
GUAAUAAUGAAUAUUGGGAAACGUUUAAAGAAAACCCCAUCAGGCUUACUGAAGAAAACGUGGUAAAUUACAUUACCA
AAUUAAAAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGGAGAUUAAAUGCGG
UCCUGCUUCCGAACAUUCAUACACUGUUUGAUAUGUCGGCUGAAGACUUUGACGCUAUUAUAGCCGAGCACUUCCAGC
CUGGGGAUUGUGUUCUGGAAACUGACAUCGCGUCGUUUGAUAAAAGUGAGGACGACGCCAUGGCUCUGACCGCGUUAA
UGAUUCUGGAAGACUUAGGUGUGGACGCAGAGCUGUUGACGCUGAUUGAGGCGGCUUUCGGCGAAAUUUCAUCAAUAC
AUUUGCCCACUAAAACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCAUUCAUUGGAGAUG
ACAAUAUCGUGAAAGGAGUCAAAUCGGACAAAUUAAUGGCAGACAGGUGCGCCACCUGGUUGAAUAUGGAAGUCAAGA
UUAUAGAUGCUGUGGUGGGCGAGAAAGCGCCUUAUUUCUGUGGAGGGUUUAUUUUGUGUGACUCCGUGACCGGCACAG
CGUGCCGUGUGGCAGACCCCCUAAAAAGGCUGUUUAAGCUUGGCAAACCUCUGGCAGCAGACGAUGAACAUGAUGAUG
ACAGGAGAAGGGCAUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCuaaccugaauggacuacgacauagucuaguccgccaagAUGGG
UGCUCAGGUAACCAGGCAGCAAACCGGUACUCAUGAAAAUGCCAACAUAGCUACUAAUGGCUCCCAUAUUACGUACAA
UCAAAUCAAUUUCUACAAGGAUAGUUACGCUGCGUCCGCUUCUAAGCAGGACUUCAGUCAGGAUCCUAGCAAGUUUAC
GGAACCCGUAGUUGAAGGCCUUAAGGCAGGGGCACCUGUCCUUAAGUCACCGAGUGCGGAGGCUUGCGGUUACUCUGA
CCGAGUACUGCAGCUUAAGCUCGGGAACUCUGCCAUAGUUACGCAGGAAGCGGCAAACUAUUGCUGCGCGUACGGGGA
GUGGCCGAACUACCUGCCAGACCAUGAGGCGGUCGCUAUAGACAAGCCAACACAACCUGAGACAGCCACGGAUCGGUU
CUAUACUCUUAAAAGCGUAAAAUGGGAGACUGGCUCCACAGGAUGGUGGUGGAAGCUCCCAGAUGCCCUUAACAAUAU
CGGGAUGUUUGGCCAGAAUGUUCAACACCAUUACCUGUAUCGCAGUGGCUUCCUCAUUCACGUCCAGUGUAAUGCCAC
AAAGUUUCAUCAGGGGGCUCUCCUUGUGGUGGCGAUCCCAGAGCAUCAGAGGGGUGCACAUAAUACUAAUACUAGUCC
UGGUUUCGAUGAUAUAAUGAAAGGGGAGGAAGGAGGGACGUUUAAUCAUCCUUAUGUCCUGGAUGACGGGACCUCAUU
GGCGUGUGCGACGAUCUUCCCUCACCAGUGGAUUAAUCUCCGGACCAAUAACAGUGCGACUAUCGUACUUCCAUGGAU
GAACGCGGCUCCGAUGGAUUUUCCCCUGAGGCAUAAUCAGUGGACAUUGGCUAUUAUUCCGGUCGUACCCCUGGGUAC
UAGAACCACUAGCUCAAUGGUUCCCAUAACUGUAUCUAUUGCGCCAAUGUGCUGUGAAUUUAAUGGGCUCCGGCACGC
UAUCACACAAGGCGUUCCUACGUAUCUCUUGCCAGGCUCAGGUCAGUUCCUCACUACUGAUGACCAUAGCUCCGCACC
UGCCCUCCCCUGUUUUAACCCAACACCCGAGAUGCAUAUCCCAGGGCAAGUCCGAAACAUGCUUGAGGUUGUUCAGGU
AGAAUCUAUGAUGGAGAUCAAUAACACAGAGAGUGCGGUAGGGAUGGAGCGCCUUAAGGUUGACAUCUCCGCAUUGAC
CGAUGUUGACCAACUUUUGUUUAACAUUCCCCUGGAUAUACAGCUCGAUGGCCCCUUGCGGAACACGUUGGUCGGAAA
UAUCUCCAGGUACUACACUCAUUGGUCCGGCAGUCUCGAAAUGACAUUUAUGUUUUGCGGCAGUUUCAUGGCAGCGGG
CAAACUGAUCCUGUGUUAUACACCCCCAGGCGGUAGUUGUCCAACGACGCGAGAGACGGCGAUGCUCGGCACACAUAU
AGUGUGGGAUUUUGGCUUGCAAUCCUCAGUUACCCUCAUCAUACCGUGGAUAAGCGGCAGCCAUUAUAGAAUGUUCAA
CAAUGACGCUAAAAGUACGAACGCCAAUGUGGGAUAUGUGACCUGCUUUAUGCAGACGAAUCUCAUCGUACCUUCUGA
GUCCUCAGACACAUGCAGUUUGAUAGGUUUCAUAGCCGCAAAGGACGAUUUCAGUCUUAGACUUAUGCGGGACAGUCC
GGACAUUGGUCAACUGGAUCACCUUCAUGCUGCGGAGGCAGCAUAUCAGAUCGAAUCAAUAAUUAAAACUGCUACCGA
CACAGUCAAGUCCGAGAUAAACGCUGAACUGGGCGUCGUCCCGAGUCUUAAUGCAGUGGAAACCGGAGCCACUUCUAA
UACUGAGCCAGAAGAAGCAAUUCAAACUCGAACUGUGAUCAACCAACACGGUGUAAGCGAGACUUUGGUAGAAAAUUU
CCUCUCCAGAGCCGCCUUGGUAUCAAAAAGAAGUUUUGAGUAUAAAGACCACACGAGCUCUACAGCACGCGCAGACAA
GAACUUCUUUAAAUGGACGAUAAAUACCAGAAGUUUUGUACAGCUCCGCAGGAAAUUGGAGCUCUUCACAUACCUCCG
AUUUGACGCGGAAAUAACAAUUUUGACCACAGUUGCGGUUAAUGGUAGUGGAAAUAACACGUACGUAGGCUUGCCUGA
UCUGACACUGCAGGCCAUGUUUGUCCCUACUGGUGCACUCACUCCGGAGAAACAGGACUCCUUCCAUUGGCAGAGCGG
GUCAAAUGCGUCAGUGUUCUUCAAAAUCUCCGAUCCCCCCGCGAGGAUCACUAUUCCCUUUAUGUGUAUAAAUAGCGC
CUAUAGCGUUUUUUACGAUGGCUUUGCCGGCUUUGAAAAGAAUGGGUUGUACGGGAUUAAUCCGGCCGAUACGAUAGG
UAACCUGUGUGUACGCAUAGUUAACGAACACCAGCCAGUGGGUUUCACUGUAACCGUUCGAGUGUACAUGAAACCUAA
GCACAUCAAGGCUUGGGCACCAAGGCCACCGAGAACCCUCCCAUACAUGAGCAUUGCUAAUGCAAAUUAUAAGGGAAA
AGAGAGAGCACCGAACGCGUUGUCUGCAAUUAUCGGCAAUCGGGACUCAGUCAAGACUAUGCCACAUAAUAUAGUCAA
UACCugauaaccgcggugucaaaaaccgcguggacgugguuaacaucccugcugggaggaucagccguaauuauuaua
auuggcuuggugcuggcuacuauuguggccauguacgugcugaccaaccagaaacauaauugaauacagcagcaauug
gcaagcugcuuacauagaacucgcggcgauuggcaugccgccuuaaaauuuuuauuuuauuuuuucuuuucuuuuccg
aaucggauuuuguuuuuaauauuucaaaaaaaaaaaaaaaaaaaaaaaaaaaaaaaaaaaaaaaaaaaaa
SEQ ID NO: 20: 637-VEEV-EVD68-T2A-VLP
This sequence is formatted as follows:
Noncoding regions-lower case
NSP ORF-UPPER CASE
GOI ORF-UPPERCASE BOLD
auaggcggcgcaugagagaagcccagaccaauuaccuacccaaaAUGGAGAAAGUUCACGUUGACAUCGAGGAAGACA
GCCCAUUCCUCAGAGCUUUGCAGCGGAGCUUCCCGCAGUUUGAGGUAGAAGCCAAGCAGGUCACUGAUAAUGACCAUG
CUAAUGCCAGAGCGUUUUCGCAUCUGGCUUCAAAACUGAUCGAAACGGAGGUGGACCCAUCCGACACGAUCCUUGACA
UUGGAAGUGCGCCCGCCCGCAGAAUGUAUUCUAAGCACAAGUAUCAUUGUAUCUGUCCGAUGAGAUGUGCGGAAGAUC
CGGACAGAUUGUAUAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGACGACGAGUCGUGUC
GCUACGAAGGGCAAGUCGCUGUUUACCAGGAUGUAUACGCGGUUGACGGACCGACAAGUCUCUAUCACCAAGCCAAUA
AGGGAGUUAGAGUCGCCUACUGGAUAGGCUUUGACACCACCCCUUUUAUGUUUAAGAACUUGGCUGGAGCAUAUCCAU
CAUACUCUACCAACUGGGCCGACGAAACCGUGUUAACGGCUCGUAACAUAGGCCUAUGCAGCUCUGACGUUAUGGAGC
GGUCACGUAGAGGGAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGUGGCAAGCAAAAUU
ACACAUGUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGAACGGGG
AGAGGGUCUCUUUUCCCGUGUGCACGUAUGUGCCAGCUACAUUGUGUGACCAAAUGACUGGCAUACUGGCAACAGAUG
UCAGUGCGGACGACGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAAUAUAAGGAAGAUC
AAGAAGAUGAAAGGCCACUAGGACUACGAGAUAGACAGUUAGUCAUGGGGUGUUGUUGGGCUUUUAGAAGGCACAAGA
UAACAUCUAUUUAUAAGCGCCCGGAUACCCAAACCAUCAUCAAAGUGAACAGCGAUUUCCACUCAUUCGUGCUGCCCA
GGAUAGGCAGUAACACAUUGGAGAUCGGGCUGAGAACAAGAAUCAGGAAAAUGUUAGAGGAGCACAAGGAGCCGUCAC
CUCUCAUUACCGCCGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGACUUGAUGUUACAAG
AGGCUGGGGCCGGCUCAGUGGAGACACCUCGUGGCUUGAUAAAGGUUACCAGCUACGAUGGCGAGGACAAGAUCGGCU
CUUACGCUGUGCUUUCUCCGCAGGCUGUACUCAAGAGUGAAAAAUUAUCUUGCAUCCACCCUCUCGCUGAACAAGUCA
UAGUGAUAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACUGUCAAGCCCAGCG
AGCACGACGGCGAAUACCUGUACGACAUCGACAGGAAACAGUGCGUCAAGAAAGAACUAGUCACUGGGCUAGGGCUCA
CAGGCGAGCUGGUGGAUCCUCCCUUCCAUGAAUUCGCCUACGAGAGUCUGAGAACACGACCAGCCGCUCCUUACCAAG
UACCAACCAUAGGGGUGUAUGGCGUGCCAGGAUCAGGCAAGUCUGGCAUCAUUAAAAGCGCAGUCACCAAAAAAGAUC
UAGUGGUGAGCGCCAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGACGAAGCUUUUGCUU
GUCAUGCAGGUACUCUCAGAGCGCUCAUAGCCAUUAUAAGACCUAAAAAGGCAGUGCUCUGCGGGGAUCCCAAACAGU
GCGGUUUUUUUAACAUGAUGUGCCUGAAAGUGCAUUUUAACCACGAGAUUUGCACACAAGUCUUCCACAAAAGCAUCU
CUCGCCGUUGCACUAAAUCUGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACGUCCUAC
UGACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACCGG
ACCCUACCGACGUCUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCAGAGAUAGUAUUGA
ACCAACUAUGCGUGAGGUUCUUUGGACUCGAUCUGGACUCCGGUCUAUUUUCUGCACCCACUGUUCCGUUAUCCAUUA
GGAAUAAUCACUGGGAUAACUCCCCGUCGCCUAACAUGUACGGGCUGAAUAAAGAAGUGGUCCGUCAGCUCUCUCGCA
GGUACCCACAACUGCCUCGGGCAGUUGCCACUGGAAGAGUCUAUGACAUGAACACUGGUACACUGCGCAAUUAUGAUC
CGCGCAUAAACCUAGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCCGUCCCAGGCAAAA
UGGUUGACUGGUUGUCAGACCGGCCUGAGGCUACCUUCAGAGCUCGGCUGGAUUUAGGCAUCCCAGGUGAUGUGCCCA
AAUAUGACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUGGUUACG
CUGACAGGGCCAGCGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAUCCUUACAAGCUUU
CAUCAACCUUGACCAACAUUUAUACAGGUUCCAGACUCCACGAAGCCGGAUGUGCACCCUCAUAUCAUGUGGUGCGAG
GGGAUAUUGCCACGGCCACCGAAGGAGUGAUUAUAAAUGCUGCUAACAGCAAAGGACAACCUGGCGGAGGGGUGUGCG
GAGCGCUGUAUAAGAAAUUCCCGGAAAGCUUCGAUUUACAGCCGAUCGAAGUAGGAAAAGCGCGACUGGUCAAAGGUG
CAGCUAAACAUAUCAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUGUUGUCCACCGGCA
UCUUUUCCGGGAACAAAGAUCGACUAACCCAAUCAUUGAACCAUUUGCUGACAGCUUUAGACACCACUGAUGCAGAUG
UAGCCAUAUACUGCAGGGACAAGAAAUGGGAAAUGACUCUCAAGGAAGCAGUGGCUAGGAGAGAAGCAGUGGAGGAGA
UAUGCAUAUCCGACGACUCUUCAGUGACAGAACCUGAUGCAGAGCUGGUGAGGGUGCAUCCGAAGAGUUCUUUGGCUG
GAAGGAAGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCGGAGAAA
GCAUGAGCAGUAUUAGGUCGAAAUGCCCCGUCGAAGAGUCGGAAGCCUCCACACCACCUAGCACGCUGCCUUGCUUGU
GCAUCCAUGCCAUGACUCCAGAAAGAGUACAGCGCCUAAAAGCCUCACGUCCAGAACAAAUUACUGUGUGCUCAUCCU
UUCCAUUGCCGAAGUAUAGAAUCACUGGUGUGCAGAAGAUCCAAUGCUCCCAGCCUAUAUUGUUCUCACCGAAAGUGC
CUGCGUAUAUUCAUCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACGCCUGAGCCGAUCA
UCAUCGAAGAGGAAGAAGAGGAUAGCAUAAGUUUGCUGUCAGAUGGCCCGACCCACCAGGUGCUGCAAGUCGAGGCAG
ACAUUCACGGGCCGCCCUCUGUAUCUAGCUCAUCCUGGUCCAUUCCUCAUGCAUCCGACUUUGAUGUGGACAGUUUAU
CCAUACUUGACACCCUGGAGGGAGCUAGCGUGACCAGCGGGGCAACGUCAGCCGAGACUAACUCUUACUUCGCAAAGA
GUAUGGAGUUUCUGGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGCGUGAAUAGGGUGA
UCACUAGAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGUUUGAUG
CGGGUGCAUACAUCUUUUCCUCCGACACCGGUCAAGGGCAUUUACAACAAAAAUCAGUAAGGCAAACGGUGCUAUCCG
AAGUGGUGUUGGAGAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUGU
UGAAAGAGAACUUUCCGACUGUGGCUUCUUACUGUAUUAUUCCAGAGUACGAUGCCUAUUUGGACAUGGUUGACGGAG
CUUCAUGCUGCUUAGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCCACAAAAAGAAAUU
GCAAUGUCACGCAAAUGAGAGAAUUGCCCGUAUUGGAUUCGGCGGCCUUUAAUGUGGAAUGCUUCAAGAAAUAUGCGU
GUAAUAAUGAAUAUUGGGAAACGUUUAAAGAAAACCCCAUCAGGCUUACUGAAGAAAACGUGGUAAAUUACAUUACCA
AAUUAAAAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGGAGAUUAAAUGCGG
UCCUGCUUCCGAACAUUCAUACACUGUUUGAUAUGUCGGCUGAAGACUUUGACGCUAUUAUAGCCGAGCACUUCCAGC
CUGGGGAUUGUGUUCUGGAAACUGACAUCGCGUCGUUUGAUAAAAGUGAGGACGACGCCAUGGCUCUGACCGCGUUAA
UGAUUCUGGAAGACUUAGGUGUGGACGCAGAGCUGUUGACGCUGAUUGAGGCGGCUUUCGGCGAAAUUUCAUCAAUAC
AUUUGCCCACUAAAACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCAUUCAUUGGAGAUG
ACAAUAUCGUGAAAGGAGUCAAAUCGGACAAAUUAAUGGCAGACAGGUGCGCCACCUGGUUGAAUAUGGAAGUCAAGA
UUAUAGAUGCUGUGGUGGGCGAGAAAGCGCCUUAUUUCUGUGGAGGGUUUAUUUUGUGUGACUCCGUGACCGGCACAG
CGUGCCGUGUGGCAGACCCCCUAAAAAGGCUGUUUAAGCUUGGCAAACCUCUGGCAGCAGACGAUGAACAUGAUGAUG
ACAGGAGAAGGGCAUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCuaaccugaauggacuacgacauagucuaguccgccaagAUGGG
UGCUCAGGUAACCAGGCAGCAAACCGGUACUCAUGAAAAUGCCAACAUAGCUACUAAUGGCUCCCAUAUUACGUACAA
UCAAAUCAAUUUCUACAAGGAUAGUUACGCUGCGUCCGCUUCUAAGCAGGACUUCAGUCAGGAUCCUAGCAAGUUUAC
GGAACCCGUAGUUGAAGGCCUUAAGGCAGGGGCACCUGUCCUUAAGUCACCGAGUGCGGAGGCUUGCGGUUACUCUGA
CCGAGUACUGCAGCUUAAGCUCGGGAACUCUGCCAUAGUUACGCAGGAAGCGGCAAACUAUUGCUGCGCGUACGGGGA
GUGGCCGAACUACCUGCCAGACCAUGAGGCGGUCGCUAUAGACAAGCCAACACAACCUGAGACAGCCACGGAUCGGUU
CUAUACUCUUAAAAGCGUAAAAUGGGAGACUGGCUCCACAGGAUGGUGGUGGAAGCUCCCAGAUGCCCUUAACAAUAU
CGGGAUGUUUGGCCAGAAUGUUCAACACCAUUACCUGUAUCGCAGUGGCUUCCUCAUUCACGUCCAGUGUAAUGCCAC
AAAGUUUCAUCAGGGGGCUCUCCUUGUGGUGGCGAUCCCAGAGCAUCAGAGGGGUGCACAUAAUACUAAUACUAGUCC
UGGUUUCGAUGAUAUAAUGAAAGGGGAGGAAGGAGGGACGUUUAAUCAUCCUUAUGUCCUGGAUGACGGGACCUCAUU
GGCGUGUGCGACGAUCUUCCCUCACCAGUGGAUUAAUCUCCGGACCAAUAACAGUGCGACUAUCGUACUUCCAUGGAU
GAACGCGGCUCCGAUGGAUUUUCCCCUGAGGCAUAAUCAGUGGACAUUGGCUAUUAUUCCGGUCGUACCCCUGGGUAC
UAGAACCACUAGCUCAAUGGUUCCCAUAACUGUAUCUAUUGCGCCAAUGUGCUGUGAAUUUAAUGGGCUCCGGCACGC
UAUCACACAAGGCGUUCCUACGUAUCUCUUGCCAGGCUCAGGUCAGUUCCUCACUACUGAUGACCAUAGCUCCGCACC
UGCCCUCCCCUGUUUUAACCCAACACCCGAGAUGCAUAUCCCAGGGCAAGUCCGAAACAUGCUUGAGGUUGUUCAGGU
AGAAUCUAUGAUGGAGAUCAAUAACACAGAGAGUGCGGUAGGGAUGGAGCGCCUUAAGGUUGACAUCUCCGCAUUGAC
CGAUGUUGACCAACUUUUGUUUAACAUUCCCCUGGAUAUACAGCUCGAUGGCCCCUUGCGGAACACGUUGGUCGGAAA
UAUCUCCAGGUACUACACUCAUUGGUCCGGCAGUCUCGAAAUGACAUUUAUGUUUUGCGGCAGUUUCAUGGCAGCGGG
CAAACUGAUCCUGUGUUAUACACCCCCAGGCGGUAGUUGUCCAACGACGCGAGAGACGGCGAUGCUCGGCACACAUAU
AGUGUGGGAUUUUGGCUUGCAAUCCUCAGUUACCCUCAUCAUACCGUGGAUAAGCGGCAGCCAUUAUAGAAUGUUCAA
CAAUGACGCUAAAAGUACGAACGCCAAUGUGGGAUAUGUGACCUGCUUUAUGCAGACGAAUCUCAUCGUACCUUCUGA
GUCCUCAGACACAUGCAGUUUGAUAGGUUUCAUAGCCGCAAAGGACGAUUUCAGUCUUAGACUUAUGCGGGACAGUCC
GGACAUUGGUCAACUGGAUCACCUUCAUGCUGCGGAGGCAGCAUAUCAGAUCGAAUCAAUAAUUAAAACUGCUACCGA
CACAGUCAAGUCCGAGAUAAACGCUGAACUGGGCGUCGUCCCGAGUCUUAAUGCAGUGGAAACCGGAGCCACUUCUAA
UACUGAGCCAGAAGAAGCAAUUCAAACUCGAACUGUGAUCAACCAACACGGUGUAAGCGAGACUUUGGUAGAAAAUUU
CCUCUCCAGAGCCGCCUUGGUAUCAAAAAGAAGUUUUGAGUAUAAAGACCACACGAGCUCUACAGCACGCGCAGACAA
GAACUUCUUUAAAUGGACGAUAAAUACCAGAAGUUUUGUACAGCUCCGCAGGAAAUUGGAGCUCUUCACAUACCUCCG
AUUUGACGCGGAAAUAACAAUUUUGACCACAGUUGCGGUUAAUGGUAGUGGAAAUAACACGUACGUAGGCUUGCCUGA
UCUGACACUGCAGGCCAUGUUUGUCCCUACUGGUGCACUCACUCCGGAGAAACAGGACUCCUUCCAUUGGCAGAGCGG
GUCAAAUGCGUCAGUGUUCUUCAAAAUCUCCGAUCCCCCCGCGAGGAUCACUAUUCCCUUUAUGUGUAUAAAUAGCGC
CUAUAGCGUUUUUUACGAUGGCUUUGCCGGCUUUGAAAAGAAUGGGUUGUACGGGAUUAAUCCGGCCGAUACGAUAGG
UAACCUGUGUGUACGCAUAGUUAACGAACACCAGCCAGUGGGUUUCACUGUAACCGUUCGAGUGUACAUGAAACCUAA
GCACAUCAAGGCUUGGGCACCAAGGCCACCGAGAACCCUCCCAUACAUGAGCAUUGCUAAUGCAAAUUAUAAGGGAAA
AGAGAGAGCACCGAACGCGUUGUCUGCAAUUAUCGGCAAUCGGGACUCAGUCAAGACUAUGCCACAUAAUAUAGUCAA
UACCCGCCGGAAGCGGGGUAGCGGAGAGGGGCGCGGGUCACUGUUGACGUGCGGGGACGUGGAAGAAAAUCCGGGGCC
UGGUCCAGGCUUUGACUUUGCUCAGGCGAUAAUGAAGAAAAACACGGUUAUCGCACGAACUGAAAAGGGCGAAUUUAC
CAUGCUCGGCGUGUACGAUCGAGUCGCGGUUAUCCCGACACACGCUUCCGUGGGGGAAACCAUAUAUAUCAACGAUGU
AGAAACCAAAGUCCUCGACGCGUGUGCACUGAGGGAUCUUACAGACACUAACCUGGAGAUCACAAUAGUGAAGCUGGA
UCGAAAUCAGAAGUUCCGAGACAUCCGCCAUUUUUUGCCUAGAUAUGAAGACGACUACAAUGAUGCUGUACUGUCCGU
GCAUACUUCAAAGUUUCCUAACAUGUACAUCCCUGUUGGCCAGGUCACUAAUUACGGUUUUCUUAACCUGGGUGGGAC
GCCUACUCAUAGGAUACUGAUGUACAAUUUUCCUACUAGAGCUGGACAAUGUGGUGGCGUAGUGACUACCACCGGGAA
AGUCAUUGGCAUACACGUAGGAGGUAACGGGGCGCAGGGAUUCGCGGCCAUGCUUCUGCACAGCUAUUUCUCCGACAC
ACAAGGUGAAAUAGUUUCAUCAGAGAAAUCCGGUGUGUGCAUUAACGCUCCCGCGAAAACUAAACUUCAGCCCAGCGU
GUUUCAUCAAGUAUUCGAAGGAAGCAAGGAACCGGCUGUACUGAACCCCAAGGACCCCCGGCUUAAAACGGAUUUCGA
AGAAGCGAUAUUUUCAAAAUAUACUGGUAACAAAAUCAUGCUGAUGGAUGAGUAUAUGGAAGAAGCUGUGGACCACUA
UGUAGGGUGCCUGGAACCGCUCGACAUCUCUGUGGACCCGAUCCCACUCGAGUCCGCUAUGUACGGCAUGGACGGCCU
CGAGGCUUUGGACCUUACAACUAGCGCGGGCUUUCCGUAUCUUUUGCAAGGUAAGAAAAAGCGCGACAUCUUUAACCG
CCACACCAGGGAUACGAGCGAGAUGACAAAAAUGCUUGAAAAAUAUGGGGUCGAUCUUCCUUUUGUCACUUUCGUGAA
GGACGAAUUGAGAUCCCGAGAAAAGGUCGAGAAAGGUAAGUCUCGCCUCAUUGAAGCCAGUUCACUUAAUGAUAGUGU
UGCGAUGCGAGUUGCUUUUGGUAACCUUUACGCAACAUUUCAUAACAAUCCAGGCACGGCUACAGGAUCAGCAGUAGG
UUGCGACCCAGACAUCUUUUGGUCAAAAAUCCCCAUUCUGCUGGACGGUGAAAUUUUUGCCUUUGACUAUACCGGAUA
CGACGCAUCCUUGUCCCCUGUAUGGUUCGCAUGUCUCAAAAAAGUCCUGAUAAAACUCGGUUACACUCACCAGACUAG
UUUUAUAGACUAUCUGUGUCAUAGUGUUCACCUCUACAAAGAUAAAAAAUAUAUUGUGAACGGUGGUAUGCCGUCUGG
UAGUUCCGGAACUUCCAUAUUUAACACAAUGAUUAAUAAUAUCAUUAUAAGGACGCUUCUCAUCAGGGUCUACAAGGG
UAUCGAUCUGGAUCAAUUCAAGAUGAUAGCAUACGGCGACGACGUCAUUGCUUCUUACCCCCAUAAGAUUGAUCCAGG
UCUGCUGGCGGAAGCCGGCAAGCAAUAUGGACUGGUUAUGACACCCGCUGACAAAGGAACCAGUUUCAUCGACACGAA
UUGGGAAAACGUGACGUUCCUGAAGCGAUACUUCAGAGCAGACGAUCAAUAUCCCUUUCUUAUCCAUCCCGUUAUGCC
AAUGAAGGAGAUACACGAGUCAAUCCGAUGGACAAAAGACCCACGGAACACACAAGAUCACGUCCGAUCACUCUGUUA
UCUUGCCUGGCACAAUGGGGAGGAGGCGUAUAAUGAGUUCUGCCGGAAGAUUCGAAGCGUACCAGUAGGCCGAGCACU
GACUCUCCCUGCUUAUUCAAGUCUGCGGCGGAAGUGGUUGGAUUCCUUCUAGuaaccgcggugucaaaaaccgcgugg
acgugguuaacaucccugcugggaggaucagccguaauuauuauaauuggcuuggugcuggcuacuauuguggccaug
uacgugcugaccaaccagaaacauaauugaauacagcagcaauuggcaagcugcuuacauagaacucgcggcgauugg
caugccgccuuaaaauuuuuauuuuauuuuuucuuuucuuuuccgaaucggauuuuguuuuuaauauuucaaaaaaaa
aaaaaaaaaaaaaaaaaaaaaaaaaaaaaaaaaaaaa
SEQ ID NO: 21: 638-VEErep-EVD68-VP1-HA2
This sequence is formatted as follows:
Noncoding regions-lower case
NSP ORF UPPER CASE
GOI ORF-UPPERCASE BOLD
auaggcggcgcaugagagaagcccagaccaauuaccuacccaaaAUGGAGAAAGUUCACGUUGACAUCGAGGAAGACA
GCCCAUUCCUCAGAGCUUUGCAGCGGAGCUUCCCGCAGUUUGAGGUAGAAGCCAAGCAGGUCACUGAUAAUGACCAUG
CUAAUGCCAGAGCGUUUUCGCAUCUGGCUUCAAAACUGAUCGAAACGGAGGUGGACCCAUCCGACACGAUCCUUGACA
UUGGAAGUGCGCCCGCCCGCAGAAUGUAUUCUAAGCACAAGUAUCAUUGUAUCUGUCCGAUGAGAUGUGCGGAAGAUC
CGGACAGAUUGUAUAAGUAUGCAACUAAGCUGAAGAAAAACUGUAAGGAAAUAACUGAUAAGGAAUUGGACAAGAAAA
UGAAGGAGCUGGCCGCCGUCAUGAGCGACCCUGACCUGGAAACUGAGACUAUGUGCCUCCACGACGACGAGUCGUGUC
GCUACGAAGGGCAAGUCGCUGUUUACCAGGAUGUAUACGCGGUUGACGGACCGACAAGUCUCUAUCACCAAGCCAAUA
AGGGAGUUAGAGUCGCCUACUGGAUAGGCUUUGACACCACCCCUUUUAUGUUUAAGAACUUGGCUGGAGCAUAUCCAU
CAUACUCUACCAACUGGGCCGACGAAACCGUGUUAACGGCUCGUAACAUAGGCCUAUGCAGCUCUGACGUUAUGGAGC
GGUCACGUAGAGGGAUGUCCAUUCUUAGAAAGAAGUAUUUGAAACCAUCCAACAAUGUUCUAUUCUCUGUUGGCUCGA
CCAUCUACCACGAGAAGAGGGACUUACUGAGGAGCUGGCACCUGCCGUCUGUAUUUCACUUACGUGGCAAGCAAAAUU
ACACAUGUCGGUGUGAGACUAUAGUUAGUUGCGACGGGUACGUCGUUAAAAGAAUAGCUAUCAGUCCAGGCCUGUAUG
GGAAGCCUUCAGGCUAUGCUGCUACGAUGCACCGCGAGGGAUUCUUGUGCUGCAAAGUGACAGACACAUUGAACGGGG
AGAGGGUCUCUUUUCCCGUGUGCACGUAUGUGCCAGCUACAUUGUGUGACCAAAUGACUGGCAUACUGGCAACAGAUG
UCAGUGCGGACGACGCGCAAAAACUGCUGGUUGGGCUCAACCAGCGUAUAGUCGUCAACGGUCGCACCCAGAGAAACA
CCAAUACCAUGAAAAAUUACCUUUUGCCCGUAGUGGCCCAGGCAUUUGCUAGGUGGGCAAAGGAAUAUAAGGAAGAUC
AAGAAGAUGAAAGGCCACUAGGACUACGAGAUAGACAGUUAGUCAUGGGGUGUUGUUGGGCUUUUAGAAGGCACAAGA
UAACAUCUAUUUAUAAGCGCCCGGAUACCCAAACCAUCAUCAAAGUGAACAGCGAUUUCCACUCAUUCGUGCUGCCCA
GGAUAGGCAGUAACACAUUGGAGAUCGGGCUGAGAACAAGAAUCAGGAAAAUGUUAGAGGAGCACAAGGAGCCGUCAC
CUCUCAUUACCGCCGAGGACGUACAAGAAGCUAAGUGCGCAGCCGAUGAGGCUAAGGAGGUGCGUGAAGCCGAGGAGU
UGCGCGCAGCUCUACCACCUUUGGCAGCUGAUGUUGAGGAGCCCACUCUGGAGGCAGACGUCGACUUGAUGUUACAAG
AGGCUGGGGCCGGCUCAGUGGAGACACCUCGUGGCUUGAUAAAGGUUACCAGCUACGAUGGCGAGGACAAGAUCGGCU
CUUACGCUGUGCUUUCUCCGCAGGCUGUACUCAAGAGUGAAAAAUUAUCUUGCAUCCACCCUCUCGCUGAACAAGUCA
UAGUGAUAACACACUCUGGCCGAAAAGGGCGUUAUGCCGUGGAACCAUACCAUGGUAAAGUAGUGGUGCCAGAGGGAC
AUGCAAUACCCGUCCAGGACUUUCAAGCUCUGAGUGAAAGUGCCACCAUUGUGUACAACGAACGUGAGUUCGUAAACA
GGUACCUGCACCAUAUUGCCACACAUGGAGGAGCGCUGAACACUGAUGAAGAAUAUUACAAAACUGUCAAGCCCAGCG
AGCACGACGGCGAAUACCUGUACGACAUCGACAGGAAACAGUGCGUCAAGAAAGAACUAGUCACUGGGCUAGGGCUCA
CAGGCGAGCUGGUGGAUCCUCCCUUCCAUGAAUUCGCCUACGAGAGUCUGAGAACACGACCAGCCGCUCCUUACCAAG
UACCAACCAUAGGGGUGUAUGGCGUGCCAGGAUCAGGCAAGUCUGGCAUCAUUAAAAGCGCAGUCACCAAAAAAGAUC
UAGUGGUGAGCGCCAAGAAAGAAAACUGUGCAGAAAUUAUAAGGGACGUCAAGAAAAUGAAAGGGCUGGACGUCAAUG
CCAGAACUGUGGACUCAGUGCUCUUGAAUGGAUGCAAACACCCCGUAGAGACCCUGUAUAUUGACGAAGCUUUUGCUU
GUCAUGCAGGUACUCUCAGAGCGCUCAUAGCCAUUAUAAGACCUAAAAAGGCAGUGCUCUGCGGGGAUCCCAAACAGU
GCGGUUUUUUUAACAUGAUGUGCCUGAAAGUGCAUUUUAACCACGAGAUUUGCACACAAGUCUUCCACAAAAGCAUCU
CUCGCCGUUGCACUAAAUCUGUGACUUCGGUCGUCUCAACCUUGUUUUACGACAAAAAAAUGAGAACGACGAAUCCGA
AAGAGACUAAGAUUGUGAUUGACACUACCGGCAGUACCAAACCUAAGCAGGACGAUCUCAUUCUCACUUGUUUCAGAG
GGUGGGUGAAGCAGUUGCAAAUAGAUUACAAAGGCAACGAAAUAAUGACGGCAGCUGCCUCUCAAGGGCUGACCCGUA
AAGGUGUGUAUGCCGUUCGGUACAAGGUGAAUGAAAAUCCUCUGUACGCACCCACCUCAGAACAUGUGAACGUCCUAC
UGACCCGCACGGAGGACCGCAUCGUGUGGAAAACACUAGCCGGCGACCCAUGGAUAAAAACACUGACUGCCAAGUACC
CUGGGAAUUUCACUGCCACGAUAGAGGAGUGGCAAGCAGAGCAUGAUGCCAUCAUGAGGCACAUCUUGGAGAGACCGG
ACCCUACCGACGUCUUCCAGAAUAAGGCAAACGUGUGUUGGGCCAAGGCUUUAGUGCCGGUGCUGAAGACCGCUGGCA
UAGACAUGACCACUGAACAAUGGAACACUGUGGAUUAUUUUGAAACGGACAAAGCUCACUCAGCAGAGAUAGUAUUGA
ACCAACUAUGCGUGAGGUUCUUUGGACUCGAUCUGGACUCCGGUCUAUUUUCUGCACCCACUGUUCCGUUAUCCAUUA
GGAAUAAUCACUGGGAUAACUCCCCGUCGCCUAACAUGUACGGGCUGAAUAAAGAAGUGGUCCGUCAGCUCUCUCGCA
GGUACCCACAACUGCCUCGGGCAGUUGCCACUGGAAGAGUCUAUGACAUGAACACUGGUACACUGCGCAAUUAUGAUC
CGCGCAUAAACCUAGUACCUGUAAACAGAAGACUGCCUCAUGCUUUAGUCCUCCACCAUAAUGAACACCCACAGAGUG
ACUUUUCUUCAUUCGUCAGCAAAUUGAAGGGCAGAACUGUCCUGGUGGUCGGGGAAAAGUUGUCCGUCCCAGGCAAAA
UGGUUGACUGGUUGUCAGACCGGCCUGAGGCUACCUUCAGAGCUCGGCUGGAUUUAGGCAUCCCAGGUGAUGUGCCCA
AAUAUGACAUAAUAUUUGUUAAUGUGAGGACCCCAUAUAAAUACCAUCACUAUCAGCAGUGUGAAGACCAUGCCAUUA
AGCUUAGCAUGUUGACCAAGAAAGCUUGUCUGCAUCUGAAUCCCGGCGGAACCUGUGUCAGCAUAGGUUAUGGUUACG
CUGACAGGGCCAGCGAAAGCAUCAUUGGUGCUAUAGCGCGGCAGUUCAAGUUUUCCCGGGUAUGCAAACCGAAAUCCU
CACUUGAAGAGACGGAAGUUCUGUUUGUAUUCAUUGGGUACGAUCGCAAGGCCCGUACGCACAAUCCUUACAAGCUUU
CAUCAACCUUGACCAACAUUUAUACAGGUUCCAGACUCCACGAAGCCGGAUGUGCACCCUCAUAUCAUGUGGUGCGAG
GGGAUAUUGCCACGGCCACCGAAGGAGUGAUUAUAAAUGCUGCUAACAGCAAAGGACAACCUGGCGGAGGGGUGUGCG
GAGCGCUGUAUAAGAAAUUCCCGGAAAGCUUCGAUUUACAGCCGAUCGAAGUAGGAAAAGCGCGACUGGUCAAAGGUG
CAGCUAAACAUAUCAUUCAUGCCGUAGGACCAAACUUCAACAAAGUUUCGGAGGUUGAAGGUGACAAACAGUUGGCAG
AGGCUUAUGAGUCCAUCGCUAAGAUUGUCAACGAUAACAAUUACAAGUCAGUAGCGAUUCCACUGUUGUCCACCGGCA
UCUUUUCCGGGAACAAAGAUCGACUAACCCAAUCAUUGAACCAUUUGCUGACAGCUUUAGACACCACUGAUGCAGAUG
UAGCCAUAUACUGCAGGGACAAGAAAUGGGAAAUGACUCUCAAGGAAGCAGUGGCUAGGAGAGAAGCAGUGGAGGAGA
UAUGCAUAUCCGACGACUCUUCAGUGACAGAACCUGAUGCAGAGCUGGUGAGGGUGCAUCCGAAGAGUUCUUUGGCUG
GAAGGAAGGGCUACAGCACAAGCGAUGGCAAAACUUUCUCAUAUUUGGAAGGGACCAAGUUUCACCAGGCGGCCAAGG
AUAUAGCAGAAAUUAAUGCCAUGUGGCCCGUUGCAACGGAGGCCAAUGAGCAGGUAUGCAUGUAUAUCCUCGGAGAAA
GCAUGAGCAGUAUUAGGUCGAAAUGCCCCGUCGAAGAGUCGGAAGCCUCCACACCACCUAGCACGCUGCCUUGCUUGU
GCAUCCAUGCCAUGACUCCAGAAAGAGUACAGCGCCUAAAAGCCUCACGUCCAGAACAAAUUACUGUGUGCUCAUCCU
UUCCAUUGCCGAAGUAUAGAAUCACUGGUGUGCAGAAGAUCCAAUGCUCCCAGCCUAUAUUGUUCUCACCGAAAGUGC
CUGCGUAUAUUCAUCCAAGGAAGUAUCUCGUGGAAACACCACCGGUAGACGAGACUCCGGAGCCAUCGGCAGAGAACC
AAUCCACAGAGGGGACACCUGAACAACCACCACUUAUAACCGAGGAUGAGACCAGGACUAGAACGCCUGAGCCGAUCA
UCAUCGAAGAGGAAGAAGAGGAUAGCAUAAGUUUGCUGUCAGAUGGCCCGACCCACCAGGUGCUGCAAGUCGAGGCAG
ACAUUCACGGGCCGCCCUCUGUAUCUAGCUCAUCCUGGUCCAUUCCUCAUGCAUCCGACUUUGAUGUGGACAGUUUAU
CCAUACUUGACACCCUGGAGGGAGCUAGCGUGACCAGCGGGGCAACGUCAGCCGAGACUAACUCUUACUUCGCAAAGA
GUAUGGAGUUUCUGGCGCGACCGGUGCCUGCGCCUCGAACAGUAUUCAGGAACCCUCCACAUCCCGCUCCGCGCACAA
GAACACCGUCACUUGCACCCAGCAGGGCCUGCUCGAGAACCAGCCUAGUUUCCACCCCGCCAGGCGUGAAUAGGGUGA
UCACUAGAGAGGAGCUCGAGGCGCUUACCCCGUCACGCACUCCUAGCAGGUCGGUCUCGAGAACCAGCCUGGUCUCCA
ACCCGCCAGGCGUAAAUAGGGUGAUUACAAGAGAGGAGUUUGAGGCGUUCGUAGCACAACAACAAUGACGGUUUGAUG
CGGGUGCAUACAUCUUUUCCUCCGACACCGGUCAAGGGCAUUUACAACAAAAAUCAGUAAGGCAAACGGUGCUAUCCG
AAGUGGUGUUGGAGAGGACCGAAUUGGAGAUUUCGUAUGCCCCGCGCCUCGACCAAGAAAAAGAAGAAUUACUACGCA
AGAAAUUACAGUUAAAUCCCACACCUGCUAACAGAAGCAGAUACCAGUCCAGGAAGGUGGAGAACAUGAAAGCCAUAA
CAGCUAGACGUAUUCUGCAAGGCCUAGGGCAUUAUUUGAAGGCAGAAGGAAAAGUGGAGUGCUACCGAACCCUGCAUC
CUGUUCCUUUGUAUUCAUCUAGUGUGAACCGUGCCUUUUCAAGCCCCAAGGUCGCAGUGGAAGCCUGUAACGCCAUGU
UGAAAGAGAACUUUCCGACUGUGGCUUCUUACUGUAUUAUUCCAGAGUACGAUGCCUAUUUGGACAUGGUUGACGGAG
CUUCAUGCUGCUUAGACACUGCCAGUUUUUGCCCUGCAAAGCUGCGCAGCUUUCCAAAGAAACACUCCUAUUUGGAAC
CCACAAUACGAUCGGCAGUGCCUUCAGCGAUCCAGAACACGCUCCAGAACGUCCUGGCAGCUGCCACAAAAAGAAAUU
GCAAUGUCACGCAAAUGAGAGAAUUGCCCGUAUUGGAUUCGGCGGCCUUUAAUGUGGAAUGCUUCAAGAAAUAUGCGU
GUAAUAAUGAAUAUUGGGAAACGUUUAAAGAAAACCCCAUCAGGCUUACUGAAGAAAACGUGGUAAAUUACAUUACCA
AAUUAAAAGGACCAAAAGCUGCUGCUCUUUUUGCGAAGACACAUAAUUUGAAUAUGUUGCAGGACAUACCAAUGGACA
GGUUUGUAAUGGACUUAAAGAGAGACGUGAAAGUGACUCCAGGAACAAAACAUACUGAAGAACGGCCCAAGGUACAGG
UGAUCCAGGCUGCCGAUCCGCUAGCAACAGCGUAUCUGUGCGGAAUCCACCGAGAGCUGGUUAGGAGAUUAAAUGCGG
UCCUGCUUCCGAACAUUCAUACACUGUUUGAUAUGUCGGCUGAAGACUUUGACGCUAUUAUAGCCGAGCACUUCCAGC
CUGGGGAUUGUGUUCUGGAAACUGACAUCGCGUCGUUUGAUAAAAGUGAGGACGACGCCAUGGCUCUGACCGCGUUAA
UGAUUCUGGAAGACUUAGGUGUGGACGCAGAGCUGUUGACGCUGAUUGAGGCGGCUUUCGGCGAAAUUUCAUCAAUAC
AUUUGCCCACUAAAACUAAAUUUAAAUUCGGAGCCAUGAUGAAAUCUGGAAUGUUCCUCACACUGUUUGUGAACACAG
UCAUUAACAUUGUAAUCGCAAGCAGAGUGUUGAGAGAACGGCUAACCGGAUCACCAUGUGCAGCAUUCAUUGGAGAUG
ACAAUAUCGUGAAAGGAGUCAAAUCGGACAAAUUAAUGGCAGACAGGUGCGCCACCUGGUUGAAUAUGGAAGUCAAGA
UUAUAGAUGCUGUGGUGGGCGAGAAAGCGCCUUAUUUCUGUGGAGGGUUUAUUUUGUGUGACUCCGUGACCGGCACAG
CGUGCCGUGUGGCAGACCCCCUAAAAAGGCUGUUUAAGCUUGGCAAACCUCUGGCAGCAGACGAUGAACAUGAUGAUG
ACAGGAGAAGGGCAUUGCAUGAAGAGUCAACACGCUGGAACCGAGUGGGUAUUCUUUCAGAGCUGUGCAAGGCAGUAG
AAUCAAGGUAUGAAACCGUAGGAACUUCCAUCAUAGUUAUGGCCAUGACUACUCUAGCUAGCAGUGUUAAAUCAUUCA
GCUACCUGAGAGGGGCCCCUAUAACUCUCUACGGCuaaccugaauggacuacgacauagucuaguccgccaagAUGAA
AACUAUAAUCGCUCUGUCAUAUAUCUUUUGUCUGGCAUUGGGCACUUUGGUAGAAAAUUUCCUCUCCAGAGCCGCCUU
GGUAUCAAAAAGAAGUUUUGAGUAUAAAGACCACACGAGCUCUACAGCACGCGCAGACAAGAACUUCUUUAAAUGGAC
GAUAAAUACCAGAAGUUUUGUACAGCUCCGCAGGAAAUUGGAGCUCUUCACAUACCUCCGAUUUGACGCGGAAAUAAC
AAUUUUGACCACAGUUGCGGUUAAUGGUAGUGGAAAUAACACGUACGUAGGCUUGCCUGAUCUGACACUGCAGGCCAU
GUUUGUCCCUACUGGUGCACUCACUCCGGAGAAACAGGACUCCUUCCAUUGGCAGAGCGGGUCAAAUGCGUCAGUGUU
CUUCAAAAUCUCCGAUCCCCCCGCGAGGAUCACUAUUCCCUUUAUGUGUAUAAAUAGCGCCUAUAGCGUUUUUUACGA
UGGCUUUGCCGGCUUUGAAAAGAAUGGGUUGUACGGGAUUAAUCCGGCCGAUACGAUAGGUAACCUGUGUGUACGCAU
AGUUAACGAACACCAGCCAGUGGGUUUCACUGUAACCGUUCGAGUGUACAUGAAACCUAAGCACAUCAAGGCUUGGGC
ACCAAGGCCACCGAGAACCCUCCCAUACAUGGGGUUUCGACAUCAAAAUUCAGAGGGUACUGGACAGGCUGCCGAUCU
CAAGAGUACCCAGGCAGCAAUAGACCAGAUAAACGGCAAACUCAAUCGCGUUAUUGAGAAAACAAACGAAAAGUUCCA
CCAAAUUGAAAAAGAAUUCUCCGAGGUCGAGGGGCGCAUUCAGGAUCUUGAGAAGUACGUUGAAGACACUAAAAUAGA
UCUGUGGAGCUACAACGCGGAGCUCCUGGUCGCUUUGGAGAACCAACAUACCAUAGACCUUACCGAUAGUGAAAUGAA
UAAACUUUUUGAGAAAACGCGACGCCAACUCAGGGAGAAUGCAGAAGAAAUGGGGAACGGUUGUUUUAAAAUAUACCA
UAAGUGCGAUAACGCCUGCAUUGAGUCCAUCCGAAAUGGGACUUAUGACCAUGACGUCUAUCGAGAUGAGGCUCUUAA
CAACCGCUUUCAAAUCAAAGGGGUGGAGCUUAAGUCAGGAUAUAAAGAUUGGAUUCUUUGGAUCUCAUUCGCUAUUUC
UUGUUUUCUUCUUUGUGUCGUCCUUCUGGGGUUCAUUAUGUGGGCUUGCCAGCGGGGAAAUAUACGGUGUAACAUUUG
UAUUugauaaccgcggugucaaaaaccgcguggacgugguuaacaucccugcugggaggaucagccguaauuauuaua
auuggcuuggugcuggcuacuauuguggccauguacgugcugaccaaccagaaacauaauugaauacagcagcaauug
gcaagcugcuuacauagaacucgcggcgauuggcaugccgccuuaaaauuuuuauuuuauuuuuucuuuucuuuuccg
aaucggauuuuguuuuuaauauuucaaaaaaaaaaaaaaaaaaaaaaaaaaaaaaaaaaaaaaaaaaaaa

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition, wherein the composition comprises:
a nanoparticle carrier; and
a nucleic acid, wherein the nucleic acid comprises:
(i) a region encoding for an RNA-dependent RNA polymerase;
(ii) a region encoding for a non-enveloped virus structural protein; and
(iii) a region encoding for a virus protease, wherein the virus structural protein is a substrate for the virus protease.

2. The composition of claim 1, wherein the nucleic acid is an RNA.

3. The composition of claim 1, wherein the virus protease is 3CD.

4. The composition of claim 1, wherein the nucleic acid comprises open reading frames for both (ii) the region encoding the virus structural protein; and (iii) the region encoding the virus protease.

5. The composition of claim 1, wherein the nanoparticle carrier is up to 120 nm in diameter.

6. The composition of claim 1, wherein the nanoparticle carrier is 40 to 80 nm in diameter.

7. The composition of claim 1, wherein the nanoparticle carrier is 50 to 70 nm in diameter.

8. The composition of claim 1, wherein the nanoparticle carrier is dispersed in an aqueous solution.

9. The composition of claim 1, wherein the nanoparticle carrier comprises a cationic lipid.

10. The composition of claim 9, wherein the cationic lipid is 1,2-dioleoyloxy-3 (trimethylammonium)propane (DOTAP), 3β-[N—(N′,N′-dimethylaminoethane) carbamoyl]cholesterol (DC Cholesterol), dimethyldioctadecylammonium (DDA); 1,2-dimyristoyl 3-trimethylammoniumpropane (DMTAP),dipalmitoyl(C16:0)trimethyl ammonium propane (DPTAP), distearoyltrimethylammonium propane (DSTAP), N-[1-(2,3-dioleyloxy)propyl]N,N,Ntrimethylammonium, chloride (DOTMA), N,N-dioleoyl-N,N-dimethylammonium chloride (DODAC), 1,2-dioleoyl-sn-glycero-3-ethylphosphocholine (DOEPC), 1,2-dioleoyl-3-dimethylammonium-propane (DODAP), and 1,2-dilinoleyloxy-3-dimethylaminopropane (DLinDMA),1,1′-((2-(4-(2-((2-(bis(2-hydroxydodecyl)amino)ethyl)(2-hydroxydodecyl)amino)ethyl)piperazin-1-yl)ethyl)azanediyl)bis(dodecan-2-ol) (C12-200), 306Oi10, tetrakis(8-methylnonyl) 3,3′,3″,3″”-(((methylazanediyl) bis(propane-3,1 diyl))bis (azanetriyl))tetrapropionate, 9A1P9, decyl (2-(dioctylammonio)ethyl) phosphate; A2-Iso5-2DC18, ethyl 5,5-di((Z)-heptadec-8-en-1-yl)-1-(3-(pyrrolidin-1-yl)propyl)-2,5-dihydro-1H-imidazole-2-carboxylate; ALC-0315, ((4-hydroxybutyl)azanediyl)bis(hexane-6,1-diyl)bis(2-hexyldecanoate); ALC-0159, 2-[(polyethylene glycol)-2000]-N,N-ditetradecylacetamide; β-sitosterol, (3S,8S,9S,10R,13R,14S,17R)-17-((2R,5R)-5-ethyl-6-methylheptan-2-yl)-10,13-dimethyl-2,3,4,7,8,9,10,11,12,13,14,15,16,17-tetradecahydro-1H-cyclopenta[a]phenanthren-3-ol; BAME-O16B, bis(2-(dodecyldisulfanyl)ethyl) 3,3′-((3-methyl-9-oxo-10-oxa-13,14-dithia-3,6-diazahexacosyl)azanediyl)dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; cKK-E12, 3,6-bis(4-(bis(2-hydroxydodecyl)amino)butyl)piperazine-2,5-dione; DC-Cholesterol, 3β-[N—(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-dioleoyl-sn-glycero-3-phosphoethanolamine; DOSPA, 2,3-dioleyloxy-N-[2-(sperminecarboxamido)ethyl]-N,N-dimethyl-1-propanaminium trifluoroacetate; DSPC, 1,2-distearoyl-sn-glycero-3-phosphocholine; ePC, ethylphosphatidylcholine; FTT5, hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; Lipid H (SM-102), heptadecan-9-yl 8-((2-hydroxyethyl)(6-oxo-6-(undecyloxy)hexyl)amino) octanoate; OF-Deg-Lin, (((3,6-dioxopiperazine-2,5-diyl)bis(butane-4, 1-diyl))bis(azanetriyl))tetrakis(ethane-2,1-diyl) (9Z,9′Z,9″,9″Z,12Z,12′Z,12″Z,12′″Z)-tetrakis (octadeca-9,12-dienoate); PEG2000-DMG, (R)-2,3-bis(myristoyloxy)propyl-1-(methoxy poly(ethylene glycol)2000) carbamate; or TT3, N1,N3,N5-tris(3-(didodecylamino)propyl)benzene-1,3,5-tricarboxamide.

11. The composition of claim 1, wherein the nanoparticle carrier comprises a hydrophobic core.

12. The composition of claim 11, wherein the hydrophobic core comprises an oil.

13. The composition of claim 12, wherein the oil is a-tocopherol, coconut oil, grapeseed oil, lauroyl polyoxylglyceride, mineral oil, monoacylglycerol, palm kernel oil, olive oil, paraffin oil, peanut oil, propolis, squalene, squalane, solanesol, soy lecithin, soybean oil, sunflower oil, a triglyceride, or vitamin E.

14. The composition of claim 13, wherein the triglyceride is capric triglyceride, caprylic triglyceride, a caprylic and capric triglyceride, a triglyceride ester, or myristic acid triglycerin.

15. The composition of claim 1, wherein the nanoparticle carrier comprises an inorganic particle.

16. The composition of claim 11, wherein the hydrophobic core comprises an inorganic particle, and wherein the inorganic particle is within the hydrophobic core.

17. The composition of claim 15, wherein the inorganic particle comprises a metal salt, a metal oxide, a metal hydroxide, or a metal phosphate.

18. The composition of claim 15, wherein the inorganic particle comprises aluminum oxide, aluminum oxyhydroxide, iron oxide, titanium dioxide, or silicon dioxide.

19. The composition of claim 1, wherein the nanoparticle carrier comprises a cationic lipid, an oil, and an inorganic particle.

20. The composition of claim 1, wherein the nanoparticle carrier further comprises a surfactant.

21. The composition of claim 20, wherein the surfactant is a hydrophobic surfactant.

22. The composition of claim 21, wherein the hydrophobic surfactant is sorbitan monolaurate, sorbitan monopalmitate, sorbitan monostearate, sorbitan monooleate, or sorbitan trioleate.

23. The composition of claim 20, wherein the surfactant is a hydrophilic surfactant.

24. The composition of claim 23, wherein the hydrophilic surfactant is a polysorbate.

25. The composition of claim 1, wherein the nanoparticle carrier comprises a cationic lipid, an oil, an inorganic particle, and a surfactant.

26. The composition of claim 11, wherein the hydrophobic core comprises:
one or more inorganic particles;
a phosphate-terminated lipid; and
a surfactant.

27. The composition of claim 26, wherein each inorganic particle is coated with a capping ligand or the surfactant.

28. The composition of claim 26, wherein the phosphate-terminated lipid is trioctylphosphine oxide (TOPO).

29. The composition of claim 26, wherein the surfactant is a phosphorous-terminated surfactant, a carboxylate-terminated surfactant, a sulfate-terminated surfactant, or an amine-terminated surfactant.

30. The composition of claim 26, wherein the surfactant is distearyl phosphatidic acid (DSPA), oleic acid, oleylamine or sodium dodecyl sulfate (SDS).

31. The composition of claim 1, wherein the non-enveloped virus structural protein comprises a protein from a virus from the family Picornaviridae.

32. The composition of claim 1, wherein the non-enveloped virus structural protein comprises a protein from an enterovirus, a coxsackievirus, a rhinovirus, a poliovirus, an echovirus, or a parechovirus.

33. The composition of claim 32, wherein the enterovirus is an enterovirus D68 (EV-D68).

34. The composition of claim 1, wherein the region encoding for the RNA-dependent RNA polymerase comprises a sequence that has at least 85% identity to SEQ ID NO: 8.

35. The composition of claim 1, wherein the region encoding for the non-enveloped virus structural protein comprises a sequence of SEQ ID NO: 18.


SEQUENCE LISTING (25 sequences)

SEQ ID NO: 1 - protein, 175 aa, synthetic construct
MKYLLPTAAAGLLLLAAQPAMAGPGAAAQVQLAESGGGLAQPGGSLRLSCAASGSIFSIDAMGWYRQAIGIQRELVAAITSGGSTNYADSVKGRFTISRGNAKNTVYLQMNSLKPEDTAVYYCNADDETNYERYWGQGTQVTVSSAHHSEDPSARQACTSGAPVPYPDPLEPRAA

SEQ ID NO: 2 - protein, 185 aa, synthetic construct
MKYLLPTAAAGLLLLAAQPAMAGPGAAAQLQLVETGGLVQAGGSLRLSCTASGRTFSSEAMAWFRQAPGKEREFVATINWSSGTDYADSVKGRFTISRDNTKNTVTVYLQMNSLKPEDTAVYYCAADRTGWGASGRDSYEYDLWGQGTQVTVSSEPKTPKPQPARQACTSGAPVPYPDPLEPRAA

SEQ ID NO: 3 - protein, 180 aa, synthetic construct
MKYLLPTAAAGLLLLAAQPAMAGPGAAAQLQLVESGGGLVQPGGSLRLSCAASGRVIGINAMGWYRQAPGKQRELVARVTQAGNINYADSVKDRFTISRDKAENAVYLQMNSLKPEDTAVYYCNGDLFDTPWGPSNDYWGQGTQVTVSSEPKTPKPQPARQACTSGAPVPYPDPLEPRAA

SEQ ID NO: 4 - protein, 176 aa, synthetic construct
MKYLLPTAAAGLLLLAAQPAMAGPGAAAQVQLVESGGGLVQPGGSLRLSCLASGITFTVYRMAWYRQAPGRQRDLVAEVAPGGGTVAANSVKGRFTISRDSAKNTVDLQMNDLKPDDTAVYYCYARNLFTSGEYWGQGTQVTVSSEPKTPKPQPARQACTSGAPVPYPDPLEPRAA

SEQ ID NO: 5 - protein, 29 aa, synthetic construct
CAADRTGWGASGRDSYEDLWGQGTQVTVS

SEQ ID NO: 6 - protein, 25 aa, synthetic construct
CNGDLFDTPWGPSNDYGWGTQVTVS

SEQ ID NO: 7 - protein, 30 aa, synthetic construct
CAADRTGWGASGRDSYEYDLWGQGTQVTVS

SEQ ID NO: 8 - RNA, 7561 nt, Venezuelan equine encephalitis virus
  source 1..7561: mol_type = genomic RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 9 - protein, 51 aa, Venezuelan equine encephalitis virus
RELPVLDSAAFNVECFKKYACNNEYWETFKENPIRLTEENVVNYITKLKGP

SEQ ID NO: 10 - protein, 41 aa, Venezuelan equine encephalitis virus
TQMRELPVLDSAAFNVECFKKYACNNEYWETFKENPIRLTE

SEQ ID NO: 11 - protein, 536 aa, Venezuelan equine encephalitis virus
MKAITARRILQGLGHYLKAEGKVECYRTLHPVPLYSSSVNRAFSSPKVAVEACNAMLKENFPTVASYCIIPEYDAYLDMIDGASCCLDTASFCPAKLRSFPKKHSYLEPTIRSAVPSAIQNTLQNVLAAATKRNCNVTQMRELPVLDSAAFNVECFKKYACNNEYWKTFKENPIRLTEENVINYITKLKGPKAAALYAKTHNLNMLQDIPMDRFVMDLKRDVKVTPGTKHTEERPKVQVIQAADPLATAYLCGIHRELVRRLNAVLLPNIHTLFDMSAEDFDAIIAEHFQPGDCVLETDIASFDKSEDDAMALTAMMILEDLGVDAELLTLIEAAFGEISSIHLPTKTKFKFGAMMKSGMFLTLFVNTVINIVIASRVLRERLTGSPCAAFIGDDNIVKGVKSDKLMADRCATWLNMEVKIIDAVVGEKAPYFCGGFILCDSVTGTACRVADPLKRLFKLGKPLAADDEHDDDRRRALHEESTRWNRVGILPELCKAVESRYETVGTSVIVMAMATLASSVKSFSYLRGAPITLYG

SEQ ID NO: 12 - RNA, 8983 nt, synthetic construct
  source 1..8983: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatggagttcggtcttagctgggtgtttcttgtcgccctgttcagaggggtacaatgcggcccgggagcggccgctcagttgcagctggtggagtcaggtggaggcttggtgcagcctggggggtctctgagactctcctgtgcagcctctggccgcgtcatcggaatcaatgccatgggctggtaccgccaggctccagggaagcagcgcgagttggtcgcacgagttactcaagctggtaacatcaactatgcagactccgtgaaggaccgattcaccatctccagagacaaggccgagaacgcggtgtatctacaaatgaacagcctcaaacctgaggacacggccgtctactactgtaatggagatcttttcgatacgccttggggtccatcaaatgactactggggccaggggacccaagtcaccgtctcctcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 13 - RNA, 8971 nt, synthetic construct
  source 1..8971: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccgccaccatggagttcggtcttagctgggtgtttcttgtcgccctgttcagaggggtacaatgcggcccgggagcggccgctcaggtgcagctggtggagtcggggggaggtttggtgcagcctggagggtctctgagactctcctgtttagcctctggaatcaccttcactgtctatcgcatggcctggtaccgtcaggctccggggaggcagcgcgacttggtcgcagaagtagctcctggtggtggaacggtggctgcaaactccgtgaagggccgattcaccatctccagagacagcgccaagaacacggtggatctgcaaatgaacgacctgaaacctgacgatacggccgtctattattgttatgcacgtaatcttttcacgtcgggggagtattggggccaggggacccaggtcaccgtctcctcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 14 - RNA, 381 nt, synthetic construct
  source 1..381: mol_type = other RNA
ggcccgggagcggccgctcagttgcagctggtggagtcaggtggaggcttggtgcagcctggggggtctctgagactctcctgtgcagcctctggccgcgtcatcggaatcaatgccatgggctggtaccgccaggctccagggaagcagcgcgagttggtcgcacgagttactcaagctggtaacatcaactatgcagactccgtgaaggaccgattcaccatctccagagacaaggccgagaacgcggtgtatctacaaatgaacagcctcaaacctgaggacacggccgtctactactgtaatggagatcttttcgatacgccttggggtccatcaaatgactactggggccaggggacccaagtcaccgtctcctca

SEQ ID NO: 15 - RNA, 369 nt, synthetic construct
  source 1..369: mol_type = other RNA
ggcccgggagcggccgctcaggtgcagctggtggagtcggggggaggtttggtgcagcctggagggtctctgagactctcctgtttagcctctggaatcaccttcactgtctatcgcatggcctggtaccgtcaggctccggggaggcagcgcgacttggtcgcagaagtagctcctggtggtggaacggtggctgcaaactccgtgaagggccgattcaccatctccagagacagcgccaagaacacggtggatctgcaaatgaacgacctgaaacctgacgatacggccgtctattattgttatgcacgtaatcttttcacgtcgggggagtattggggccaggggacccaggtcaccgtctcctca

SEQ ID NO: 16 - RNA, 681 nt, Homo sapiens
  source 1..681: mol_type = genomic RNA
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 17 - RNA, 54 nt, unidentified
  source 1..54: mol_type = unassigned RNA
gagttcggtcttagctgggtgtttcttgtcgccctgttcagaggggtacaatgc

SEQ ID NO: 18 - RNA, 12956 nt, synthetic construct
  source 1..12956: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 19 - RNA, 10444 nt, synthetic construct
  source 1..10444: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacctgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 20 - RNA, 12439 nt, synthetic construct
  source 1..12439: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgggtgctcaggtaaccaggcagcaaaccggtactcatgaaaatgccaacatagctactaatggctcccatattacgtacaatcaaatcaatttctacaaggatagttacgctgcgtccgcttctaagcaggacttcagtcaggatcctagcaagtttacggaacccgtagttgaaggccttaaggcaggggcacctgtccttaagtcaccgagtgcggaggcttgcggttactctgaccgagtactgcagcttaagctcgggaactctgccatagttacgcaggaagcggcaaactattgctgcgcgtacggggagtggccgaactacctgccagaccatgaggcggtcgctatagacaagccaacacaacctgagacagccacggatcggttctatactcttaaaagcgtaaaatgggagactggctccacaggatggtggtggaagctcccagatgcccttaacaatatcgggatgtttggccagaatgttcaacaccattacctgtatcgcagtggcttcctcattcacgtccagtgtaatgccacaaagtttcatcagggggctctccttgtggtggcgatcccagagcatcagaggggtgcacataatactaatactagtcctggtttcgatgatataatgaaaggggaggaaggagggacgtttaatcatccttatgtcctggatgacgggacctcattggcgtgtgcgacgatcttccctcaccagtggattaatctccggaccaataacagtgcgactatcgtacttccatggatgaacgcggctccgatggattttcccctgaggcataatcagtggacattggctattattccggtcgtacccctgggtactagaaccactagctcaatggttcccataactgtatctattgcgccaatgtgctgtgaatttaatgggctccggcacgctatcacacaaggcgttcctacgtatctcttgccaggctcaggtcagttcctcactactgatgaccatagctccgcacctgccctcccctgttttaacccaacacccgagatgcatatcccagggcaagtccgaaacatgcttgaggttgttcaggtagaatctatgatggagatcaataacacagagagtgcggtagggatggagcgccttaaggttgacatctccgcattgaccgatgttgaccaacttttgtttaacattcccctggatatacagctcgatggccccttgcggaacacgttggtcggaaatatctccaggtactacactcattggtccggcagtctcgaaatgacatttatgttttgcggcagtttcatggcagcgggcaaactgatcctgtgttatacacccccaggcggtagttgtccaacgacgcgagagacggcgatgctcggcacacatatagtgtgggattttggcttgcaatcctcagttaccctcatcataccgtggataagcggcagccattatagaatgttcaacaatgacgctaaaagtacgaacgccaatgtgggatatgtgacctgctttatgcagacgaatctcatcgtaccttctgagtcctcagacacatgcagtttgataggtttcatagccgcaaaggacgatttcagtcttagacttatgcgggacagtccggacattggtcaactggatcaccttcatgctgcggaggcagcatatcagatcgaatcaataattaaaactgctaccgacacagtcaagtccgagataaacgctgaactgggcgtcgtcccgagtcttaatgcagtggaaaccggagccacttctaatactgagccagaagaagcaattcaaactcgaactgtgatcaaccaacacggtgtaagcgagactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatgagcattgctaatgcaaattataagggaaaagagagagcaccgaacgcgttgtctgcaattatcggcaatcgggactcagtcaagactatgccacataatatagtcaatacccgccggaagcggggtagcggagaggggcgcgggtcactgttgacgtgcggggacgtggaagaaaatccggggcctggtccaggctttgactttgctcaggcgataatgaagaaaaacacggttatcgcacgaactgaaaagggcgaatttaccatgctcggcgtgtacgatcgagtcgcggttatcccgacacacgcttccgtgggggaaaccatatatatcaacgatgtagaaaccaaagtcctcgacgcgtgtgcactgagggatcttacagacactaacctggagatcacaatagtgaagctggatcgaaatcagaagttccgagacatccgccattttttgcctagatatgaagacgactacaatgatgctgtactgtccgtgcatacttcaaagtttcctaacatgtacatccctgttggccaggtcactaattacggttttcttaacctgggtgggacgcctactcataggatactgatgtacaattttcctactagagctggacaatgtggtggcgtagtgactaccaccgggaaagtcattggcatacacgtaggaggtaacggggcgcagggattcgcggccatgcttctgcacagctatttctccgacacacaaggtgaaatagtttcatcagagaaatccggtgtgtgcattaacgctcccgcgaaaactaaacttcagcccagcgtgtttcatcaagtattcgaaggaagcaaggaaccggctgtactgaaccccaaggacccccggcttaaaacggatttcgaagaagcgatattttcaaaatatactggtaacaaaatcatgctgatggatgagtatatggaagaagctgtggaccactatgtagggtgcctggaaccgctcgacatctctgtggacccgatcccactcgagtccgctatgtacggcatggacggcctcgaggctttggaccttacaactagcgcgggctttccgtatcttttgcaaggtaagaaaaagcgcgacatctttaaccgccacaccagggatacgagcgagatgacaaaaatgcttgaaaaatatggggtcgatcttccttttgtcactttcgtgaaggacgaattgagatcccgagaaaaggtcgagaaaggtaagtctcgcctcattgaagccagttcacttaatgatagtgttgcgatgcgagttgcttttggtaacctttacgcaacatttcataacaatccaggcacggctacaggatcagcagtaggttgcgacccagacatcttttggtcaaaaatccccattctgctggacggtgaaatttttgcctttgactataccggatacgacgcatccttgtcccctgtatggttcgcatgtctcaaaaaagtcctgataaaactcggttacactcaccagactagttttatagactatctgtgtcatagtgttcacctctacaaagataaaaaatatattgtgaacggtggtatgccgtctggtagttccggaacttccatatttaacacaatgattaataatatcattataaggacgcttctcatcagggtctacaagggtatcgatctggatcaattcaagatgatagcatacggcgacgacgtcattgcttcttacccccataagattgatccaggtctgctggcggaagccggcaagcaatatggactggttatgacacccgctgacaaaggaaccagtttcatcgacacgaattgggaaaacgtgacgttcctgaagcgatacttcagagcagacgatcaatatccctttcttatccatcccgttatgccaatgaaggagatacacgagtcaatccgatggacaaaagacccacggaacacacaagatcacgtccgatcactctgttatcttgcctggcacaatggggaggaggcgtataatgagttctgccggaagattcgaagcgtaccagtaggccgagcactgactctccctgcttattcaagtctgcggcggaagtggttggattccttctagtaaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 21 - RNA, 9118 nt, synthetic construct
  source 1..9118: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctggccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatgaaaactataatcgctctgtcatatatcttttgtctggcattgggcactttggtagaaaatttcctctccagagccgccttggtatcaaaaagaagttttgagtataaagaccacacgagctctacagcacgcgcagacaagaacttctttaaatggacgataaataccagaagttttgtacagctccgcaggaaattggagctcttcacatacctccgatttgacgcggaaataacaattttgaccacagttgcggttaatggtagtggaaataacacgtacgtaggcttgcctgatctgacactgcaggccatgtttgtccctactggtgcactcactccggagaaacaggactccttccattggcagagcgggtcaaatgcgtcagtgttcttcaaaatctccgatccccccgcgaggatcactattccctttatgtgtataaatagcgcctatagcgttttttacgatggctttgccggctttgaaaagaatgggttgtacgggattaatccggccgatacgataggtaacctgtgtgtacgcatagttaacgaacaccagccagtgggtttcactgtaaccgttcgagtgtacatgaaacctaagcacatcaaggcttgggcaccaaggccaccgagaaccctcccatacatggggtttcgacatcaaaattcagagggtactggacaggctgccgatctcaagagtacccaggcagcaatagaccagataaacggcaaactcaatcgcgttattgagaaaacaaacgaaaagttccaccaaattgaaaaagaattctccgaggtcgaggggcgcattcaggatcttgagaagtacgttgaagacactaaaatagatctgtggagctacaacgcggagctcctggtcgctttggagaaccaacataccatagaccttaccgatagtgaaatgaataaactttttgagaaaacgcgacgccaactcagggagaatgcagaagaaatggggaacggttgttttaaaatataccataagtgcgataacgcctgcattgagtccatccgaaatgggacttatgaccatgacgtctatcgagatgaggctcttaacaaccgctttcaaatcaaaggggtggagcttaagtcaggatataaagattggattctttggatctcattcgctatttcttgttttcttctttgtgtcgtccttctggggttcattatgtgggcttgccagcggggaaatatacggtgtaacatttgtatttgataaccgcggtgtcaaaaaccgcgtggacgtggttaacatccctgctgggaggatcagccgtaattattataattggcttggtgctggctactattgtggccatgtacgtgctgaccaaccagaaacataattgaatacagcagcaattggcaagctgcttacatagaactcgcggcgattggcatgccgccttaaaatttttattttattttttcttttcttttccgaatcggattttgtttttaatatttcaaaaaaaaaaaaaaaaaaaaaaaaaaaaaaaaaaaaaaaaaaaaa

SEQ ID NO: 22 - protein, 26 aa, synthetic construct
CNGDLFDTPWGPSNDYWGQGTQVTVS

SEQ ID NO: 23 - protein, 22 aa, synthetic construct
CNADDETNYERYWGQGTQVTVS

SEQ ID NO: 24 - protein, 22 aa, synthetic construct
CYARNLFTSGEYWGQGTQVTVS

SEQ ID NO: 25 - protein, 31 aa, synthetic construct
CARQSPLSGWDCPRTAYDYDYWGQGTEVTVS